(19) United States
(12) Patent Application Publication
(10) Pub. No.: US 2025/0093354 A1
(43) Pub. Date: Mar. 20, 2025

(54) METHOD FOR HIGH THROUGHPUT PEPTIDE-MHC AFFINITY SCREENING FOR TCR LIGANDS

(71) Applicant: Immatics Biotechnologies GmbH, Tuebingen (DE)

(72) Inventors: Andreas MORITZ, Tübingen (DE); Dominik MAURER, Tübingen (DE); Sebastian BUNK, Tübingen (DE); Claudia WAGNER, Tübingen (DE)

(21) Appl. No.: 18/919,930

(22) Filed: Oct. 18, 2024

Related U.S. Application Data
(62) Division of application No. 16/569,691, filed on Sep. 13, 2019, now Pat. No. 12,188,932.
(60) Provisional application No. 62/731,337, filed on Sep. 14, 2018.
(60) Provisional application No. 62/873,102, filed on Jul. 11, 2019.

(30) Foreign Application Priority Data
Sep. 14, 2018 (DE) 102018122546.6

(51) Int. Cl. G01N 33/569; A61K 38/17; A61K 39/00; A61K 39/395; C07K 14/74

(52) U.S. Cl. CPC G01N 33/56977; A61K 38/1774; A61K 39/00; A61K 39/39541; C07K 14/70539; A61K 2039/505; A61K 2039/57; G01N 2333/7051; G01N 2333/70539

(57) ABSTRACT

The present invention relates to a method for high throughput screening for a TCR-binding peptide ligand/MHC molecule complex, comprising a stabilized peptide-MHC molecule and respective uses of said method. The present invention further relates to polypeptides comprising or consisting of stabilized MHC molecules or peptide binding fragments thereof, pharmaceutical compositions comprising said polypeptides, vaccines comprising said pharmaceutical composition and uses of said vaccine for the manufacturing of a medicament and/or in the prevention of cancer The present invention further relates to nucleic acids encoding said polypeptides and vectors comprising said nucleic acids.

### CROSS-REFERENCE TO RELATED APPLICATIONS

This application is a Divisional Application of U.S. application Ser. No. 16/569,691, filed 13 Sep. 2019, which claims priority to U.S. Provisional Application No. 62/731,337, filed 14 Sep. 2018, German Patent Application 102018122546.6, filed 14 Sep. 2018, and United States Provisional Application No. 62/873, 102, filed 11 Jul. 2019, the entire contents of each of which are incorporated herein by reference.

### REFERENCE TO SEQUENCE LISTING SUBMITTED AS AN XML FILE (.xml)

A Sequence Listing is submitted herewith as an XML file named “3000058-013003_Sequence_Listing” created on 6 Dec. 2024 and having a size of 360,524 bytes as permitted under 37 C.F.R. § 1.821 (c). The material in the aforementioned file is hereby incorporated by reference in its entirety.

The present invention relates to a method for high throughput screening for a TCR-binding peptide ligand/MHC molecule complex, comprising a stabilized peptide-MHC molecule and respective uses of said method. The present invention further relates to polypeptides comprising or consisting of stabilized MHC molecules or peptide binding fragments thereof, pharmaceutical compositions comprising said polypeptides, vaccines comprising said pharmaceutical composition and uses of said vaccine for the manufacturing of a medicament and/or in the prevention of cancer The present invention further relates to nucleic acids encoding said polypeptides and vectors comprising said nucleic acids.

### BACKGROUND OF THE INVENTION

### Field

Presentation of peptides on cell surface MHC molecules plays a fundamental role for the immune response against viral infection or cancer (1). MHC class I molecules are trimeric complexes that consist of a polymorphic heavy chain, beta-2 microglobulin (β₂m) and a peptide ligand, typically between 8 and 10 amino acids long and derived from degradation of cytosolic proteins. T cells can recognize specific peptide-MHC complexes (pMHC) with their clone-specific T cell receptor (TCR) and initiate an immune response.

Production of soluble pMHC complexes is important for many different applications in scientific and clinical fields that are centered around the interaction between pMHCs and TCRs. They were first generated using protein expression and refolding techniques in 1992 and have since then been used for many applications, e.g. identification of antigen-specific T cells through flow cytometry or affinity measurements of the TCR-pMHC interaction (2 to 5).

The affinity of the TCR towards its cognate pMHC has a substantial impact on the functionality of the expressing T cell (6). Thus, efforts have been made to improve the affinity of low-affinity TCRs to reach optimal levels for clinical applications (7). Extensive maturation experiments have produced TCRs with picomolar affinities, a range normally reserved to antibodies. They bind targeted pMHCs with long interaction half-lives even in monomeric form and have thus attracted attention as tumor cell engaging component in bi-specific T cell engager formats (8, 9).

WO 2013/030620 discloses recombinant MHC class I molecules which are produced in bacteria and are present as an insoluble attachment body for a detection of epitope-specific CTL. These are first denatured in a solution of a chaotropic agent. The chaotrope is then removed in the presence of the desired peptide (renaturing, refolding) and the peptide class I complex is separated by gel filtration chromatography from the unfolded protein. WO 2013/030620 presents a gene for encoding an MHC class I molecule, the MHC class I molecule having an alpha 1 helix and an alpha 2 helix and the gene being encoded such that a bond is formed between the alpha 1 helix and the alpha 2 helix in the MHC class I molecule. Thus, a kit for analysis of T cell frequencies can be provided. Amino acid 139 is substituted by a cysteine so as to provide Cys-139, the amino acid 84 is substituted by the cysteine so as to provide Cys-84 or the amino acid 85 is substituted by the cysteine so as to provide Cys-85, the disulfide bridge is formed between the alpha-1 helix and the alpha-2 helix in the MHC class I heavy chain between Cys-139 and Cys-84 or between Cys-139 and Cys-85.

US 2009-0117153 discloses a so-called disulfide trap, comprising an antigen peptide covalently attached to an MHC class I heavy chain molecule by a disulfide bond extending between two cysteines. In some configurations, a disulfide trap, such as a disulfide trap single chain trimer (dtSCT), can comprise a single contiguous polypeptide chain. Upon synthesis in a cell, a disulfide trap oxidizes properly in the ER, and can be recognized by T cells. In some configurations, a peptide moiety of a disulfide trap is not displaced by high-affinity competitor peptides, even if the peptide binds the heavy chain relatively weakly. In various configurations, a disulfide trap can be used for vaccination, to elicit CD8 T cells, and in multivalent MHC/peptide reagents for the enumeration and tracking of T cells. Also disclosed are nucleic acids comprising a sequence encoding a disulfide trap. Such nucleic acids, which can be DNA vectors, can be used as vaccines.

Zeynep Hein, et al. (in: Peptide-independent stabilization of MHC class I molecules breaches cellular quality control. J Cell Sci 2014 127: 2885-2897) describe a variant of the murine MHC-I allotype H-2Kb, in which the α1 and α2 helices are connected by a disulfide bond close to the F-pocket, restricting their mobility. The C84-C139 disulfide bond allows normal PLC interaction and antigen presentation but renders MHC-I surface expression TAP-and tapasin-independent, accelerates anterograde transport, and greatly decreases the rate of MHC-I endocytosis.

WO 2011/101681 discloses disulfide bond stabilized recombinant MHC class II molecules that are linked by a disulfide bond between cysteine residues located in the α2 domain of said a chain and the β2 domain of said β chain, wherein said cysteine residues are not present in native MHC class II α2 and β2 domains.

WO 2018/029350 discloses a Kₒₙ-rate assay and an improved TCR ligand K_{off}-rate assay, which enables a broader application through a novel combination with UV peptide exchange technology. The disclosure enables K_{off}-rate MHC monomer preparation in a high throughput manner, which can then be used to screen TCR candidates for extended peptide libraries in assays such as the TCR ligand K_{off}-rate assay that was previously not feasible. Further, the UV peptide exchange with the K_{off}-rate MHC monomers allows the analysis of TCR candidates recognizing specific peptides carrying the amino acid cysteine, which previously could interfere with or even abolish the K_{off}-rate measurement.

Newell et al. (in: Newell EW, “Higher Throughput Methods of Identifying T Cell Epitopes for Studying Outcomes of Altered Antigen Processing and Presentation.” Frontiers in Immunology. 2013; 4:430) disclose high content combinatorial peptide-MHC tetramer staining using mass cytometry.

Bakker et al. (in: Bakker AH, Hoppes R, Linnemann C, et al., “Conditional MHC class I ligands and peptide exchange technology for the human MHC gene products HLA-A1, -A3, -A11, and -B7.” PNAS 2008; 105(10):3825-3830) disclose conditional ligands that disintegrate upon exposure to long-wavelength UV light that can be designed for the human MHC molecule HLA-A2. This peptide-exchange technology allegedly can be developed into a generally applicable approach for high throughput MHC based applications for an analysis of cytotoxic T cell immunity.

Cochran and Stern (in: “A diverse set of oligomeric class II MHC-peptide complexes for probing T-cell receptor interactions.” Chem Biol. 2000 September;7(9):683-96) disclose tools to study the molecular mechanisms responsible for initiation of activation processes in T-cells. A topologically diverse set of oligomers of the human MHC protein HLA-DR1, varying in size from dimers to tetramers, was produced by varying the location of an introduced cysteine residue and the number and spacing of sulfhydryl-reactive groups carried on novel and commercially available cross-linking reagents. Fluorescent probes incorporated into the cross-linking reagents facilitated measurement of oligomer binding to the T-cell surface. Oligomeric MHC-peptide complexes, including a variety of MHC dimers, trimers and tetramers, bound to T-cells and initiated T-cell activation processes in an antigen-specific manner.

Chong et al. (in: “High-throughput and Sensitive Immunopeptidomics Platform Reveals Profound Interferon-γ-Mediated Remodeling of the Human Leukocyte Antigen (HLA) Ligandome.” Molecular & Cellular Proteomics: MCP. 2018;17(3):533-548) disclose a high-throughput, reproducible and sensitive method for sequential immuno-affinity purification of HLA-I and -II peptides from up to 96 samples in a plate format, suitable for both cell lines and tissues. The method is directed at improving the allegedly most critical step in the immunopeptidomics pipeline, the sample preparation, as it determines the overall peptide yield and reproducibility.

Luimstra et al. (in: Luimstra JJ, Garstka MA, Roex MCJ, et al. “A flexible MHC class I multimer loading system for large-scale detection of antigen-specific T cells.” J Exp Med 2018; 215(5):1493-1504) disclose an allegedly simple, fast, flexible, and efficient method to generate many different MHC class I (MHC I) multimers in parallel using temperature-mediated peptide exchange. They designed conditional peptides for HLA-A*02:01 and H-2K^{b} that form stable peptide-MHC I complexes at low temperatures, but dissociate when exposed to a defined elevated temperature. The resulting conditional MHC I complexes, either alone or prepared as ready-to-use multimers, can swiftly be loaded with peptides of choice without additional handling and within a short time frame.

### SUMMARY

A potential downside of TCR affinity enhancement is the introduction of off-target toxicities. Due to the inherent cross-reactivity of TCRs these can arise by unknowingly increasing the affinity towards other pMHCs as well (10). Multiple cases like these have already been reported in clinical studies (11 to 13).

Comprehensive screening is therefore necessary not only to ensure efficacy but also specificity and safety of therapeutic candidates (14). This is a task of high complexity given the currently established size of the immunopeptidome, with at least 150,000 MHC class I ligand peptides identified by mass spectrometry, and the available methods for pMHC generation (15).

The large-scale generation of pMHC libraries and subsequent high throughput binding screenings of TCRs, e.g. for binding motif generation or the direct identification and characterization of potentially cross-reactive peptides are still difficult to achieve using common technologies in the art, like the ones above. This difficulty extends to the preparation of high quality pMHC complexes even in lower numbers for individuals or institutions without the necessary technically challenging facilities to produce pMHC, e.g. for time sensitive on demand production in clinical settings. It is therefore an object of the present invention, to provide improved strategies in this field. Other objects and aspects of the present invention will become apparent to the person of skill upon reading the following description of the invention.

According to a first aspect thereof, the above object of the invention is solved by a method for screening for a TCR-binding peptide ligand/MHC molecule complex (pMHC), comprising the steps of:
a) providing a suitably stabilized MHC molecule, wherein said MHC molecule comprises at least one artificially introduced covalent bridge:
   (i) between amino acids of the alpha1 domain and amino acids of the alpha2 domain of said stabilized MHC molecule in case of MHC I; and/or
   (ii) between two amino acids of the alpha1 domain of said stabilized MHC molecule in case of MHC I; or
   (iii) between two amino acids of the alpha1 domain or two amino acids of the beta1 domain of said stabilized MHC molecule in case of MHC II; and/or
   (iv) between one amino acid of the alpha1 domain and one amino acid of the beta1 domain of said stabilized MHC molecule in case of MHC II;
b) contacting said suitably stabilized MHC molecule with a multitude of peptide ligands thereof, to form peptide ligand/MHC (pMHC) molecule complexes, and
c) screening said pMHC molecule complexes for TCR-binding.

Preferred is a method according to the invention, wherein said stabilized MHC molecule encompasses at least one artificially introduced covalent disulfide bridge between two amino acids, more preferable at least one artificially introduced covalent bridge between amino acids between α-helices, for example by (i) mutating the amino acid at position 84, a tyrosine in the majority of HLAs (see FIG. 13) and an amino acid at position 139, a alanine in the majority of HLAS (see FIG. 13) into cysteines and/or (ii) mutating an amino acid at position 22, a phenylalanine in the majority of HLAs (see FIG. 13) and an amino acid at position 71, a serine in the majority of HLAs (see FIG. 13) and/or (iii) mutating an amino acid at position 51, a tryptophan in the majority of HLAs (see FIG. 13), and an amino acid at position 175, a glycine in the majority of HLAs (see FIG. 13), or (iv) mutating an amino acid at position 22, a phenylalanine in the majority of HLAs (see FIG. 13) and an amino acid at position 71, a serine in the majority of HLAs (see FIG. 13) and mutating an amino acid at position 51, a tryptophan in the majority of HLAs (see FIG. 13), and an amino acid at position 175, a glycine in the majority of HLAs (see FIG. 13) of MHC I (based on IGMT numbering excluding the first 24 amino acids). Such a stabilized MHC molecule may be referred to as disulfide-modified MHC molecule or disulfide-modified MHC mutant. Either the TCR or the MHC molecule can be suitably immobilized on a solid surface, such as a chip, glass slide, biosensor or bead, in particular as a high-throughput screening format.

In a second aspect the present invention provides a polypeptide comprising or consisting of a stabilized MHC molecule or a peptide binding fragment thereof, which comprises at least one artificially introduced covalent bridge:
(i) between two amino acids in the alpha1 domain of an MHC I; and/or
(ii) between one amino acid in the alpha1 domain of an MHC I and one amino acid in the alpha2 domain of an MCH I within amino acid positions 160 to 179; or
(iii) two amino acids in the alpha1 domain or two amino acids in the beta1 domain of an MHC II; and/or
(iv) between one amino acid in the alpha1 domain of a MHC II and one amino acid in the beta1 domain of a MHC II.

Two amino acid positions that are modified, e.g. by artificially introducing a cysteine residue instead of the naturally occurring amino acid, to form a covalent bridge are selected based on their relative distance. If two amino acids in an MHC I or MHC II that are not linked to each other by peptide bonds naturally have a distance to each other that is similar to the distance of a covalent bond, it is preferred that they are substituted by an amino acid that can form a covalent bond, e.g. a cysteine. Thus, preferably two amino acids are modified that have a distance of between 3 to 7.5 Å in the folded protein (determined between the alpha carbons of the respective amino acids). The 3D structures of a large number of MHC I and MHC II molecules are known and the skilled person can use standard software to determine the distance between two given amino acids within the folded molecules.

If the two amino acids are modified in the alpha1 domain of MHC I it is preferred that one amino acid is modified in the β1 unit and one in the α1 unit of MHC I. Particularly, suitable regions within the β1 unit are within amino acid positions 12 to 32, preferably within amino acid positions 17 to 27, more preferably within amino acid positions 20 to 24 and most preferably amino acid position 22. Particularly, suitable regions within the α1 unit are within amino acid positions 61 to 81, preferably within amino acid positions 66 to 76, more preferably within amino acid positions 69 to 73 and most preferably amino acid position 71. In each case, the two amino acids are preferably selected within the respectively indicated amino acid stretches to have a distance of between 3 to 7.5 Å in the folded MHC I or MHC II protein (determined between the alpha carbons of the respective amino acids).

If the two amino acids are modified in the alpha1 domain of MHC II it is preferred that one amino acid is modified in the β1 unit and one in the α1 unit of MHC II. Particularly, suitable regions within the β1 unit are within amino acid positions 10 to 40, preferably within amino acid positions 13 to 35, more preferably within amino acid positions 22 to 25 and most preferably amino acid position 22. Particularly, suitable regions within the α1 unit are within amino acid positions 45 to 78, preferably within amino acid positions 50 to 70, more preferably within amino acid positions 56 to 66 and most preferably amino acid position 59. In each case, the two amino acids are preferably selected within the respectively indicated amino acid stretches to have a distance of between 3 to 7.5 Å in the folded MHC I or MHC II protein (determined between the alpha carbons of the respective amino acids).

If the two amino acids are modified in the beta1 domain of MHC II it is preferred that one amino acid is modified in the β3 unit and one in the α3 unit of MHC II. Particularly, suitable regions within the β3 unit are within amino acid positions 15 to 53, preferably within amino acid positions 17 to 41, more preferably within amino acid positions 21 to 28 and most preferably amino acid position 26. Particularly, suitable regions within the α3 unit are within amino acid positions 52 to 88, preferably within amino acid positions 66 to 76, more preferably within amino acid positions 65 to 80 and most preferably amino acid position 75. In each case, the two amino acids are preferably selected within the respectively indicated amino acid stretches to have a distance of between 3 to 7.5 Å in the folded MHC I or MHC II protein (determined between the alpha carbons of the respective amino acids).

If one amino acid is modified in the alpha1 domain of an MHC I and one amino acid in the alpha2 domain of an MCH I within amino acid positions 160 to 179, it is preferred that the one amino acid in the alpha1 domain is modified in the α1 unit, preferably within amino acid positions 50 to 70, more preferably within amino acid positions 50 to 60, more preferably 50 to 54 and most preferably amino acid position 51. It is preferred that the other amino acid in the alpha2 domain is modified in the α2 unit, suitable regions are within amino acid positions 165 to 178, preferably within amino acid positions 170 to 177, more preferably within amino acid positions 173 to 176 and most preferably amino acid position 175. In each case the two amino acids are preferably selected within the respectively indicated amino acid stretches to have a distance of between 3 to 7.5 Å in the folded MHC I protein. Thus, in a particularly preferred embodiment the stabilized MHC I comprises a modified amino acid at position 51 and at position 175.

If one amino acid is modified in the alpha1 domain of a MHC II and one amino acid in the beta1 domain of a MHC II it is in one embodiment preferred that one amino acid in the alpha1 domain is modified in the α1 unit. In one pair of modified amino acids the first modified amino acid is within amino acid positions 50 to 70, more preferably within amino acid positions 50 to 60, more preferably 50 to 54 and most preferably amino acid position 51. The other modified amino acid within the beta1 domain is preferably within the α3 unit spanning amino acid positions 70 to 95, preferably within amino acid positions 74 to 94, preferably within amino acid positions 83 to 93, more preferably within amino acid positions 87 to 92 and most preferably amino acid position 89. In another pair the first modified amino acid is within amino acid positions 70 to 90, more preferably within amino acid positions 70 to 85, more preferably 72 to 79 and most preferably amino acid position 76. The other modified amino acid within the beta1 domain is preferably within the a3 unit spanning amino acid positions 50 to 95, preferably within amino acid positions 50 to 65, preferably within amino acid positions 50 to 60, more preferably within amino acid positions 50 to 55 and most preferably amino acid position 53. In each case, the two amino acids are preferably selected within the respectively indicated amino acid stretches to have a distance of between 3 to 7.5 Å in the folded MHC II protein.

It is further preferred that within one MHC comprises two pairs of modified amino acids. Particularly, preferred combinations that may be combined are indicated under (i) and (ii) above for MHC I and under (iii) and (iv) above for MHC II. Thus, it is preferred that the first pair of modified amino acids comprises one amino acid that is modified in the β1 unit and one in the α1 unit of MHC I. Particularly, suitable regions within the β1 unit are within amino acid positions 12 to 32, preferably within amino acid positions 17 to 27, more preferably within amino acid positions 20 to 24 and most preferably amino acid position 22. Particularly, suitable regions within the α1 unit are within amino acid positions 61 to 81, preferably within amino acid positions 66 to 76, more preferably within amino acid positions 69 to 73 and most preferably amino acid position 71. The second pair of modified amino acids comprise one amino acid that is modified in the alpha1 domain of an MHC I and one amino acid in the alpha2 domain of an MCH I within amino acid positions 160 to 179. It is preferred that the one amino acid in the alpha1 domain is modified in the α1 unit, preferably within amino acid positions 50 to 70, more preferably within amino acid positions 50 to 60, more preferably 50 to 54 and most preferably amino acid position 51. Particularly, suitable regions for modifying the other amino acid within the alpha2 domain are within amino acid positions 165 to 178, preferably within amino acid positions 170 to 177, more preferably within amino acid positions 173 to 176 and most preferably amino acid position 175. Thus, in a particularly preferred embodiment the stabilized MHC I comprises a first pair of modified amino acids at position 22 and 71 and a second pair of modified amino acid at position 51 and at position 175.

Any of above modifications of MHC I may further be combined with a pair of modifications wherein the first modified amino acid is within amino acid positions 80 to 90,preferably within amino acid positions 82 to 86, and more preferably amino acid position 84 and the second amino acid is within amino acid positions 136 to 146, preferably within amino acid positions 137 to 141, and more preferably amino acid position 139.

It was surprising that the modification of amino acids in the above-described amino acid regions of MHC I and MHC II and at the respectively indicated positions and, thus the introduction of covalent bonds between amino acids at position which are not naturally connected by covalent bonds allows the generation of modified MHC I and MHC Il molecules that: (i) are properly folded, (ii) bind peptides with high affinity and (iii) are recognized by TCR molecules with high specificity and selectivity.

The preferred modified MHC I and MHC II molecules of the second aspect can also be used in all other aspects of the present invention.

The present invention also comprises peptide binding fragments of the modified MHC I or MHC Il molecules. As known in the art, MHC I and MHC II bind to peptides and are in turn bound by TCRs that interact both with the MHC I or MHC II and the peptide. However, only parts of the MHC I and MHC II molecule are required for binding to the peptide that is “presented” to the TCR. In MHC I the alpha1 and alpha2 domain fold to form a binding groove that binds the peptide and in MCH II the alpha1 and beta 1 domain form the binding groove that binds the peptide. Thus, peptide binding fragments of MHC I and MHC II, respectively, comprise at least the alpha1 and alpha2 domain or the alpha1 and beta1 domain. Accordingly, the binding fragment may lack the transmembrane domain or additionally the alpha3 domain in MHC I or the alpha2 and/or beta2 domain in MHC II. Fragments lacking at least the transmembrane domain are soluble and are particularly suitable to be used in a pharmaceutical composition, in particular in a vaccine.

In a third aspect thereof, the present invention provides a method for detecting or generating a specific amino acid binding motif for a TCR, comprising performing the method according to the first aspect thereof comprising a preselected TCR, and the additional step of determining and comparing the amino acid sequences of those peptide ligands in said peptide ligand/MHC molecule complexes for which a TCR binding was detected, thereby identifying the specific amino acid binding motif for said preselected TCR.

In a fourth aspect thereof, the present invention provides a method for detecting or determining cross-reactivity of a TCR, comprising performing the method according to the second aspect of the invention, and the additional step of determining and comparing the amino acid sequences of those peptide ligands in said peptide ligand/MHC molecule complexes for which a TCR binding was detected, thereby identifying cross-reactivity of said TCR.

In a fifth aspect thereof, the present invention provides a method for detecting or determining cross-reactivity of a TCR, comprising performing the method according to the first aspect of the invention comprising a preselected TCR, and the additional step of determining and comparing the amino acid sequences of those peptide ligands in said peptide ligand/MHC molecule complexes for which a TCR binding was detected, thereby identifying cross-reactivity of said TCR.

In a sixth aspect thereof, the methods according to the present invention can be used for screening or in vitro priming of cellular drug products. The stabilized HLA complexes bound to beads, filaments, nanoparticles or other carriers can be readily loaded with a peptide of interest mimicking antigen presenting cells, and afterwards conveniently used in combination with costimulatory molecules (e.g. anti CD28, anti 4 1BB) as “ready to use” artificial antigen presenting cells for in vitro priming and expansion.

Current methods for the large-scale generation of pMHC libraries, a high throughput binding motif determination of a high affinity TCR, and the identification and characterization of potentially cross-reactive peptides suffer from stability problems, requiring multimers to be swiftly loaded with peptides of choice without additional handling and within a short time frame (as in Luimstra et al., above), which also makes technologies like UV exchange unsuitable.

With the present technology, the inventors gain multiple advantages over the wild type molecule or other existing exchange technologies: the empty monomer can be produced in bulk way ahead of the desired experiment and pMHC generation is not restricted by any other method aside from procuring desired peptides and quick peptide loading reactions. The inventors have successfully stored the empty monomer for at least a year at −80° C. and used them with no degradation or impaired peptide receptiveness detected. The inventors have also successfully stored the resulting pMHC complexes for at least two weeks at 4° C. and reused them for affinity measurements without loss of signal. In addition to all these advantages achieved by introducing the modification, pMHC complexes generated displayed by the mutant are substantially representative of wild type complexes with respect to TCR ligand binding.

In one aspect, the invention provides a method for screening for a TCR-binding peptide ligand/MHC molecule complex for TCR-binding.

The method comprises the use of a suitably stabilized MHC molecule that comprises at least one artificially introduced covalent bridge between amino acids of the alpha1 domain and amino acids of the alpha2 domain of said stabilized MHC molecule in case of MHC I, and/or at least one artificially introduced covalent bridge between two amino acids of the alpha1 domain of said stabilized MHC molecule in case of MHC I, or at least one artificially introduced covalent bridge between amino acids of the alpha1 domain and amino acids of the beta1 domain of said stabilized MHC molecule in case of MHC II. Major histocompatibility complex class I and class Il share an overall similar fold. The binding platform is composed of two domains, originating from a single heavy α-chain (HC) in the case of MHC class I and from two chains in the case of MHC class II (α-chain and β-chain). The two domains evolved to form a slightly curved β-sheet as a base and two α-helices on top, which are far enough apart to accommodate a peptide chain in-between. Hence, suitable stabilization for the method according to the present invention can be achieved for both MHC classes.

In one embodiment, the present invention involves the use of disulfide-stabilized, initially empty, MHC molecules that can be loaded by simply adding suitable peptide before the use thereof. pMHCs generated using this disulfide-modified MHC molecule are representative of the non-modified wild type variant, and are suitable for screening, e.g. high throughput binding motif determination of a high affinity TCR as well as identification and characterization of potentially cross-reactive peptides.

The empty MHCs do not substantially degrade on commonly used surfaces, like glass plates, are representative for the non-modified wild type variant when loaded with peptide, and are suitable for screening, and allow to generate pMHCs quickly, even when immobilized on a surface. In the context of the present invention, this is achieved by and understood as a “suitably stabilized” or “stabilized” pMHC.

In previous studies with the murine MHC class I molecule H-2K^{b} introduction of a disulfide bond between opposing residues in the F-pocket by mutating a tyrosine at position 84 and an alanine at position 139 to cysteines was able to stabilize the complex. Thus, in one embodiment an artificially introduced covalent bridge between amino acids was introduced between α-helices, for example by mutating a tyrosine at position 84 and an alanine at position 139 into cysteines of MHC I. While in some cases, it may be difficult to isolate monomers without any peptide ligand, this could be efficiently overcome by adding a low affinity peptide.

The term “MHC” is an abbreviation for the phrase “major histocompatibility complex”. MHC's are a set of cell surface receptors that have an essential role in establishing acquired immunity against altered natural or foreign proteins in vertebrates, which in turn determines histocompatibility within a tissue. The main function of MHC molecules is to bind to antigens derived from altered proteins or pathogens and display them on the cell surface for recognition by appropriate T-cells. The human MHC is also called HLA (human leukocyte antigen) complex or HLA. The MHC gene family is divided into three subgroups: class I, class II, and class III. Complexes of peptide and MHC class I are recognized by CD8-positive T-cells bearing the appropriate TCR, whereas complexes of peptide and MHC class Il molecules are recognized by CD4-positive-helper-T-cells bearing the appropriate TCR. Since both types of response, CD8 and CD4 dependent, contribute jointly and synergistically to the anti-tumor effect, the identification and characterization of tumor-associated antigens and corresponding TCRs is important in the development of cancer immunotherapies such as vaccines and cell therapies. The MHC I molecule consists of an alpha chain, also referred to as MHC I heavy chain and a beta chain, which constitutes a beta 2 microglobulin molecule. The alpha chain, interchangeably used with heavy chain in the context of the present invention, comprises three alpha domains, i.e. alpha1 domain, alpha2 domain and alpha3 domain. Alpha1 and alpha2 domain mainly contribute to forming the peptide pocket to produce a peptide ligand MHC (pMHC) complex. The alpha1 domain of a MHC I spans amino acid positions 1 to 90 and comprises as secondary structure a β-sheet spanning amino acid positions 1-49 (termed herein “β1 unit”) followed by an α-helix structure spanning amino acid positions 50-84 (termed herein “α1 unit”). The alpha2 domain of a MHC I spans amino acid positions 91 to 182 and comprises as secondary structure a β-sheet spanning amino acid positions 91-135 (termed herein “β2 unit”) followed by an α-helix structure spanning amino acid positions 138-179 (termed herein “α2 unit”). The beta1 domain of a MHC II is on a separate polypeptide and fulfills within MHC II the structural role of the alpha2 domain of MHC I. It spans amino acid positions 1 to 95 and comprises as secondary structure β-sheet spanning amino acid positions 1 to 49 (termed herein “β3 unit”) followed by an α-helix structure spanning amino acid positions 50 to 95 (termed “α3 unit”). Here and in each other case in which reference is made to an amino acid position in an MHC I or MHC II molecule the positions are indicated based on IGMT numbering excluding the N-terminal first signal peptide, which typically varies in length between 20 to 29 amino acids. The stabilized MHC II molecules of the present invention may comprise the alpha1 and beta1 domain on two separate polypeptides or they may be linked to each other in one polypeptide to form a single chain MHC II, e.g. the C-terminus of the alpha1 domain of an MHC II is linked to the N-terminus of the beta1 domain of an MHC II directly or via a peptide linker.

HLAs are molecules which differ between different human beings in amino acid sequence. However, HLAs can be identified by an internationally agreed nomenclature, the IMGT nomenclature, of HLA. The categorization to, e.g. HLA-A, is based on the identity of a given HLA to official reference sequences of each HLA, that were produced by sequence alignments. Thus, a given HLA sequence with the highest sequence identity to the HLA-A sequence according to SEQ ID NO: 6 will be categorized as HLA-A. The official HLA reference sequences as well as information to the categorization system are available: www.ebi.ac.uk/ipd/imgt/hla/nomenclature/alignments.html. The website provides the following information regarding how to categorize any given HLA sequence:

“The alignment files produced use the following nomenclature and numbering conventions. These conventions are based on the recommendations published for Human Gene Mutations. These were prepared by a nomenclature-working group looking at how to name and store sequences for human allelic variants. These recommendations can be found in Antonarakis SE and the Nomenclature Working Group Recommendations for a Nomenclature System for Human Gene Mutations Human Mutation (1998) 11 1-3.
Only alleles officially recognised by the WHO HLA Nomenclature Committee for Factors of the HLA System are included in the sequence alignments.
As recommended for all human gene mutations, a standard reference sequence should be used for all alignments. A complete list of reference sequences for each allele can be seen below.
The reference sequence will always be associated with the same (original) accession number, unless this sequence is shown to be in error.
All alleles are aligned to the reference sequences.
Naming of the sequence is based upon the published naming conventions SGE Marsh, et al. (2010) Tissue Antigens 2010 75:291-455.”

For MHC class I proteins the following HLA reference protein sequences are indicated on Jul. 12, 2019 on the web site in each case indicating the accession number that will not change for each HLA over time:

HLA-A (Acc. No. HLA00001)
(SEQ ID NO: 6)
MAVMAPRTLLLLLSGALALTQTWAGSHSMRYFFTSVSRPGRGEPRFIAVGYVDDTQF
VRFDSDAASQKMEPRAPWIEQEGPEYWDQETRNMKAHSQTDRANLGTLRGYYNQS
EDGSHTIQIMYGCDVGPDGRFLRGYRQDAYDGKDYIALNEDLRSWTAADMAAQITKR
KWEAVHAAEQRRVYLEGRCVDGLRRYLENGKETLQRTDPPKTHMTHHPISDHEATLR
CWALGFYPAEITLTWQRDGEDQTQDTELVETRPAGDGTFQKWAAVVVPSGEEQRYT
CHVQHEGLPKPLTLRWELSSQPTIPIVGIIAGLVLLGAVITGAVVAAVMWRRKSSDRKG
GSYTQAASSDSAQGSDVSLTACKV
HLA-B (Acc. No. HLA00132)
(SEQ ID NO: 7)
MLVMAPRTVLLLLSAALALTETWAGSHSMRYFYTSVSRPGRGEPRFISVGYVDDTQFV
RFDSDAASPREEPRAPWIEQEGPEYWDRNTQIYKAQAQTDRESLRNLRGYYNQSEA
GSHTLQSMYGCDVGPDGRLLRGHDQYAYDGKDYIALNEDLRSWTAADTAAQITQRK
WEAAREAEQRRAYLEGECVEWLRRYLENGKDKLERADPPKTHVTHHPISDHEATLRC
WALGFYPAEITLTWQRDGEDQTQDTELVETRPAGDRTFQKWAAVVVPSGEEQRYTC
HVQHEGLPKPLTLRWEPSSQSTVPIVGIVAGLAVLAVVVIGAVVAAVMCRRKSSGGKG
GSYSQAACSDSAQGSDVSLTA
HLA-C (Acc. No. HLA00401)
(SEQ ID NO: 8)
MRVMAPRTLILLLSGALALTETWACSHSMKYFFTSVSRPGRGEPRFISVGYVDDTQFV
RFDSDAASPRGEPRAPWVEQEGPEYWDRETQKYKRQAQTDRVSLRNLRGYYNQSE
AGSHTLQWMCGCDLGPDGRLLRGYDQYAYDGKDYIALNEDLRSWTAADTAAQITQR
KWEAAREAEQRRAYLEGTCVEWLRRYLENGKETLQRAEHPKTHVTHHPVSDHEATL
RCWALGFYPAEITLTWQWDGEDQTQDTELVETRPAGDGTFQKWAAVMVPSGEEQR
YTCHVQHEGLPEPLTLRWEPSSQPTIPIVGIVAGLAVLAVLAVLGAVVAVVMCRRKSSG
GKGGSCSQAASSNSAQGSDESLIACKA
HLA-E (Acc. No. HLA00934)
(SEQ ID NO: 9)
MVDGTLLLLLSEALALTQTWAGSHSLKYFHTSVSRPGRGEPRFISVGYVDDTQFVRFD
NDAASPRMVPRAPWMEQEGSEYWDRETRSARDTAQIFRVNLRTLRGYYNQSEAGSH
TLQWMHGCELGPDRRFLRGYEQFAYDGKDYLTLNEDLRSWTAVDTAAQISEQKSND
ASEAEHQRAYLEDTCVEWLHKYLEKGKETLLHLEPPKTHVTHHPISDHEATLRCWALG
FYPAEITLTWQQDGEGHTQDTELVETRPAGDGTFQKWAAVVVPSGEEQRYTCHVQH
EGLPEPVTLRWKPASQPTIPIVGIIAGLVLLGSVVSGAVVAAVIWRKKSSGGKGGSYSK
AEWSDSAQGSESHSL
HLA-F (Acc. No. HLA01096)
(SEQ ID NO: 10)
MAPRSLLLLLSGALALTDTWAGSHSLRYFSTAVSRPGRGEPRYIAVEYVDDTQFLRFD
SDAAIPRMEPREPWVEQEGPQYWEWTTGYAKANAQTDRVALRNLLRRYNQSEAGSH
TLQGMNGCDMGPDGRLLRGYHQHAYDGKDYISLNEDLRSWTAADTVAQITQRFYEAE
EYAEEFRTYLEGECLELLRRYLENGKETLQRADPPKAHVAHHPISDHEATLRCWALGF
YPAEITLTWQRDGEEQTQDTELVETRPAGDGTFQKWAAVVVPSGEEQRYTCHVQHE
GLPQPLILRWEQSPQPTIPIVGIVAGLVVLGAVVTGAVVAAVMWRKKSSDRNRGSYSQ
AAV
HLA-G (Acc. No. HLA00939)
(SEQ ID NO: 11)
MVVMAPRTLFLLLSGALTLTETWAGSHSMRYFSAAVSRPGRGEPRFIAMGYVDDTQF
VRFDSDSACPRMEPRAPWVEQEGPEYWEEETRNTKAHAQTDRMNLQTLRGYYNQS
EASSHTLQWMIGCDLGSDGRLLRGYEQYAYDGKDYLALNEDLRSWTAADTAAQISKR
KCEAANVAEQRRAYLEGTCVEWLHRYLENGKEMLQRADPPKTHVTHHPVFDYEATLR
CWALGFYPAEIILTWQRDGEDQTQDVELVETRPAGDGTFQKWAAVVVPSGEEQRYTC
HVQHEGLPEPLMLRWKQSSLPTIPIMGIVAGLVVLAAVVTGAAVAAVLWRKKSSD
HLA-H (Acc. No. HLA02546)
(SEQ ID NO: 12)
MVLMAPRTLLLLLSGALALTQTWARSHSMRYFYTTMSRPGRGEPRFISVGYVDDTQF
VRFDSDAASQRMEPRAPWMEREGPEYWDRNTQICKAQAQTERENLRIALRYYNQSE
GGSHTMQVMYGCDVGPDGRFLRGYEQHAYDSKDYIALNEDLRSWTAADMAAQITKR
KWEAARQAEQLRAYLEGEFVEWLRRYLENGKETLQRADPPKTHMTHHPISDHEATLR
CWALGFYPAEITLTWQRDGEDQTHTRSSWRPGLQGMEPSRSGRLWWCLLERSRDT
PAMCSMRVCQSPSP*DGSHLPSPPSPSWASLLAWFYL*LWSLELWSLL*CGGRRAQIE
KEGATLRLQAATVPRALMCLSRRESVX
HLA-J (Acc. No. HLA02626)
(SEQ ID NO: 13)
MGSWRPEPSSCCSRGPWPWPRPGRAPTP*GISAPPFPGRAAGSPASLPWATWTTRS
SCGSTVTP*V*G*RRGRGGWSRRGRSIGTYRHWAPRPRHRLTE*TCGPCSATTTRAR
RGITSSRECLAATWGPTGVSSAGMSSMPTTARITSP*TRTCAPGPPRIPRLRLPSASM
RRPMWLSKGEPTWRAPAWSGSADTWRTGRRRCSARTPPKTHVTHPPL*T*GITRSW
VLGFYPAEITLTWQRDGEDQTQDMELVETRPTGDGTFQKWAVVVVPSGEEQRYTCH
VQHKGLPKPLILRWEPSPQPTIPIVGIIAGLVLLGAVVTGAVVTAVMWRKKSSDRKGGS
YSQAASSQSAQGSDVSLTACKV*
HLA-K (Acc. No. HLA02654)
(SEQ ID NO: 14)
MGSWRPEPSSCCSWGPWP*PRPGRVPTP*GISAPPCPGRVAGSPGTSQWATWTTR
SSCGSTATRRLRGCSRSRRGWSRRDRSIGTGAHGTSGPRTD*QE*TCPCRAATTTRA
RPGLTPSR*CMAATWGWKGASSAGMNSTPTMARIT*PGTRTCAPGPRRTWRLRSPS
ASGRQKNLQSRSGPTWRARAWRGSQTPGEREGDAAAHGPLPQTHMIHHSVSDYKA
TLRCWALGFYPVEITLAWQQDGEDQTRDMELLETRPAGDGTFQKWAAVVVPSGEEQ
RYPCHVQHEGLPKPLTLRWEQSSQPTIPIVGIVAGLVLLGAVVTGAVVSAVMCRKKNS
DRVSYSEAASSDHAQGSDVSLTACKV*
HLA-L (Acc. No. HLA02655)
(SEQ ID NO: 15)
MGVMAPRTLLLLLLGALALTETWAGSHSLRYFSTAVSQPGRGEPRFIAVGYVDDTEFV
RFDSDSVSPRMERRAPWVEQEGLEYWDQETRNAKGHAQIYRVNLRTLLRYYNQSEA
GSHTIQRKHGCDVGPTGASSAGMNSSPTMARITSP*TRTCTPGPPRTQRLRSPSTSG
KRTNTQSRSGPT*GQVHGVAPQTPGEREGDAAARGSPKGTCDPAPHL*P*GHPEVLG
PGPLPCGDHTDLAAGWGGPDPGHGACGDQACRGRNLPEVGGCSGAFRRGAEIHVP
CAA*GAARAPHPEMGAVFSAHHPHRGHRCWPVSPWSCGHWSCGCCCDVEEEKLR*
NKEELCSGCLQQLCSVL*CIS*YL*SLX

The HLA-A gene is located on the short arm of chromosome 6 and encodes the larger, α-chain, constituent of HLA-A. Variation of HLA-A α-chain is key to HLA function. This variation promotes genetic diversity in the population. Since each HLA has a different affinity for peptides of certain structures, greater variety of HLAs means greater variety of antigens to be ‘presented’ on the cell surface. Each individual can express up to two types of HLA-A, one from each of their parents. Some individuals will inherit the same HLA-A from both parents, decreasing their individual HLA diversity. However, the majority of individuals receive two different copies of HLA-A. The same pattern follows for all HLA groups. In other words, every single person can only express either one or two of the 2432 known HLA-A alleles coding for currently 1740 active proteins. HLA-A*02 signifies a specific HLA allele, wherein the letter A signifies to which HLA gene the allele belongs to and the prefix “*02 prefix” indicates the A2 serotype. In MHC class I dependent immune reactions, peptides not only have to be able to bind to certain MHC class I molecules expressed by tumor cells, they subsequently also have to be recognized by T-cells bearing specific TCRs.

In the second step of the preferred method according to the invention, the suitably stabilized MHC molecule is contacted with a multitude of peptide ligands, in order to form peptide ligand/MHC (pMHC) molecule complexes. Using pMHC complexes as soluble analytes instead of immobilizing is preferable for quick and cost-effective high throughput screenings, since a broad variety of regeneratable biosensors capable of reversibly immobilizing bispecific TCR constructs exists.

“Contacting” in the context of the present invention shall mean that peptide(s) is (are) brought in contact with the empty and/or low affinity peptide-loaded MHC molecules in such a way that a substantial portion of the peptides form complexes (are “loaded”) with said empty and/or low affinity peptide-loaded MHC molecules. As one preferred example, loading MHC complexes was performed by addition and mixing of desired peptides of at least a 100 to 1 molar ratio to the monomer solution in a suitable buffer, and a minimum of 5 minutes incubation at room temperature.

The groove in-between the two helices accommodates peptides based on (i) the formation of a set of conserved hydrogen bonds between the side-chains of the MHC molecule and the backbone of the peptide and (ii) the occupation of defined pockets by peptide side chains (anchor residues P2 or P5/6 and PΩ in MHC class I and P1, P4, P6, and P9 in MHC class II). The type of interactions of individual peptide side-chains with the MHC depend on the geometry, charge distribution, and hydrophobicity of the binding groove. In MHC class I, the binding groove is closed at both ends by conserved tyrosine residues leading to a size restriction of the bound peptides to usually 8-10 residues with its C-terminal end docking into the F-pocket. In contrast, MHC class II proteins usually accommodate peptides of 13-25 residues in length in their open binding groove, with the peptide N-terminus usually extruding from the P1 pocket. It has been reported that the interactions at the F pocket region in MHC class I and the P1 region (including the P2 site) in MHC class II appear to have a dominant effect on the presentation of stable pMHC complexes and on the immunodominance of certain peptidic epitopes. Interestingly, these pockets are located at opposite ends of the binding groove of the respective MHC class I and MHC class II structures.

The multitude of peptide ligands can comprise at least about 1,500 different MHC binding peptides, preferably at least about 5,000 different MHC binding peptides, more preferred at least about 15,000 different MHC binding peptides, and most preferred an immunopeptidome preparation with at least about 150,000 MHC binding peptides. Said peptides comprise a binding motif of 8-10 residues in length for MHC class I proteins and 13-25 residues in length for MHC class II proteins, and can be of a length of between 8 and 100, preferably of between 8 and 30, more preferred between 8 and 16 residues. Most preferred are peptides that consist of the actual binding motif.

Ligand peptides as used in the context of the present invention can be derived from polypeptides that are cancer-related, infection-related (bacterial or viral), and even immune- (e.g. autoimmune-) disease related. The term also includes suitably mutated or naturally occurring mutated ligand peptides, i.e. different from their underlying sequence as occurring in the respective polypeptide.

Preferred is the method according to the present invention, wherein said contacting comprises loading said MHC binding peptides onto the MHC at between about 4° C. to 37° C., preferably at about room temperature (15° to 25° C., preferably 20° C.).

It was surprisingly found that the loaded HLA/peptide molecules (pMHC or pMHC complex) are very stable for more than about 1 day, and preferably for more than 1 week at (e.g. more than 2 weeks) at about 4° C. This allows an effective and convenient use in many more applications than in known methods as described above.

It was also found in the context of the present invention, and somewhat in contrast to the literature as above, that the present method was clearly superior to the popular method of UV exchange using a WT pMHC molecule, allowing to perform it (in particular in a high-throughput format) on a surface, like a chip or glass slide. While the UV mediated peptide ligand exchange can generate a high number of different pMHC complexes, the exchange efficiency varies depending on the peptide and its affinity for binding to the respective MHC class I allele, resulting in different pMHC concentrations in the samples. This uncertainty is a problem for affinity measurements with pMHCs used as soluble analytes, as precise knowledge of the concentration is required to determine accurate affinities. Since the disulfide-stabilized MHC mutant is stable without peptide, this restriction does not apply. If the peptides are added at a concentration high enough to saturate the empty MHC complexes, the effective concentration of pMHC is known, significantly increasing the accuracy of the measurements and avoiding false negatives.

In the next step of the method of the present invention, said pMHC molecule complexes are screened for a TCR-binding. The binding and kinetic attributes of this interaction are parameters for protective T cell-mediated immunity, with stronger TCR-pMHC interactions showing increased interaction half-life and thus conferring superior T cell activation and responsiveness than weaker ones. The interaction strength between the TCR and pMHC ligand is typically described and measured as the dissociation constant K_{d}, an equilibrium constant that is a ratio between the on-rate constant kₒₙ and off-rate constant K_{off} of a specific interaction. The dissociation constant K_{d} inversely correlates with the binding strength of a specific interaction, as smaller K_{d} values represent stronger binding.

The screening can comprise any suitable and known method for measuring and/or detecting pMHC/TCR-binding, e.g. structural TCR-pMHC affinity/avidity measurements. One example is screening of a peptide-MHC library for TCR binding by bio-layer interferometry (BLI), a special form of reflective interferometry (RI), as disclosed herein, where binding interactions for said TCR were detected stronger than a sensitivity threshold suitable for the method of K_{d} 1.0×10−5, with measured K_{d} values ranging from 3.7×10⁻⁹ to 7.2×10⁻⁶, or no binding interactions for said TCR were detected when weaker than the sensitivity threshold.

Other methods involve other forms of RI, like surface plasmon resonance (SPR), or reflective interferometric spectroscopy (RIfS), or single-color reflectometry (SCORE, Biametrics, Tübingen, Germany), or marker-based assays, e.g. flow cytometric analysis with NTAmers (TCMetrix, Epalinges, Switzerland), or pMHC or TCR tetramers, or other forms of fluorescent readouts, like protein microarrays. Of course, ideally these methods can be performed in/can be readily adjusted to high-throughput formats.

In the context of the present invention, the term “about” shall mean to include +/−10% of a given value, unless otherwise noted.

The present invention as an example presents the use of disulfide-stabilized empty HLA-A*02:01 molecules which can be loaded by simply adding peptide before use. pMHCs generated using this modified MHC molecule are representative of the non-modified wild type variant and thus, demonstrate suitability for high throughput binding motif determination of a high affinity TCR as well as identification and characterization of potentially cross-reactive peptides.

Preferred is a method according to the present invention, wherein said MHC molecule is HLA, or a multimer of HLA, MHC I or MHC II, selected from the group consisting of a dimer, a trimer and a tetramer. Methods using more than one MHC molecule at once in screenings are known in the art, e.g. from Altman, et al. (in: “Phenotypic Analysis of Antigen-Specific T Lymphocytes.”, Science. 4 Oct. 1996: Vol. 274, Issue 5284, pp. 94-969. Similarly, dimers or trimers can be used.

The MHC molecules as used include at least one artificially introduced covalent bridge between amino acids. This bridge is selected from a recombinantly introduced disulfide bridge, the introduction of non-natural amino acids to be crosslinked, the introduction of photo-crosslinking amino acids, and chemically introduced crosslinks. The introduction of crosslinks using cysteines is described herein; examples for dimeric cross-linking reagents are DPDPB and HBVS, and the trimeric cross-linker TMEA.

Preferred is a method according to the present invention, wherein said at least one artificially introduced covalent bridge between amino acids is introduced between α-helices, for example by i) mutating the amino acid at position 84 of MHC I, a tyrosine in the majority of HLAS (see FIG. 13) and an amino acid at position 139, a alanine in the majority of HLAs (see FIG. 13) into cysteines and/or (ii) mutating an amino acid at position 22 of MHC I, a phenylalanine in the majority of HLAs (see FIG. 13) and an amino acid at position 71 of MHC I, a serine in the majority of HLAS (see FIG. 13) and/or (iii) mutating an amino acid at position 51 of MHC I, a tryptophan in the majority of HLAs (see FIG. 13), and an amino acid at position 175 of MHC I, a glycine in the majority of HLAS (see FIG. 13), or (iv) mutating an amino acid at position 22 of MHC I, a phenylalanine in the majority of HLAs (see FIG. 13) and an amino acid at position 71 of MHC I, a serine in the majority of HLAS (see FIG. 13) and mutating an amino acid at position 51 of MHC I, a tryptophan in the majority of HLAs (see FIG. 13), and an amino acid at position 175 of MHC I, a glycine in the majority of HLAS (see FIG. 13) of MHC I (based on IGMT numbering excluding the first 24 amino acids). Molecular dynamics simulations of the α₁ and α₂ domain or of entire MHC-I have suggested one eminent difference between empty and peptide-bound MHC-I: in the absence of a peptide, the helical sections that flank the F-pocket region (residues 74-85 and 138-149 in the α₁ and α₂ helices, respectively) are significantly more mobile. It seems that bound peptides restrict the mobility of this region, and that a similar advantageous and stabilizing conformational restriction might be achieved by linking different structural features of the peptide binding pocket with a covalent bond, preferably a disulfide bond.

To determine amino acids at positions corresponding to above mentioned residues 22, 51, 71, 74-85, 138-149 and 175 in each given HLA allele the respective sequence is aligned with the above indicated reference antibodies. An example of the alignment of multiple sequences of official HLA (MHC class I) reference protein sequences and murine MHC I H2Kb protein; highlighting amino acid positions 22, 51, 71, 84, 85, 139, 140 and 175 (bold) and further regions suitable for introducing stabilizing mutations (grey) is shown in FIGS. 13A and 13B. FIGS. 13A and 13B will enable the skilled person to identify the amino acids at positions corresponding to above mentioned residues 22, 51, 71, 74-85, 138-149 and 175 in each given HLA allele.

In one preferred embodiment the MHC I molecule used in the present invention is a MHC class I HLA protein, preferably HLA-A, HLA-B, HLA-C, HLA-E, HLA-F, HLA-G, HLA-H, HLA-J, HLA-K, HLA-L. These preferred HLA proteins can be mutated in their α₁ domain and α₂ domain, respectively, according to the reference sequences of the IMGT nomenclature. Preferably, these HLA proteins are mutated at one or more, preferably one amino acid within position 22; at one or more, preferably one amino acid within position 51, at one or more, preferably one amino acid within position 71, at one or more, preferably one amino acid within positions 74-85, at one or more, preferably one amino acid within positions 138-149, and at one or more, preferably one amino acid within position 175. Even more preferably, one amino acid is mutated at position 84 or 85 and one amino acid is mutated at position 139 or 140. Even more preferably, one amino acid is mutated at position 22 and one amino acid is mutated at position 71. Even more preferably, one amino acid is mutated at position 22 and one amino acid is mutated at position 71 and one amino acid is mutated at position 51 and one amino acid is mutated at position 175. Preferred amino acid mutations are substitutions of one amino acid at positions 74-85 and one amino acid at positions 138-149 to cysteine. Even more preferred amino acid mutations are substitutions of one amino acid at positions 22 to cysteine. Even more preferred amino acid mutations are substitutions of one amino acid at positions 51 to cysteine. Even more preferred amino acid mutations are substitutions of one amino acid at positions 71 to cysteine. Even more preferred amino acid mutations are substitutions of one amino acid at positions 175 to cysteine.

In another preferred embodiment the HLA-A protein is selected from the group consisting of HLA-A1, HLA-A2, HLA-A3, and HLA-A11. These preferred HLA-A proteins can be mutated in their α₁ domain and α₂ domain, respectively, according to the reference sequences of the IMGT nomenclature. Preferably these HLA proteins can be mutated at amino acid positions 22,51, 74-85, 138-149 and amino acid position 175. It is even more preferably that the HLA-A protein is a HLA-A*02 protein. Preferred HLA-A alleles are HLA-A*02:01; HLA-A*01:01 or HLA-A*03:01.

In another preferred embodiment the HLA-B protein is selected from the group consisting of HLA-B*07, HLA-B*08, HLA-B*15, HLA-B*35 or HLA-B*44. These preferred HLA-B proteins can be mutated in their α₁ domain and α₂ domain, respectively, according to the reference sequences of the IMGT nomenclature. Preferably these HLB proteins can be mutated at amino acid positions 74-85 and amino acid positions 138-149. Preferred HLA-B alleles are HLA-B*07:02; HLA-B*08:01, HLA-B*15:01, HLA-B*35:01 or HLA-B*44:05.

In the context of the present invention, the term “TCR” shall include any proteinaceous molecule/construct that comprises a TCR-derived or TCR-like binding domain, wherein the molecule/construct is suitable for the analysis/detection of pMHC/TCR binding according to the invention as described herein. In the case of the α- and/or β-chain of a TCR, this may include a molecule where both chains remain able to form a T-cell receptor (either with a non-modified α- and/or β-chain or with a fusion protein or modified α- and/or β-chain) which exerts its biological function, in particular binding to a (specific) pMHC, and/or functional signal transduction upon peptide activation. Preferred is a method according to the present invention, wherein said TCR is selected from a native TCR, a soluble TCR molecule, a single-chain TCR, and a TCR-like molecules comprising a TCR-derived or TCR-like binding domain (e.g. derived from an antibody), such as a bispecific (bs) TCR, for example like the ones as described herein.

The methods according to the present invention in preferred embodiments allow for a parallel detection, analysis and/or screening of a much larger number of peptide ligands and/or pMHC, when compared to common technologies, including UV exchange-related methods. The collection of peptides presented to the cell surface by class I and class II human leukocyte antigen (HLA) molecules are referred to as the immunopeptidome. In May 2017, already 119,073 high-confidence HLA class I peptides and 73,465 high-confidence HLA class II peptides were reported (Shao W, Pedrioli PGA, Wolski W, et al. The SysteMHC Atlas project. Nucleic Acids Research. 2018;46 (Database issue): D1237-D1247), and therefore it can be expected that the human immunopeptidome exceeds 150,000 MHC binding peptides for each of class I and II. Current methods can analyze about 700 peptides a day, so that there is a demand for “true” high throughput methods, i.e. a multitude of peptide ligands as analyzed that comprises at least about 1,500 different MHC binding peptides, preferably at least about 5,000 different MHC binding peptides, more preferred at least about 15,000 different MHC binding peptides, and most preferred a substantially complete immunopeptidome preparation with at least about 150,000 MHC binding peptides.

The inventive methods allow for immunopeptidome-wide screening for as short of a period as within a day.

In view of the number of pMHC/TCR bindings to be detected/analyzed, preferred is a method according to the present invention, wherein said method is performed as a high-throughput screening (HTS) format. In HTS, up to hundreds of thousands of experimental samples can be subjected to simultaneous testing for pMHC/TCR binding under given conditions. The samples are usually and preferably handled by laboratory robotics that automate sample preparation, handling and data analysis. HTS thus easily and reliably generates and uses large datasets to answer complex biological questions, e.g. pMHC/TCR binding kinetics and biological function as described herein.

HTS classically requires samples to be prepared in an arrayed format. If necessary, the arrayed samples can be grown either on microtiter plates in liquid, or on solid agar. The density of plates can range from 96, 192, 384, 768, 1,536, or 6, 144. All these densities are multiples of 96,reflecting the original 96-well microtiter plate arranged in 8×12 with 9 mm spacing (see also, for example, Bean GJ, Jaeger PA, Bahr S, Ideker T. “Development of Ultra-High-Density Screening Tools for Microbial “Omics.”” PLOS ONE. 2014 Jan. 21;9(1):e85177).

For uses relating to pMHC/TCR binding kinetics as detected/analyzed and as described herein, a solid surface, such as a chip, biosensor, glass slide or bead can be used, onto which some of the analysis reagents (e.g. either the TCR or the MHC molecule) can be suitably immobilized, e.g. spotted. For immobilization, any suitable technique can be used, e.g. by biotin streptavidin interaction. Examples of the embodiments as described here are binding assays involving binding of at least one soluble TCR(s) against at least one immobilized pMHC(s), or binding of at least one immobilized TCR(s) against at least one soluble pMHC(s).

Preferred is the method according to the present invention, wherein said TCR and/or the MHC molecule is/are not labelled or suitably labelled with a detectable marker. Respective markers are known in the art and include direct or indirect labelling with radioactive, fluorescent or chemical groups (e.g. dyes). Also, enzymatic markers or antigenic markers (for a detection with antibodies) as well as mass markers can be used. Another option is coding markers (e.g. specific nucleic acids). In case of no labelling, a detection of the binding based on changes in the physical state upon complex formation/binding can be used in order to identify binding, such as a change in mass, charge, or changes in optical properties, for example of the optical thickness of the biolayer by analyte binding, and thus of the interference pattern or reflection coefficient.

Methods to detect a binding, in particular a “specific” binding of a pMHC with a TCR are known in the art. In the present invention, preferred is a method according to the present invention, wherein K_{d} values as well as kₒₙ and K_{off} values can be measured for said TCR, preferably with sensitivity between K_{d}s of 1×10⁻¹⁰ M and 1×10⁻³ M, where sensitivity can be directed by analyte concentration.

As one preferred example, the affinity is measured using 1:2 analyte dilution series starting at 500 nM, or using 1/\sqrt{10} analyte dilution series starting at 500 nM. As one preferred example, the peptide ligand/MHC molecule complexes are used in parallel assay reactions having different concentrations.

In yet another important aspect of the method according to the present invention, said method further comprises the step of measuring T cell activation comprising a TCR and a TCR-binding peptide ligand/MHC molecule complex that binds said TCR. Methods to detect such T cell activation through a binding, in particular a “specific” binding of a pMHC to a TCR are known in the art. In the present invention, as an example, co-incubation assays with peptide loaded target cells, Jurkat effector cells and bs-868Z11-CD3 at six different concentrations were performed, and a correlation of measured affinity for the peptide ligands from the positional scanning library with the lowest bsTCR concentration necessary to induce 3-fold luminescence increase over background was taken as a cut-off.

Yet another important aspect of the invention is a method for detecting or generating a specific amino acid binding motif for a TCR, comprising performing the method according to the present invention as described herein, wherein a preselected TCR is chosen, for which a specific amino acid binding motif is to be detected or generated. The method comprises a) providing a suitably stabilized MHC molecule, wherein said MHC molecule comprises at least one artificially introduced covalent bridge between amino acids of the alpha1 domain and amino acids of the alpha2 domain of said stabilized MHC molecule in case of MHC I, and at least one artificially introduced covalent bridge between amino acids of the alpha1 domain and amino acids of the beta1 domain of said stabilized MHC molecule in case of MHC II, b) contacting said suitably stabilized MHC molecule with a multitude of peptide ligands thereof, to form peptide ligand/MHC (pMHC) molecule complexes, and c) screening said pMHC molecule complexes for TCR-binding using said pre-selected TCR. In an additional step, the amino acid sequences of those peptide ligands in said peptide ligand/MHC molecule complexes for which a TCR binding was detected are determined and optionally and preferably compared, resulting in identifying the specific amino acid binding motif for said preselected TCR.

One additional embodiment comprises a mutagenesis of a particular amino acid sequence after the identification thereof, and contacting said mutated peptides with a suitably stabilized MHC molecule, and screening said pMHC molecule complexes for TCR-binding with a preselected TCR to obtain an amino acid binding motif for said preselected TCR. The mutagenesis of peptides can easily be performed, for example by synthesizing mutated peptides, or chemically modifying existing amino acids in respective peptide binders. The mutagenesis can also involve adding markers or other groups to the peptide(s) in order to identify diagnostically effective binders. This aspect relates to the method according to the present invention as described herein, wherein said method steps are repeated comprising a pool of peptides consisting of modified amino acid sequences for said preselected TCR as identified. The modification can furthermore be guided by one of the known computer algorithms and/or programs used to calculate improved binding parameters based on modifications of the amino acid sequence(s).

One example thereof is the screening of a pMHC complex library, comprised of peptides created in said fashion, against a preselected TCR for TCR binding by bio-layer interferometry (BLI) as disclosed herein, where binding interactions for said TCR were detected stronger than a sensitivity threshold suitable for the method of K_{d} 1.0×10⁻⁵ M, with measured K_{d} values ranging from 3.7×10⁻⁹ M to 7.2×10⁻⁶ M, or no binding interactions for said TCR were detected when weaker than the sensitivity threshold. In said embodiment the present invention shows particular improvement over existing methods, as generation of pMHC complexes with a suitably stabilized MHC molecule generates predictable amounts of pMHC, thus increasing K_{d} measurement accuracy compared to existing methods (FIGS. 5A-5E).

In one additional embodiment, the multitude of peptide ligands is mostly composed of known peptide ligands from the immunopeptidome, as identified e.g. by mass spectrometry, wherein a preselected TCR is screened for TCR-binding to directly identify existing cross-reactive peptide ligands for said TCR. Preferred is the method according to this embodiment where the number of different peptides comprises at least about 1,500 different MHC binding peptides, preferably at least about 5,000 different MHC binding peptides that are measured in parallel.

Yet another important aspect of the invention is a method for detecting or determining cross-reactivity of a TCR, comprising performing the method for detecting or generating a specific amino acid binding motif for a TCR as described herein, and the additional step of determining and comparing the amino acid sequences of those peptide ligands in said peptide ligand/MHC molecule complexes for which a TCR binding was detected, thereby identifying cross-reactivity of said TCR. This aspect detects variants of a peptide that are recognized by a single TCR.

Yet another important aspect of the invention is a method for detecting or determining cross-reactivity of a TCR, comprising performing the method for screening for a TCR-binding peptide ligand/MHC molecule complex for TCR-binding according to the present invention as described herein comprising a preselected TCR, and the additional step of determining and comparing the amino acid sequences of those peptide ligands in said peptide ligand/MHC molecule complexes for which a TCR binding was detected, thereby identifying cross-reactivity of said TCR. This aspect also detects variants of a peptide that are recognized by a single TCR.

One example thereof is identification of a cross-reactive peptide ligand based on the amino acid binding motif, previously determined by screening a preselected TCR for TCR-binding with a mutagenesis derived pMHC complex library according to the present invention, and searching for a matching peptide ligand in a database of known or assumed peptide ligands.

Yet another important aspect of the invention is a method for detecting or determining cross-reactivity of a peptide ligand/MHC molecule complex, comprising performing the method for screening for a TCR-binding peptide ligand/MHC molecule complex for TCR-binding according to the present invention as described herein comprising a preselected pMHC, and the additional step of identifying of those TCRs for which a pMHC binding was detected, thereby identifying cross-reactivity of said TCR. This aspect detects variants of TCRs that recognize a single peptide.

In these aspects, the same methods to detect a binding of a pMHC with a preselected TCR can be used as above. Nevertheless, as the TCR binding is not necessarily required to be specific, the cut-off value and sensitivity for measuring and evaluating binding does not need to be optimal, and should be chosen as best suited under the respective circumstances, which will be comprehensible to a person of skill.

In another important aspect of the methods according to the present invention, said methods can further comprise the step of measuring T cell activation comprising a TCR and a TCR-binding peptide ligand/MHC molecule complex that binds said TCR. Methods to detect such T cell activation through a binding, in particular a “specific” binding of a pMHC to a TCR are known in the art. In the present invention, as an example, co-incubation assays with peptide loaded target cells, Jurkat effector cells and bs-868Z11-CD3 at six different concentrations were performed, and a correlation of measured affinity for the peptide ligands from the positional scanning library with the lowest bsTCR concentration necessary to induce 3-fold luminescence increase over background was taken as a cut-off.

Another important aspect of the present invention then relates to a pharmaceutical composition comprising a suitably stabilized MHC molecule, wherein said MHC molecule comprises at least one artificially introduced covalent bridge between amino acids of the alpha1 domain and amino acids of the alpha2 domain of said stabilized MHC molecule in case of MHC I, and/or at least one artificially introduced covalent bridge between two amino acids of the alpha1 domain of said stabilized MHC molecule in case of MHC I, and/or at least one artificially introduced covalent bridge between amino acids of the alpha1 domain and amino acids of the beta1 domain of said stabilized MHC molecule in case of MHC II, wherein said stabilized MHC molecule is bound to a bead, filament, nanoparticle or other suitable carrier.

In a preferred embodiment the pharmaceutical composition comprises a stabilized MHC molecule according to the second aspect of the invention as described above in the second aspect of the invention. Preferably, the stabilized MHC molecule comprised in the pharmaceutical composition does not comprise a transmembrane domain.

The pharmaceutical composition furthermore comprises suitable buffers and/or excipients. Preferably, said pharmaceutical composition according to the present invention can further comprise one or a combination of more and/or a chronological sequence of these costimulatory molecules, such as an anti CD28 or anti 41BB antibody.

Another important aspect of the present invention then relates to the use of the pharmaceutical composition according to the present invention in a method according to the invention as herein.

In one embodiment the pharmaceutical composition is comprised in a vaccine. In another embodiment the pharmaceutical composition is comprised in a vaccine for use in the manufacturing of a medicament. Preferably, the vaccine is used in the prevention of cancer. Even more preferably the vaccine elicits or triggers a subject's T cell response after administration to a subject in need thereof. Preferably, the stabilized MHC molecule comprised in the pharmaceutical composition in the vaccine does not comprise a transmembrane domain.

Another important aspect of the present invention then relates to a method for the improved personalized identification of T cell receptors, or activation of T-cells, and/or T-cell therapeutics against proliferative diseases, such as cancer, by stimulation with pMHC complexes to generate cellular drug products for a specific patient. Such stimulation can be based on pMHC complexes loaded with peptides identified by obtaining/providing a sample of cancer tissue and/or cancer cells from said patient, providing obtaining/providing a sample of normal tissue and/or cells from said patient, detecting peptides as presented in the context of MHC in said sample(s) using the XPRESIDENT® or comparable method, and determining the sequence(s) of at least one of said peptides, optionally, detecting the expression of the underlying genes of said peptides as determined, detecting the MHC presentation level/number of the peptides as detected in said sample(s), optionally comparing said MHC presentation level/number of the peptides as detected in said tumor and normal tissue and/or cell samples, screening for an optimized TCR-binding peptide ligand/MHC molecule complex, comprising a method according to the present invention. Said T-cells include those recovered directly from said patient which can be re-administered after said stimulation as cellular drug product. Said stimulation can include the use of preproduced stimulation frameworks, produced by immobilization of a suitably stabilized MHC molecule, preferably produced under clinical grade conditions (e.g. GMP), onto a carrier, for example filaments or beads, that are then loaded with peptide on demand, for example directly at the clinical site. These stimulation frameworks can also include other costimulatory molecules (e.g. anti CD28 antibodies, anti 41BB antibodies) immobilized together with the suitably stabilized MHC molecule.

In a preferred aspect of the above method said peptide, e.g. a peptide specific for a certain type of cancer or other kind of proliferative disease, is already known to the entity performing the procedure through previous identification in another patient or patients. Said peptide can thus be selected and produced quickly for a different patient bearing the same type of cancer, loaded on said stimulation framework and used to produce a cellular drug product.

In a preferred aspect of the above method, said process of activation of T-cells, and/or T-cell therapeutics recovered directly from said patient also comprises transducing the T-cells to express a tumor-specific exogenous T-cell receptor (TCR), and, optionally, suitably formulating said resulting T-cell therapeutic.

The term “T cell” refers to T lymphocytes as defined in the art and is intended to include recombinant T cells. As used herein, the terms “T-cell receptor” and “TCR” refer to a molecule found on the surface of the T cell responsible for recognizing the antigens that bind to MHC molecules, and customarily refer to a molecule capable of recognizing a peptide when presented by a MHC molecule. The molecule is a heterodimer including α and β chains (or selectively, γ and δ chains) or a TCR construct that generates signals. The TCR of the present invention is a hybrid TCR including the sequences derived from other species. For example, as mouse TCRs are more effectively expressed than human TCRs in human T cells, the TCR includes a human variable region and a murine constant region. The term also includes soluble TCR molecules, and derivatives thereof, as long as they include the complementarity determining regions (CDRs) as necessary for binding.

The XPRESIDENT® technology is described, amongst others, in WO 03/100432, WO 2005/076009, and WO 2011/128448, herewith incorporated by reference in their entireties.

In a preferred aspect of the above method, said developing improved personalized T-cell receptors, T-cells, and/or T-cell therapeutics against proliferative diseases further comprises transducing the patient's autologous (own) T-cells to express a tumor-specific exogenous T-cell receptor (TCR), and, optionally, suitably formulating said resulting T-cell therapeutics.

The present inventors demonstrate that the disulfide-modified HLA-A*02:01 molecule as an example can be readily generated as a stable and empty MHC monomer, loaded with ligand peptides after refolding, and used to generate affinity data in good agreement with data collected using wild type pMHC complexes.

Both disulfide-modified HLA-A*02:01 molecules and bispecific TCRs can be used jointly with BLI-based screenings to measure pMHC-bsTCR binding affinities, a platform with much higher throughput than surface plasmon resonance measurements presently used for these interactions in the literature. Disulfide-modified HLA-A*02:01 molecules are a piece of this platform, providing reliable yet high-throughput pMHC generation. This platform could also be useful for the analysis of other biologics if targeting pMHCs, like monoclonal antibodies or bispecifics (e.g. BITEs). The pMHC-bsTCR binding affinities correlated well with cellular assays when both were performed by the inventors with a functional bispecific T cell engager. To the inventors' knowledge, this is the first in depth analysis of the connection between pMHC-bsTCR binding affinity and the in vitro activity over a wide range of affinities. Compared to the cellular screenings, the affinity screening platform was easier to use and performed significantly quicker, therefore qualifying as an early screening tool. Due to the capability of the disulfide-modified HLA-A*02:01 molecules to predictably present even low affinity peptide ligands as pMHC complexes, the inventors can precisely measure pMHC-bsTCR binding affinities without having to account for variations encountered in exogenous peptide loading approaches, resulting in no loss of potentially valuable information. The inventors believe that the ease of use of the presented affinity analysis platform will aid the development of safe and effective T cell receptor based bispecific molecules from the early stages on.

As an example, the inventors show that it is possible to quickly generate pMHC-bsTCR binding affinity datasets and extrapolate cross-reactivity search motifs from them. Guided by the inventor's HLA peptidomics-based XPRESIDENT® platform, the search motifs can be used to identify potentially cross-reactive peptide ligands. In the presented execution of this strategy, the inventors were able to identify a large number of peptides strongly recognized by the bsTCR and capable of inducing T cell activation, with sequence consensus compared to the original target as low as one out of nine positions.

This exciting innovative technology could even lead to screenings of the entire discovered immunopeptidome: pMHC libraries of such dimensions are currently only available by yeast display using randomly mutated single-chain peptide MHC libraries (32, 33). While useful for broad TCR analysis, they are far more complicated in use and of less predictable peptide ligand composition compared to the peptide microarrays typically used in antibody development. Due to its stability and low-effort peptide loading process, the disulfide-modified HLA-A*02:01 molecules of the present invention may be the ideal fit for the creation of pMHC microarrays with high complexity in the future, for example by combining large scale coating of empty MHCs and the high-throughput of modern peptide microarray inkjet printers.

Major histocompatibility complex (MHC) class I molecules present short peptide ligands on the cell surface for interrogation by cytotoxic CD8+ T cells. MHC class I complexes presenting tumor-associated peptides (TUMAPs) are key targets of cancer immunotherapy approaches currently in development, making them important for efficacy as well as safety screenings. Without peptide ligand, MHC class I complexes are unstable and decay quickly, making the production of soluble monomers for analytical purposes labor intensive. The inventors have developed a disulfide bond stabilized HLA-A*02:01 molecules that are stable without peptide but can form peptide-MHC complexes with ligands of choice within minutes. The inventors illustrate the concurrence between the engineered mutants and the wild type variant with respect to the binding affinity of wild type or maturated high affinity TCRs. The inventors demonstrate their potential as analytes in high throughput affinity screenings of bispecific TCR molecules and generate a comprehensive TCR binding motif to identify off-target interactions.

Another aspect of the invention relates to nucleic acids encoding the stabilized MHC molecules or peptide binding fragments thereof of the second aspect of the invention and vectors. It is well known in the art that MHC I comprises all peptide binding domains, i.e. the alpha1 domain and alpha2 domain on one polypeptide chain whereas MHC II naturally comprises the alpha1 domain and the beta1 domain on two polypeptide chains. As previously noted a functional MHC II can also be provided on a single peptide by fusing the beta1 domain to the alpha1 domain. Accordingly, the nucleic acid encoding the MHC I and II of the invention may encode one or two polypeptides or the two polypeptides may also be encoded by two separate nucleic acids.

The term “nucleic acid” refers in the context of this invention to single or double-stranded oligo- or polymers of deoxyribonucleotide or ribonucleotide bases or both. Nucleotide monomers are composed of a nucleobase, a five-carbon sugar (such as but not limited to ribose or 2′-deoxyribose), and one to three phosphate groups. Typically, a nucleic acid is formed through phosphodiester bonds between the individual nucleotide monomers, In the context of the present invention, the term nucleic acid includes but is not limited to ribonucleic acid (RNA) and deoxyribonucleic acid (DNA) molecules but also includes synthetic forms of nucleic acids comprising other linkages (e.g., peptide nucleic acids as described in Nielsen et al. (Science 254:1497-1500, 1991). Typically, nucleic acids are single-or double-stranded molecules and are composed of naturally occurring nucleotides. The depiction of a single strand of a nucleic acid also defines (at least partially) the sequence of the complementary strand. The nucleic acid may be single or double stranded or may contain portions of both double and single stranded sequences. Exemplified, double-stranded nucleic acid molecules can have 3′ or 5′ overhangs and as such are not required or assumed to be completely double-stranded over their entire length. The nucleic acid may be obtained by biological, biochemical or chemical synthesis methods or any of the methods known in the art, including but not limited to methods of amplification, and reverse transcription of RNA. The term nucleic acid comprises chromosomes or chromosomal segments, vectors (e.g., expression vectors), expression cassettes, naked DNA or RNA polymer, primers, probes, cDNA, genomic DNA, recombinant DNA, CRNA, mRNA, tRNA, microRNA (miRNA) or small interfering RNA (SIRNA). A nucleic acid can be, e.g., single-stranded, double-stranded, or triple-stranded and is not limited to any particular length. Unless otherwise indicated, a particular nucleic acid sequence comprises or encodes complementary sequences, in addition to any sequence explicitly indicated.

Another aspect of the invention is a vector comprising the nuclei acid(s) encoding the stabilized MHC molecules or peptide binding fragments thereof of the second aspect of the invention. Such vectors may be used in vaccination strategies in which expression of the vaccine in the patient is desired. In such cases the vector may additionally encode the protein or T-cell epitope comprising fragments thereof, to which an immune response, preferably a T-cell response is desired. In this way it may be ascertained that the peptide binding pocket of the MHC molecule expressed in cells of the patient that comprise the vector is loaded with the correct peptide. Alternatively, the MHC molecule may be modified to comprise the peptide comprising the T-cell epitope in a fusion protein. Typically, the peptide will be fused to the MHC molecule with an intervening peptide linker to allow the peptide to be bound by the binding groove of the MCH molecule.

The term “vector” refers in the context of this invention to a polynucleotide that encodes a protein of interest or a mixture comprising polypeptide(s) and a polynucleotide that encodes a protein of interest, which is capable of being introduced or of introducing proteins and/or nucleic acids comprised therein into a cell. Examples of vectors include but are not limited to plasmids, cosmids, phages, viruses or artificial chromosomes. A vector is used to introduce a gene product of interest, such as e.g. foreign or heterologous DNA into a host cell. Vectors may contain “replicon” polynucleotide sequences that facilitate the autonomous replication of the vector in a host cell. Foreign DNA is defined as heterologous DNA, which is DNA not naturally found in the host cell, which, for example, replicates the vector molecule, encodes a selectable or screenable marker, or encodes a transgene. Once in the host cell, the vector can replicate independently of or coincidental with the host chromosomal DNA, and several copies of the vector and its inserted DNA can be generated. In addition, the vector can also contain the necessary elements that permit transcription of the inserted DNA into an mRNA molecule or otherwise cause replication of the inserted DNA into multiple copies of RNA. Vectors may further encompass “expression control sequences” that regulate the expression of the gene of interest. Typically, expression control sequences are polypeptides or polynucleotides such as promoters, enhancers, silencers, insulators, or repressors. In a vector comprising more than one polynucleotide encoding for one or more gene products of interest, the expression may be controlled together or separately by one or more expression control sequences. More specifically, each polynucleotide comprised on the vector may be control by a separate expression control sequence or all polynucleotides comprised on the vector may be controlled by a single expression control sequence. Polynucleotides comprised on a single vector controlled by a single expression control sequence may form an open reading frame. Some expression vectors additionally contain sequence elements adjacent to the inserted DNA that increase the half-life of the expressed mRNA and/or allow translation of the mRNA into a protein molecule. Many molecules of mRNA and polypeptide encoded by the inserted DNA can thus be rapidly synthesized. Such vectors may comprise regulatory elements, such as a promoter, enhancer, terminator and the like, to cause or direct expression of said polypeptide upon administration to a subject. Examples of promoters and enhancers used in the expression vector for animal cell include early promoter and enhancer of SV40 (Mizukami T. et al. 1987), LTR promoter and enhancer of Moloney mouse leukemia virus (Kuwana Y et al. 1987), promoter (Mason JO et al. 1985) and enhancer (Gillies SD et al. 1983) of immunoglobulin H chain and the like. Any expression vector for animal cell can be used, as long as a gene encoding the human antibody C region can be inserted and expressed. Examples of suitable vectors include pAGE107 (Miyaji H et al. 1990), pAGE103 (Mizukami T et al. 1987), pHSG274 (Brady G et al. 1984), pKCR (O'Hare K et al. 1981), pSG1 beta d2-4-(Miyaji H et al. 1990) and the like. Other examples of plasmids include replicating plasmids comprising an origin of replication, or integrative plasmids, e.g. pUC, pcDNA, pBR.

In summary, the invention relates to the following items.
Item 1. A method for screening for a TCR-binding peptide ligand/MHC molecule complex, comprising the steps of: a) providing a suitably stabilized MHC molecule, wherein said MHC molecule comprises at least one artificially introduced covalent bridge between amino acids of the alpha1 domain and amino acids of the alpha2 domain of said stabilized MHC molecule in case of MHC I, and at least one artificially introduced covalent bridge between amino acids of the alpha1 domain and amino acids of the beta1 domain of said stabilized MHC molecule in case of MHC II, b) contacting said suitably stabilized MHC molecule with a multitude of peptide ligands thereof, to form peptide ligand/MHC (pMHC) molecule complexes, and c) screening said pMHC molecule complexes for TCR-binding.
Item 2. The method according to Item 1, wherein said MHC molecule is HLA, or a multimer of HLA, MHC I or MHC II, selected from the group consisting of a dimer, a trimer and a tetramer.
Item 3. The method according to Item 1 or 2, wherein said at least one artificially introduced covalent bridge between amino acids is selected from a recombinantly introduced disulfide bridge, the introduction of non-natural amino acids to be crosslinked, the introduction of photo-crosslinking amino acids, and chemically introduced crosslinks.
Item 4. The method according to any one of Items 1 to 3, wherein said at least one artificially introduced covalent bridge between amino acids is introduced between α-helices, for example by mutating a tyrosine at position 84 and an alanine at position 139 into cysteines of MHC I.
Item 5. The method according to any one of Items 1 to 4, wherein said multitude of peptide ligands comprises at least about 1,500 different MHC binding peptides, preferably at least about 5,000 different MHC binding peptides, more preferred at least about 15,000 different MHC binding peptides, and most preferred an immunopeptidome preparation with at least about 150,000 MHC binding peptides.
Item 6. The method according to any one of Items 1 to 5, wherein said contacting comprises loading said MHC binding peptides at between about 4° C. to 30° C., preferably at about room temperature.
Item 7. The method according to any one of Items 1 to 6, wherein said loaded HLA/peptide molecules are stable for more than about 1 day, and preferably for more than 1 week at about 4° C.
Item 8. The method according to any one of Items 1 to 7, wherein the sensitivity level for affinity screening of a TCR for binding to pMHC complexes is higher than about K_{d} 1.0×10⁻⁹, preferably higher than about K_{d} 1.0×10⁻⁶ M, and more preferred higher than about K_{d} 1.0×10⁻³ M.
Item 9. The method according to any one of items 1 to 8, wherein said TCR is selected from a native TCR, a soluble TCR molecule, and a TCR-like molecule, such as a bs TCR.
Item 10. The method according to any one of items 1 to 9, wherein either the TCR or the MHC molecule is suitably immobilized on a solid surface, such as a chip, biosensor, glass slide or bead.
Item 11. The method according to any one of items 1 to 10, wherein said TCR and/or the MHC molecule is/are label and/or marker-free.
Item 12. The method according to any one of items 1 to 11, wherein said method is performed as a high-throughput screening format.
Item 13. A method for detecting or generating a specific amino acid binding motif for a TCR, comprising performing the method according to any one of items 1 to 12 comprising a preselected TCR, and the additional step of determining and comparing the amino acid sequences of those peptide ligands in said peptide ligand/MHC molecule complexes for which a TCR binding was detected, thereby identifying the specific amino acid binding motif for said preselected TCR.
Item 14. The method according to item 13, wherein said peptide ligand/MHC molecule complexes are used in parallel assay reactions having different concentrations.
Item 15. The method according to item 13 or 14, wherein said method steps are repeated comprising a pool of peptides consisting of modified amino acid binding motifs for said preselected TCR as identified.
Item 16. A method for detecting or determining cross-reactivity of a TCR, comprising performing the method according to item 15, and the additional step of determining and comparing the amino acid sequences of those peptide ligands in said peptide ligand/MHC molecule complexes for which a TCR binding was detected, thereby identifying cross-reactivity of said TCR.
Item 17. A method for detecting or determining cross-reactivity of a TCR, comprising performing the method according to any one of items 1 to 12 comprising a preselected TCR, and the additional step of determining and comparing the amino acid sequences of those peptide ligands in said peptide ligand/MHC molecule complexes for which a TCR binding was detected, thereby identifying cross-reactivity of said TCR.
Item 18. The method according to any one of items 1 to 17, further comprising the step of measuring T cell activation comprising a TCR and a TCR-binding peptide ligand/MHC molecule complex that binds said TCR.
Item 19. A method for activating and/or stimulating and/or expanding a cell population (e.g. specific T cell population) with a peptide ligand/MHC molecule complex carrying stimulation framework, where said framework compromises a peptide ligand/MHC molecule complex immobilized on a carrier, e.g. beads, filaments, nanoparticles, or any carrier capable of carrying said complex, where a suitably stabilized MHC complex can be immobilized onto the carrier and the framework stored in such a state for a prolonged time prior to addition of the peptide ligand, thus significantly increasing the practicability of such a stimulation framework mimicking antigen presenting cells in research or clinical practices.
Item 20. A pharmaceutical composition comprising a suitably stabilized MHC molecule, wherein said MHC molecule comprises at least one artificially introduced covalent bridge between amino acids of the alpha1 domain and amino acids of the alpha2 domain of said stabilized MHC molecule in case of MHC I, and at least one artificially introduced covalent bridge between amino acids of the alpha1 domain and amino acids of the beta1 domain of said stabilized MHC molecule in case of MHC II, wherein said stabilized MHC molecule is bound to a bead, filament, nanoparticle or other suitable carrier.
Item 21. The pharmaceutical composition according to item 20, further comprising one or a combination of more costimulatory molecules and/or a chronological sequence of these costimulatory molecules, such as, for example, an anti CD28 antibody or anti 41BB antibody.
Item 22. The pharmaceutical composition according to item 20 or 21, wherein said stabilized MHC molecule can be stored for a prolonged time prior to addition of the peptide ligand, e.g. at room temperature or 4° C. or about −80° C.
Item 23. Use of the pharmaceutical composition according to any one of items 20 to 22 in a method according to any of items 1 to 19.

The present invention will now be further described in the examples with reference to the accompanying figures, nevertheless, without wanting to be limited thereto. For the purposes of the present invention, all references as cited are incorporated by reference in their entireties.

### BRIEF DESCRIPTION OF THE DRAWINGS

FIG. 1 shows an overview of disulfide-stabilized HLA-A*02:01 production and use for affinity measurements. (a) Expression plasmids of heavy chain and β₂m are transfected into E. coli and proteins of interest expressed in inclusion bodies. HLA monomers are purified using size exclusion. (b) Empty disulfide modified HLA-A*02:01 molecules can be loaded with peptide ligands by incubation at room temperature. For affinity measurements, they are immobilized onto functionalized biosensors, e.g. by biotin streptavidin interaction, and used to record association and dissociation of TCRs or TCR-like molecules.

FIGS. 2A-2D show the association and dissociation behavior of 1G4 TCR with different MHC monomers. Raw data is shown in FIGS. 2A and 2B, curve fittings in FIG. 2C and 2D. All measurements performed as 1:2 analyte dilution series starting at 24 μM. (FIG. 2A) Binding curve of the 1G4 TCR against immobilized ESO 9V Y84C/A139C HLA-A*02:01 pMHC. (FIG. 2B) Binding curve of the 1G4 TCR against immobilized ESO 9V WT-A*02:01 pMHC. (FIG. 2C) Binding curve of the 1G4 TCR against immobilized empty Y84C/A139C HLA-A*02:01. (FIG. 2D) Binding curve of the 1G4 TCR against immobilized SL9 Y84C/A139C HLA-A*02:01 pMHC.

FIGS. 3A-3D show the affinities of the SL9 specific bs-868Z11-CD3 bsTCR with different MHC monomers and peptide ligands. (FIG. 3A) Binding curve of bs-868Z11-CD3 against immobilized SL9 Y84C/A139C HLA-A*02:01 pMHC. Raw data is shown in FIGS. 3A and 3B, curve fittings in FIGS. 3C and 3D. Measured using 1:2 analyte dilution series starting at 500 nM. (FIG. 3B) Binding curve of bs-868Z11-CD3 against immobilized SL9 WT-A*02:01 pMHC. Raw data is displayed in black, curve fittings in red. Measured using 1:2 analyte dilution series starting at 500 nM. (FIG. 3C) Binding curve of bs-868Z11-CD3 against immobilized empty Y84C/A139C HLA-A*02:01. Measured using 1:2 analyte dilution series starting at 500 nM. (FIG. 3D) Correlation between affinities measured using Y84C/A139C HLA-A*02:01 pMHCs or WT-A*02:01 pMHC complexes generated using UV-exchange. K_{d}s were plotted for 140 different peptide ligands generated using both methods and measured during successive experiments with good curve fittings. K_{d}s were fitted using 500 nM and 158 nM analyte concentrations. R² is the calculated correlation coefficient, dashed line represents optimal ratio.

FIGS. 4A-4C show the binding motif of bs-868Z11-CD3 generated using Y84C/A139C HLA-A*02:01 generated mutated amino acid sequence library as soluble analyte and immobilized bsTCR. K_{d}s were fitted using curves from at least one and up of the inventors' analyte concentrations with at least a peak signal of 0.05 nm for curves to be included. Positions with no fittable curves were assigned a K_{d} of 5×10⁻⁶ M. Measured using 1/\sqrt{10} analyte dilution series starting at 500 nM. (FIG. 4A) Heat map of affinities depending on the amino acid introduced and the exchanged position in the peptide sequence. White squares indicate wild type peptide amino acid. (FIG. 4B) Visualization of the binding motif as seq2logo graph. Size of individual letters inversely represents measured affinity for the respective amino acid at this position, calculated using the inverse K_{d} value divided by 10⁸ and the PSSM-Logo algorithm. (FIG. 4C) Binding curve of bs-868Z11-CD3 bsTCR against ALYNVLAKV (SEQ ID NO: 1) loaded Y84C/A139C HLA-A*02:01 pMHC. Measured using 1/\sqrt{10} analyte dilution series starting at 500 nM.

FIGS. 5A-5E show the result of coincubation assays with peptide loaded target cells, Jurkat effector cells and bs-868Z11-CD3 at six different concentrations. (FIG. 5A) Measured fold-induction above background for Jurkat cells stimulated at different concentrations of bs-868Z11-CD3 in presence of SL9 wild type peptide loaded T2 target cells. (FIG. 5B) Correlation of measured affinity for the peptide ligands from the positional scanning library with the lowest bsTCR concentration necessary to induce 3-fold luminescence increase over background. Peptides are grouped into 9 different groups depending on the location of the exchange in the wild type sequence. (FIG. 5C) Correlation of measured affinity for the peptide ligands from the positional scanning library with their NetMHC predicted pMHC binding rank. Peptides are grouped into 6 different groups depending on the lowest bsTCR concentration necessary to induce 3-fold luminescence increase over background. (FIG. 5D) Correlation of measured affinities for the cross-reactive peptide ligand candidates with the lowest bsTCR concentration necessary to induce 3-fold luminescence increase over background. (FIG. 5E) Measured fold-induction above background for Jurkat cells stimulated at different concentrations of bs-868Z11-CD3 in presence of ALYNVLAKV (SEQ ID NO: 1) peptide loaded T2 target cells. Error bars represent biological triplicates.

FIGS. 6A and 6B show the comparison of Y84C/A139C HLA-A*02:01 or UV exchange generated WT-A*02:01 pMHC complexes as soluble analytes for affinity measurements with immobilized bs-868Z11-CD3. Y84C/A139C HLA-A*02: 01 complexes left, WT-A*02:01 complexes right. All measurements were performed using 1:2 analyte dilution series starting at 500 nM.

FIG. 7 shows the crystal structure of ESO 9VY84C/A139C HLA-A*02:01 and ESO 9V WT-A*02:01 in complex with 1G4. (a) Overlay of WT and Y84C/A139C HLA-A*02:01 structure with a focus on peptide and amino acid side-chain orientation. (b) Close-up of the F-pocket and the introduced disulfide bond between α1 and α2. (c) Overlay of the 1G4 CDR loops interacting with the peptide and the MHC backbone. (d) Overlay of both crystal structures from a lateral perspective. Error bars represent biological triplicates.

FIG. 8 shows the binding motif of bs-868Z11-CD3 generated using Y84C/A139C HLA-A*02:01 generated positional scanning library as soluble analyte and immobilized bsTCR. Measurements were performed using four soluble analyte concentrations. Positions with no fittable curves were assigned a K_{d} of 5×10⁻⁶ M. Soluble analyte concentration range produced by 1/analyte dilution series starting at 500 nM. Heat map of affinities depending on the amino acid introduced and the exchanged position in the peptide sequence.

FIG. 9 shows an illustration of bsTCR bs-868Z11-CD3 construct. The 868Z11 domain is based on the SLYNTVATL-reactive TCR 868 and incorporates affinity enhancing mutations in the CDR2β (YYEEEE to YVRGEE) and CDR3α region (CAVRTNSGYALN to CAVRGAHDYALN) identified by Varela-Rohena et al. (8). The Vβ and Vα domains of the affinity enhanced TCR were linked through a single chain linker (GSADDAKKDAAKKDGKS) and further modified with a surface stability conferring mutation in the Vα2 region (F49S) to allow for soluble expression by Aggen et al.(22). To create the bs-868Z11-CD3 molecule, this 868Z11 scTv domain was fused to the F(ab′) heavy chain portion of a humanized anti-CD3 antibody through an IgG2 derived CH2 hinge domain (APPVAG) with two cysteine-knock-outs (C₂₂₆S and C₂₂₉S), incorporated to prevent the formation of F(ab′)₂ homodimers on expression.

FIGS. 10A and 10B show an analysis of UV exchange efficiency and Octet measurement results for 28 different peptides selected from SLYNTVATL based positional scanning library. (FIG. 10A) Left axis: pMHC concentration after UV exchange with 25000 ng/ml of UV-sensitive pMHC monomer determined using an anti-β2m ELISA. Dotted line represents ELISA/UV exchange background signal based on an UV exchange without peptide. Error bars represent technical triplicates. Right axis: Ratio of binding responses of soluble pMHC analytes to immobilized bs-868Z11-CD3 on Octet RED384 system. pMHCs were either prepared using UV exchange or by Y84C/A139C HLA-A*02:01 peptide loading. Ratios calculated by dividing UV-A*02:01 response by the Y84C/A139C HLA-A*02:01 response after 60 s of association with similarly loaded anti-F(ab) biosensors. (FIG. 10B) Detailed curve fittings for four peptides with NetMHC ranks 15 and larger. Y84C/A139C HLA-A*02:01 complexes left, WT-A*02:01 complexes right. All measurements were performed using 1:2 analyte dilution series starting at 500 nM.

FIG. 11 shows binding of multiple different soluble TCRs and bsTCR bs-868Z11-CD3 to non-loaded Y84C/A139C HLA-A*02:01 or Y84C/A139C HLA-A*02:01 loaded with an irrelevant peptide. (a) Binding of three different HLA-A*02:01 restricted soluble TCRs as well as bs-868Z11-CD3 to functionally-empty Y84C/A139C HLA-A*02:01. Y84C/A139C HLA-A*02:01 was immobilized onto a streptavidin sensor, each TCR supplied at 1 mg/ml (20 μM for soluble TCRs, 13.3 μM for bsTCR). (b) Binding of the same TCRs to Y84C/A139C HLA-A*02:01 loaded with an irrelevant peptide.

FIG. 12 shows octet affinity measurements for Y84C/A139C HLA-A*02:01 SLYNTVATL pMHC with immobilized bs-868Z11-CD3 directly after exchange and after 2 weeks of storage at 4° C. Both measurements were performed using 1:2 analyte dilution series starting at 277.8 nM.

FIGS. 13A and 13B show a multiple sequence alignment of various HLA alleles and one murine allele. In the sequence alignment the areas for introducing stabilizing amino acids substitutions are highlighted. This alignment provides the skilled person with a basis to determine in each given HLA allele the amino acids to be substituted in order to stabilize the MHC molecule.

FIG. 14 shows the affinities of the SL9 specific bs-868Z11-CD3 bsTCR towards SL9 pMHC produced with different disulfide-modified HLA-A*02:01 complexes. Binding curves show bs-868Z11-CD3 association and dissociation against immobilized SL9 pMHCs. Measured using 1:2 analyte dilution series starting at 500 nM. Binding curve of the bs-868Z11-CD3 bsTCR against immobilized SL9 WT-HLA*02:01 pMHC (upper left graphic). Binding curve of the bs-868Z11-CD3 bsTCR against immobilized SL9 Y84C/A139C HLA*02:01 pMHC (upper right graphic). Binding curve of the bs-868Z11-CD3 bsTCR against immobilized SL9 F22C/S71C HLA*02:01 (lower left graphic). Binding curve of the bs-868Z11-CD3 bsTCR against immobilized SL9 F22C/S71C W51C/G175C HLA-A*02:01 pMHC (lower right graphic).

FIG. 15 shows K_{d} values of a high affinity TCR to different pMHC complexes. In each case the K_{d} of the WT-A*02:01 molecule is shown on the X-axis and the K_{d} of the two different disulfide-modified HLA-A*02:01 MHC molecules is shown on the y-axis and each dot represents one of different peptides loaded in the MHC molecule.

SEQ ID NOs 1 to 5 and 16 to 325 show peptide sequences as used in the examples, below.

### EXAMPLES

### 1. Peptide Synthesis

All peptides were generated in house using standard Fmoc chemistry with a Syro II peptide synthesizer. Peptides were subsequently analyzed using HPLC and had an average purity of 74%. UV-light sensitive peptides contained a light-sensitive building block with a 2-nitrophenylamino acid residue. The dipeptide GM was procured from Bachem. Before use peptides were solved in DMSO (Sigma, Cat. Nr. 41640), 0.5% TFA (Sigma, Cat. Nr. T6508) at concentrations ranging from 2 mg/ml to 10 mg/ml depending on the desired use case.

### 2. Generation of MHC Complexes by Refolding and Purification

Recombinant HLA-A*02:01 wild type (WT-A*02:01, SEQ ID NO: 322) or disulfide modified HLA-A*02:01 heavy chains with C-terminal BirA signal sequences and human β₂m light chain were produced in Escherichia coli as inclusion bodies and purified as previously described (2). HLA-A*02:01 complex refolding reactions were performed as previously described with minor modifications (Saini et al 2013). In brief, WT-A*02:01 or disulfide-modified HLA-A*02:01 heavy chains, β₂m light chain and peptide were diluted in refolding buffer (100 mM Tris·Cl pH 8, 0.5 M arginine, 2 mM EDTA, 0.5 mM oxidized glutathione, 5 mM reduced glutathione) and incubated for 2 to 8 days at 4° C. while stirring before concentration. The concentrated protein was purified by size exclusion chromatography (SEC) in 20 mM Tris-HCl, pH 8/150 mM NaCl on an ÄKTAprime system (GE Healthcare) using a HiLoad 26/600 200 pg column (GE Healthcare). Peak fraction was either concentrated directly to 2000 μg/ml, aliquoted and frozen at −80° C. or biotinylated by BirA biotin-protein ligase (Avidity) overnight at 4° C. according to the manufacturer's instructions and subjected to a second gel-filtration before final concentration to 2000 μg/ml, aliquotation and storage at −80° C.

To produce HLA-A*02:01 wild type peptide-MHC complexes 9mer (full length) peptides or UV-light sensitive 9mer peptides (full length) were added to the refolding buffer at a concentration of 30 μM. To produce empty Y84C/A139C HLA-A*02:01 (SEQ ID NO: 323) complexes the dipeptide GM was added to the refolding buffer at a concentration of 10 mM. To produce F22C/S71C HLA-A*02:01 (SEQ ID NO: 324) complexes no peptide was added to the refolding buffer. To produce F22C/S71C W51C/G175C HLA-A*02:01 (SEQ ID NO: 325) complexes no peptide was added to the refolding buffer.

Table 1 below shows the refolding methods of the different disulfide-modified HLA-A*02:01 molecules and the WT-A*02:01 molecule:

TABLE 1

| | Refolding Method | | |
|---|---|---|---|
| | Full Length | | Without |
| | Peptide | Dipeptide | Peptide |
| HLA-A*02:01 wild | + | − | − |
| type (SEQ ID | | | |
| NO: 322) | | | |
| HLA-A*02:01 | + | + | − |
| 84/139 (SEQ ID | | | |
| NO: 323) | | | |
| HLA-A*02:01 22/71 | n.d. | n.d. | + |
| (SEQ ID NO: 324) | | | |
| HLA-A*02:01 22/71 | n.d. | n.d. | + |
| 51/175 (SEQ ID | | | |
| NO: 325) | | | |

+: Protein is refoldable;
− protein is not refoldable.

3. Generation of Peptide Exchanged HLA-A*02:01 pMHC Complexes Using UV Mediated Peptide Ligand Exchange or Empty Disulfide-Modified HLA-A*02: 01 Molecules

Peptide exchange reactions with UV-light cleavable peptides were performed as previously described. In short desired nonamer peptides were mixed with biotinylated UV light-sensitive pMHC complexes at 100 to 1 molar ratio and subjected to at least 30 minutes of 366 nm UV light (Camag).

Peptide loading reactions with empty disulfide-modified HLA-A*02:01 MHC complexes were performed by addition and mixing of desired peptides of at least a 100 to 1 molar ratio to the monomer solution and 5-minute incubation at room temperature.

### 4. Soluble TCR Production

Soluble TCRs were produced as previously described (20). In short TCR alpha and TCR beta chain constructs were expressed separately in Escherichia coli as inclusion bodies and purified. TCR alpha chains are mutated at position 48 by replacing a threonine with a cysteine and TCR beta chains at position 57 by replacing a serine with a cysteine to form an inter-chain disulfide bond.

5. bsTCR Design and Production

The bs-868Z11-CD3 molecule was generated by linking the scTv 868Z11 to the C-terminus of the F(ab′)-domain of a humanized antiCD3-antibody (22, 23). To this end the V_{β}-domain of the scTv was directly fused to the upper CH2-region derived from human IgG2 (APPVAG, SEQ ID NO: 2). Cysteine-knock-outs C₂₂₆S and C₂₂₉S within the hinge prevent the formation of F(ab)₂ molecules. HCMV-driven expression vectors coding either for the construct described above or the light chain of the humanized antiCD3-antibody were transiently co-transfected in ExpiCHO cells (Thermo). After 12 days supernatant was processed by tandem chromatography (protein L followed by preparative size exclusion, GE Biosciences) and highly pure monomeric bsTCR was formulated in PBS

### 6. OctetRED Based Bio-Layer Interferometry Kinetic Affinity Measurements

The affinity of sTCR or bsTCR molecules for different pMHC complexes was measured on an OctetRED 384 system (Pall Fortebio) using kinetic or steady state binding analysis. All analytes or ligands were diluted to their final concentration in kinetics buffer (PBS, 0.1% BSA, 0.05% TWEEN 20) if not specified otherwise. All biosensors were hydrated for at least 10 minutes in kinetics buffer before use. Loadings and measurements were performed in 384 tilted well plates (Pall Fortébio) with at least 40 μl at a 3 mm sensor offset. Plate temperature was set at 25° C. and shaker speed at 1000 rpm. To allow inter-step correction baselines before association phases and the following dissociation phase were performed in the same well. Kinetics buffer was used as dissociation buffer with DMSO at an appropriate concentration added if necessary to match the analyte composition.

In the case of pMHC immobilization dip and read streptavidin (SA; Pall Fortébio Cat. Nr. 18-5021) biosensors were used to immobilize biotinylated pMHC monomers at a presumed concentration of 25 μg/ml for 60 seconds followed by a 60 seconds baseline and association and dissociation phases of 60 seconds each if not specified otherwise.

In the case of bsTCR immobilization dip and read anti-human Fab-CH1 2^{nd} generation (FAB2G; Pall Fortébio Cat. Nr. 18-5127) biosensors were used to immobilize bsTCR molecules at a concentration of 100 μg/ml for 60 seconds, followed by a 15 seconds baseline and association and dissociation phases of 60 seconds each if not specified otherwise. FAB2G biosensor were regenerated up to 4 times by incubating the loaded biosensor for 5 seconds each in 10 mM Glycine pH1.5 and kinetics buffer consecutively for three times. FAB2G were also pre-conditioned that way before their first ligand immobilization.

All sensorgrams were analyzed using the OctetRED software “Data Analysis HT” version 10.0.3.7 (Pall Fortébio). Raw sensor data was aligned at the Y axis by aligning the data to the end of the baseline step and inter-step correction was used to align the start of the dissociation to the end of the association phase. No Savitzky-Golay filtering was applied. Resulting sensorgrams were then fitted using a 1:1 Langmuir kinetics binding model.

### 7. Cell Lines

The TAP-deficient HLA-A*02:01 expressing cell line T2 was procured from ATCC (CRL-1992) and cultured in RPMI Medium 1640 GlutaMAX™ (Thermo Fisher, Cat. Nr. 61870010) supplemented with 10% heat inactivated FCS (Life Technologies, Cat. Nr. 10270106) and the antibiotics penicillin and streptomycin (Biozym, Cat. Nr. 882082, 100 μg ml⁻¹ each) up until passage number 16 if necessary. The GloResponse™ NFAT-luc2 Jurkat cell line was procured from Promega (Cat. Nr. CS1764) at passage number 6 and cultured in RPMI Medium 1640 GlutaMAX™ (Thermo Fisher, Cat. Nr. 61870010) supplemented with 10% heat inactivated FCS (Life Technologies, Cat. Nr. 10270106), 1% Sodium Pyruvate (C.C.Pro, Cat. Nr. Z-20M) and the antibiotics hygromycin B (Merck Millipore, Cat. Nr. 400052, 200 μg/ml), penicillin and streptomycin (Biozym, Cat. Nr. 882082, 100 μg/ml each) up until passage number 14, if necessary.

### 8. T Cell Activation Assay

T cell activation assays using GloResponse™ NFAT-luc2 Jurkat cells and peptide loaded T2 target cells were performed according to manufacturer instructions. In short, T2 cells were harvested from continuous cell culture, washed and resuspended in T2 culture medium at a concentration of 3.3×10⁶ cells/ml and transferred to 96 well round bottom plates (Corning costar®, Cat. Nr. 3799). Peptide in DMSO, 0.5% TFA was added to a final concentration of 100 nM and the suspension incubated for 2 to 3 hours at 37° C., 5% CO₂. bsTCR formulated in PBS was diluted in T2 culture medium to desired concentration and 25 μl of the respective dilution was distributed to white 96 well flat bottom plates (Brand, Cat. Nr. 781965). GloResponse™ NFAT-luc2 Jurkat cells were harvested from continuous cell culture, washed and resuspended in T2 culture medium at a concentration of 3.0×10⁶ cells ml⁻¹ and 25 μl of the cell suspension was distributed to the white 96 well flat bottom plates with bsTCR dilutions.

After peptide loading T2 cells were resuspended and 25 μl distributed to the white 96 well flat bottom plates with bsTCR dilutions and GloResponse™ NFAT-luc2 Jurkat cells for a final effector to target ratio of 1:1 (75.000 cells each). Fully assembled plates were mixed for 5 minutes at 300 rpm on a plate shaker and the incubated for 18 to 20 h at 37° C., 5% CO₂. After the incubation period 75 μl of Bio-Glo™ luciferase reagent was added to each well and the plates incubated for minutes at 300 rpm on a plate shaker in the dark before reading luminescence at a 0.5 second integration time with a Synergy2 plate reader (Biotek). Luminescence as measured in relative light units (RLU) was converted to fold induction for each well by dividing measured RLU through those of control wells.

### 9. Crystallization and Imaging

The Y84C/A139C HLA-A*02:01-SLLMWITQV complex and the 1G4 TCR were concentrated and mixed in a 1:1 ratio to achieve a concentration of 7 mg/ml for crystallization. A sitting drop vapor diffusion experiment resulted in crystals in the presence of a mother liquor containing 0.1 M ammonium acetate, 0.1 M bis-tris (pH 5.5), and 17% polyethylene glycol (PEG) 10,000. A single crystal was transferred to a cryoprotectant solution containing 0.1 M ammonium acetate, 0.1 M bis-tris (pH 5.5), 20% (w/v) PEG 10,000, and 10% glycerol. The crystal was mounted and cryocooled at 100 K on the EMBL P14 beamline at Deutsche Elektronen-Synchrotron containing an EIGER 16M detector. An x-ray dataset was collected to a resolution of 2.5 Å (Table 2).

TABLE 2
Data collection and refinement statistics 1G4/
Y84C/A139C HLA-A*02:01/SLLMWITQV

| | 1G4/Y84C/A139C |
|---|---|
| | HLA-A*02:01/SLLMWITQV |
| Data collection | |
| Space group | P2₁ |
| Cell dimensions [symbol] | |
| a, b, c (Å) | 75.44, 53.67, 121.74 |
| [symbol] (°) | 90.0 98.0 90.0 |
| Resolution (Å) | 2.50 (2.60-2.50)* |
| Rₚᵢₘ | 0.037 (0.69) |
| // [symbol] / | 9.6 (1.1) |
| CC (1/2) | 100.0 (0.68) |
| Completeness (%) | 99.3 (99.1) |
| Redundancy | 4.8 (5.0) |
| Refinement | |
| Resolution (Å) | 30-2.50 |
| No. reflections | 33552 |
| R_{work}/R_{free} | 0.229 (0.273) |
| No. atoms | |
| Protein | 3180 |
| Ligand/ion | 19 |
| Water | 589 |
| B-factors | |
| Protein | 98.1 |
| Ligand/ion | 97.8 |
| Water | 66.2 |
| R.m.s. deviations | |
| Bond lengths (Å) | 0.002 |
| Bond angles (°) | 0.47 |

The data were processed with XDS and scaled with AIMLESS (35, 36). Molecular replacement was performed using MOLREP with the coordinates of the TCR portion of the native complex first, followed by the pMHC [Protein Data Bank (PDB) 2BNR], and the structure was refined with REFMAC5 (37, 38). The engineered disulfide bond was manually built with Coot (39). The structure was refined to an R factor of 22.9% (R_{free} of 27.3%). MolProbity was used to validate the geometry and indicated that 93.9% of the residues were in the allowed regions of the Ramachandran plot [with one glycine residue (Gly143) in the disallowed regions] (40).

### 10. Motif-Based Identification of Potentially Cross-Reactive Peptide Ligands

Searches for nonamer peptide ligands matching one of the potential combinations allowed by the search motif were performed using the NCBI human protein database. This database covers all nonredundant GenBank CDS translations, as well as records from PDB, SwissProt, PIR, and PRF but excluding environmental samples from the whole-genome shotgun projects. The database was directly acquired from the NCBI servers.

### 11. Seq2Logo Generation

Seq2Logos visualizing the binding motif were created by taking the inverse value of measured K_{d} values for the respective interaction and dividing them by 10⁸. These values were assembled in the form of a position-specific scoring matrix file and processed using the PSSM-Logo type at the Seq2Logo online resource of the Denmark Technical University Bioinformatics department (27).

### 12. Peptide Binding Measured By Fluorescence Anisotropy

Peptide binding was evaluated in fluorescence anisotropy assay with 300 nM of purified refolded Y84C/A139C HLA-A*02:01. 100 nM of the fluorescently labeled high-affinity peptide NLVPK_{FITC}VATV (Genecast) was added to the folded Y84C/A139C HLA-A*02:01 and kinetic measurements were performed with Tecan Infinite M1000 PRO (Tecan, Crailsheim, Germany) multimode plate reader measuring anisotropy (FITC λₑₓ=494 nm, λₑₘ=517 nm). Y84C/A139C HLA-A*02:01 were either used directly after refolding or preserved at −80° C. in storage buffer (10% Glycerol, 50 mM Tris-HCL, pH 8.0) for the indicated amount of time before measurement. The kinetic measurements were performed at room temperature (22-24° C.) in 50 mM HEPES buffer, pH 7.5. Data was plotted using GraphPad Prism v7.

### 13. Anti-beta-2 Microglobulin ELISA

Streptavidin (Molecular Probes, Cat. Nr. S888) at a final concentration of 2 μg/ml in PBS was added to Nunc MAXIsorp plates (Thermo Fisher, Cat. Nr. 439454) and sealed plates incubated over night at room temperature in a damp environment. The following day plates were washed 4 times with washing buffer (PBS, 0.05% TWEEN-20) using a ELx405 plate washer (Biotek). 300 μl blocking buffer (PBS with 2% BSA) was added to each well and sealed plates incubated at 37° C. for 1 hour. Blocking buffer was discarded before adding 100 μl of a 1:100 dilution in blocking buffer of the respective UV exchange pMHC preparation. A standard series ranging from 500 ng/ml to 15.6 ng/ml based on a conventionally refolded pMHC monomer was included on each plate. Edge wells were filled with 300 μl blocking buffer to reduce edge effects and sealed plates were incubated at 37° C. for 1 hour. Plates were again washed 4 times before adding 100 μl anti-beta 2 microglobulin HRP conjugated secondary antibody (Acris, Cat. Nr. R1065HRP) at a final concentration of 1 μg/ml to each well. Sealed plates were incubated at 37° C. for 1 hour. Plates were washed again 4 times with washing buffer before adding 100 μl of room temperature TMB substrate (Sigma, Cat. Nr. T0440) to each well. Plates were incubated for 5 minutes at room temperature before stopping by adding 50 μl 1N H₂SO₄ to each well. Plates were immediately analyzed by reading absorbance at 450 nm for 5 seconds using a Synergy2 plate reader. pMHC concentration was calculated based on standard curve fitting (Log(Y)=A*Log(X)+B) using the Synergy2 software. Data was plotted using GraphPad Prism v7.

### 14. Flow Cytometric T2 Peptide Binding Assay

The TAP-deficient HLA-A*02:01-expressing cell line T2 was procured from ATCC (CRL-1992) and cultured in RPMI Medium 1640 GlutaMAX™ (Thermo Fisher, Cat. Nr. 61870010) supplemented with 10% heat inactivated FCS (Life Technologies, Cat. Nr. 10270106) and the antibiotics penicillin and streptomycin (Biozym, Cat. Nr. 882082, 100 μg/ml each) up until passage number 16 if necessary. T2 cells were harvested from continuous cell culture, washed and resuspended in T2 culture medium at a concentration of 3.3×10⁶ cells/ml and transferred to 96 well round bottom plates (Corning costar®, Cat. Nr. 3799). Peptide in DMSO, 0.5% TFA was added to a final concentration of 10 μM and the suspension incubated for 2 hours 37° C., 5% CO₂. Plates were washed twice with PFEA (PBS, 2% FCS, 2 mM EDTA, 0.01% sodium azide) before addition of 50 μl PE labelled anti-human HLA-A2 (Biolegend, Cat. Nr. 343305) per well diluted 1:250 with PFEA to a final concentration of 0.8 μg/ml. Plates were incubated at 4° C. for 30 minutes before being washed twice with PFEA. Finally, cells were resuspended in fixation solution (PFEA, 1% formaldehyde) and kept at 4° C. before analysis using an iQue Screener (Intellicyt). T2 cells were gated based on the FSC-A/SSC-A signal and doublets removed using an FSC-H/FSC-A doublet exclusion. The PE channel positive gate coordinates were based on an unstained control. Data was plotted using GraphPad Prism v7.

### 15. Sequence Alignment

Multiple sequence alignments were performed by using Clustal Omega Multiple Sequence Alignment (www.ebi.ac.uk/Tools/msa/clustalo/) (Madeira et al. “The EMBL-EBI search and sequence analysis tools APIs in 2019”, Nucleic Acids Research, 47:W636-W641, 2019, doi: 10.1093/nar/gkz268).

### 16. Statistical Analysis

All data were plotted using the GraphPad Prism software version 7. Correlation between x and y datasets were calculated by computing the Pearson correlation coefficient and were reported as R² using the GraphPad Prism software version 7. R² and X² values for curve fittings of biolayer interferometry binding kinetics measurements were calculated using the Octet RED384 system software DataAnalysis HT version 10.0.3.7.

### 17. Design and Production of Disulfide-Stabilized Empty HLA-A*02:01 Molecules

Molecular dynamics simulations of empty and peptide loaded MHC class I molecules have indicated that the former has an increased mobility in the F-pocket that accommodates the C-terminus of the peptide ligand (16). In previous studies with the murine MHC class I molecule H-2K^{b} introduction of a disulfide bond between opposing residues in the F-pocket by mutating a tyrosine at position 84 and an alanine at position 139 to cysteines was able to stabilize the complex. The mutant could be refolded without full length peptide and was capable of retroactive peptide binding (17, 18).

The inventors hypothesized that the same concept could be applied to the human MHC class I molecule HLA-A*02:01. Modifications resulting in mutations of the tyrosine at position 84 and alanine at position 139 into cysteines were introduced into an HLA-A*02:01 heavy chain expression plasmid. After production as inclusion bodies in E. coli, the heavy chain was incubated with similarly produced β₂m but without peptide in refolding buffer. After size exclusion chromatography (SEC), no HLA-A*02:01 associated monomer fraction could be observed compared to a wild type control refolded with a 9mer peptide.

In a second approach, the dipeptide GM was added to the refolding: This dipeptide has a very low affinity for the MHC class I complex and assists the refolding (19). During SEC it dissociates quickly from the binding pocket by buffer exchange against the running buffer, yielding purified empty disulfide-stabilized Y84C/A139C HLA-A*02:01. Empty wild type A*02:01 complexes (WT-A*02:01) could not be produced in the same fashion. WT-A*02:01 complexes can be produced with the dipeptide but denature when attempting to remove the dipeptide by buffer exchange.

The inventors also introduced modifications resulting in mutations of phenylalanine at position 22 and serine at position 71 into cysteines into an HLA-A*02:01 heavy chain expression plasmid. After production as inclusion bodies in E. coli, the heavy chain was incubated with similarly produced β₂m but without peptide in refolding buffer. SEC yielded purified empty disulfide-stabilized F22C/S71C HLA-A*02:01 complexes. The inventors also introduced modifications resulting in mutations of phenylalanine at position 22 and serine at position 71 as well as tryptophan at position 51 and glycine at position 175 into cysteines into an HLA-A*02:01 heavy chain expression plasmid. After production as inclusion bodies in E. coli, the heavy chain was incubated with similarly produced β₂m but without peptide in refolding buffer. SEC yielded purified empty disulfide-stabilized F22C/S71C W51C/G175C HLA-A*02:01 complexes.

The absence of the dipeptide GM in the purified monomer could be shown by thermal stability analysis through buffer exchange: the empty Y84C/A139C HLA-A*02:01 molecule was less temperature stable (i.e., had a lower melting temperature) than the same molecule still complexed with dipeptide GM (41).

The resulting molecules were either biotinylated at 4° C. overnight and separated from excess biotin by a second SEC run or stored directly at −80° C. prior to use.

### 18. Peptide Loading and Affinity Measurements Using Soluble TCRs and Wild Type or Disulfide-Modified MHCs

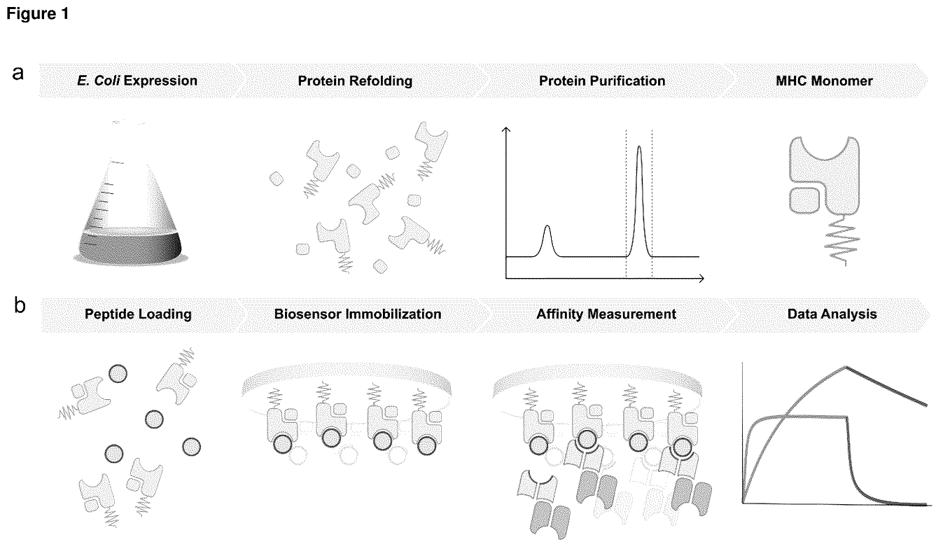

Next, the inventors determined whether the disulfide-modified HLA-A*02:01 molecules were capable of peptide-MHC complex formation and TCR ligand binding. Affinity measurements were performed by bio-layer interferometry (BLI) on an OctetRED 384 using the refolded TCR 1G4 as soluble analyte. This TCR recognizes the HLA-A*02:01 specific peptide SLLMWITQC (ESO 9C, SEQ ID NO: 3) derived from the cancer testis antigen NY-ESO-1 or its synthetic variant SLLMWITQV (ESO 9V, SEQ ID NO: 4) (20,21). Biotinylated Y84C/A139C HLA-A*02:01 was either immobilized directly in its empty state or after a short incubation with the peptide ESO 9V on streptavidin-coated biosensors (FIG. 1b). No differences could be detected between peptide incubations of 5 minutes, the minimal time needed to initiate the affinity measurements after assembly, or longer. Further analysis indicated that full exchange was indeed reached within one to two minutes when high peptide concentrations were used. Kinetics were measured across multiple 1G4 concentrations and wild type HLA-A*02:01 directly refolded with ESO 9V served as control.

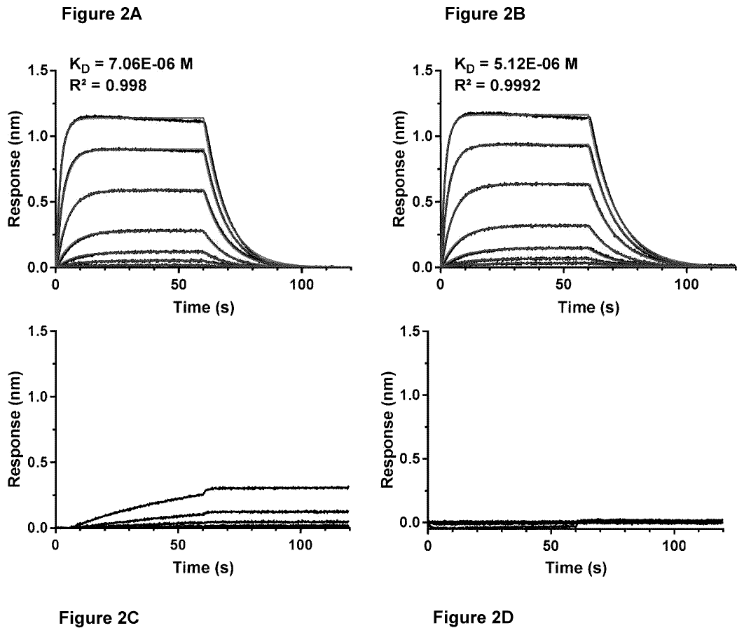

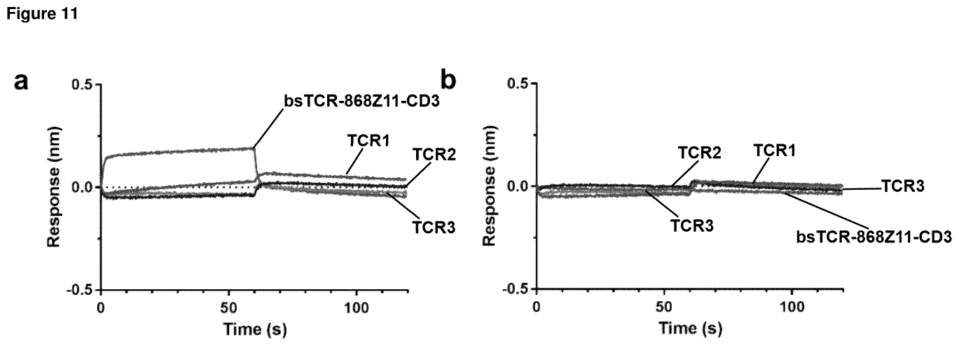

1G4 TCR binding to either Y84C/A139C HLA-A*02:01 9V or WT-A*02:01 ESO 9V was very similar with respect to sensorgrams and K_{d}s resulting from curve fittings (FIG. 2A and 2B). A weak binding signal (but no dissociation) could be detected for the empty immobilized monomer at high concentrations of 1G4 (FIG. 2C). This binding could be prevented by subsequently adding a peptide that is not recognized by 1G4 like SLYNTVATL (FIG. 2D, SEQ ID NO: 5). The weak signal obtained with empty Y84C/A139C HLA-A*02:01 might be explained by unspecific interactions of the TCR with the empty binding pocket, a state that is typically not encountered by TCRs in vivo. Other A*02:01-restricted soluble TCRs with varying specificities behaved similarly, showing no binding to irrelevantly loaded Y84C/A139C HLA-A*02:01 pMHCs but association to functionally empty molecules, albeit but with a relatively lower response (FIG. 11).

### 19. Correlation Between Disulfide-Modified HLA-A*02:01 and WT-A*02:01 Affinity Measurements for an Affinity Maturated TCR

Having established the usability of the Y84C/A139C HLA-A*02:01 molecule as ligand equivalent to WT-A*02:01 for unmodified TCRs the inventors wanted to expand this analysis towards mutated high affinity TCRs and a larger number of peptide ligands. The inventors employed the maturated single chain TCR (scTv) 868Z11, an affinity maturated variant of a TCR that recognizes the HIV p17 Gag-derived HLA-A*02:01 restricted peptide SLYNTVATL (SL9, SEQ ID NO: 5) (8, 22).

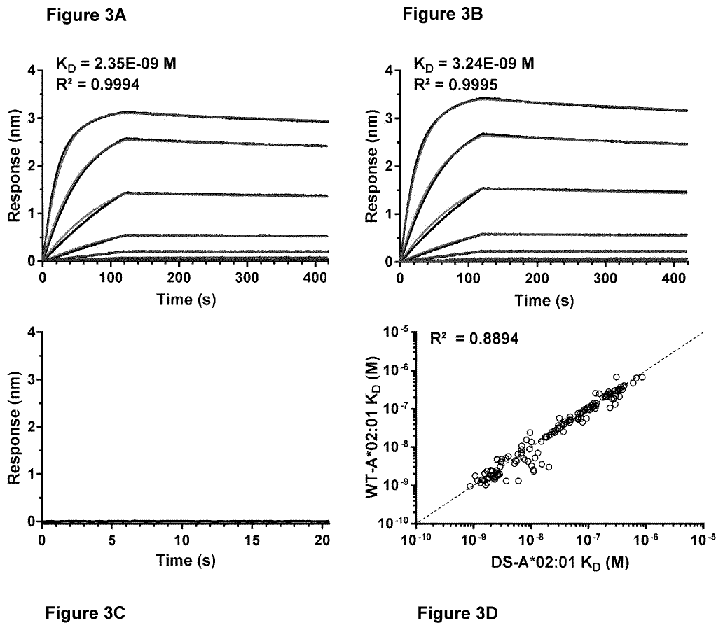

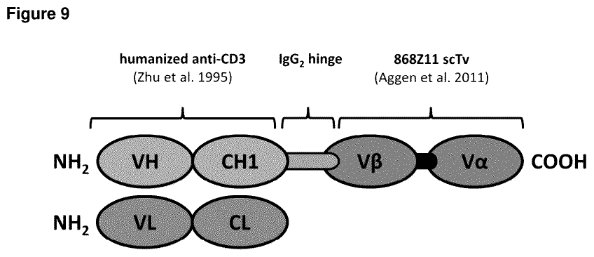

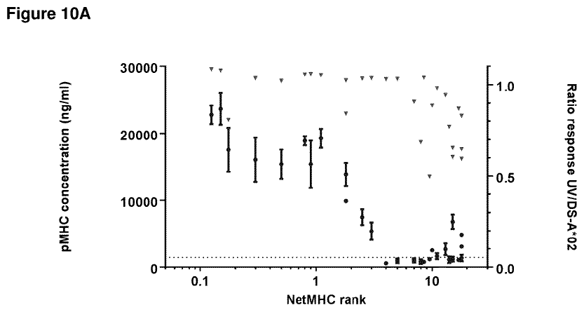

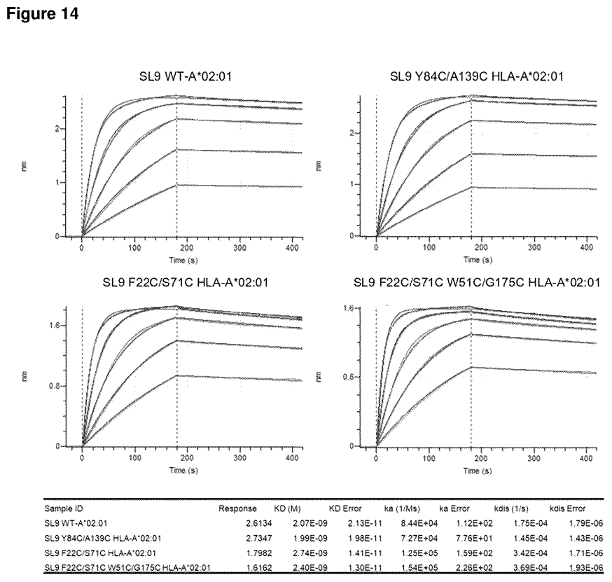

The inventors performed affinity measurements by immobilization of empty or SL9 peptide loaded disulfide-modified HLA-A*02:01 molecules on streptavidin biosensor and measurements against soluble bs-868Z11-CD3, a bsTCR variant of the 868Z11 scTv expressed in fusion with a humanised anti-CD3 antibody (FIG. 9) (23). Binding affinity for SL9 disulfide-modified HLA-A*02:01 pMHC complexes using either Y84C/A139C HLA-A*02:01, F22C/S71C HLA-A*02:01 or F22C/S71C W51C/G175C HLA-A*02:01, was similar to the SL9 WT-A*02:01 pMHC produced by performing an UV-light mediated peptide ligand exchange (25) with 2.35 nM and 3.24 nM, respectively (FIG. 3a and, also FIG. 14). No binding was measurable with empty MHC molecules for this bsTCR (FIG. 3c) and with irrelevantly loaded Y84C/A139C HLA-A*02:01 complexes at a high molar concentrations of 13.3 μM.

Next, the inventors analysed bs-868Z11-CD3 binding affinities towards a positional scanning library based on the SL9 peptide sequence. This library was created by exchanging an amino acid at one position of the wild type SL9 peptide against the 18 remaining proteinogenic amino acids while maintaining all other positions, resulting in 162 distinct peptides when performed at all positions of the nonamer (cysteine was excluded because of its propensity to dimerize) (24). pMHC complexes were generated by the inventors either by addition to Y84C/A139C HLA-A*02:01 molecules as before or by performing UV-light mediated peptide ligand exchange, a technology used for pMHC complex generation (25). Respective pMHC complexes were immobilized on streptavidin and kinetics measured at two different bs-868Z11-CD3 concentrations. As expected, using alternated peptide ligands resulted in a wide range of different K_{d}s, ranging from undetectable within the sensitivity limits of the chosen setup to comparable or even stronger than the interaction with the unmodified SL9 peptide.

For direct comparison, all measured pMHC complexes were selected that had evaluable signals at both analyte concentrations and curve fittings with R² values of at least 0.9, representative of signals within the selected K_{d} sensitivity range. K_{d} values for the resulting 140 peptide ligands were very similar across the whole affinity range when plotted against each other, a finding supported by the high correlation coefficient value (FIG. 3d). Discrepancies were within 2-fold range for over 90% of the pMHC pairs and 6.82-fold differences at most. Within the group with higher than 2-fold changes a trend towards a larger dissociation constant for measurements with the Y84C/A139C HLA-A*02:01 molecule was observed.

The amount of functional pMHC immobilized on each biosensor expressed by the reported Rₘₐₓ value for 140 different peptide ligands from the positional scanning library was comparable for both wild-type and disulfide-stabilized pMHCs (correlation coefficient R²=0.9459).

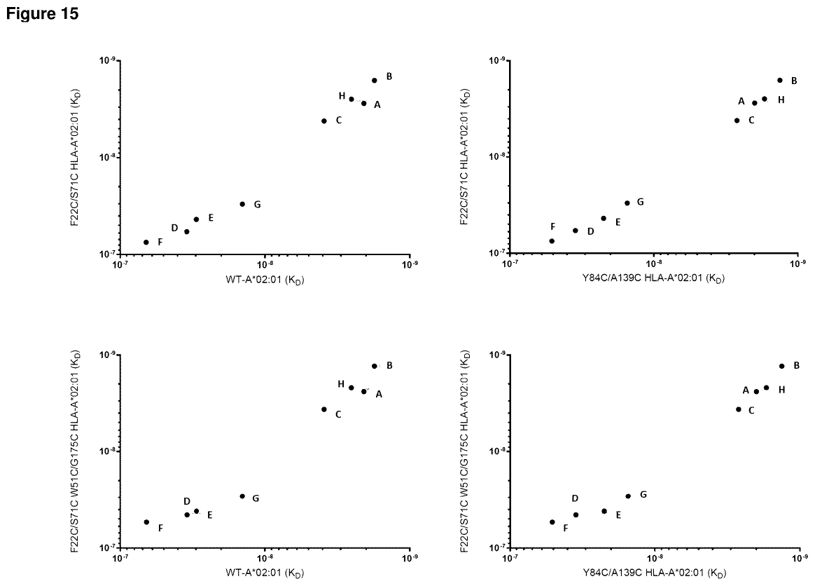

FIG. 15 shows K_{d} values of a high affinity TCR to different pMHC complexes. In each case the K_{d} of the WT-A*02:01 molecules or the Y84C/A139C HLA-A*02:01 molecule is shown on the X-axis and the K_{d} of the two different disulfide-modified HLA-A*02:01 MHC molecules is shown on the y-axis and each dot represents one of different peptides loaded in the MHC molecule. In each square in FIG. 15 the following peptides are represented:

A: HIV-005 WT
(SLYNTVATL, SEQ ID NO: 5)
B: HIV-005 6I
(SLYNTIATL, SEQ ID NO: 110)
C: HIV-005 8V
(SLYNTVAVL, SEQ ID NO: 145)
D: HIV-005 3F
(SLFNTVATL, SEQ ID NO: 59)
E: HIV-005 3F6I8V
(SLFNTIAVL), SEQ ID NO: 318)
F: HIV-005 3F8V
(SLFNTVAVL, SEQ ID NO: 319)
G: HIV-005 3F6I
(SLFNTIATL, SEQ ID NO: 320)
H: HIV-005 6I8V
(SLYNTIAVL, SEQ ID NO: 321)

In the upper left panel the K_{d} for each above-listed peptide for the WT-A*02:01 pMHC complex is plotted against the K_{d} of the disulfide-modified F22C/S71C HLA-A*02:01 pMHC complex. The disulfide-modified F22C/S71C HLA-A*02:01 pMHC complex shows almost identical K_{D} values to the WT-A*02:01 pMHC complex for each of the investigated peptides. In the lower left panel the K_{d} for each above-listed peptide for the WT-A*02:01 pMHC complex is plotted against the K_{D} of the disulfide-modified F22C/S71C W51C/G175C HLA-A*02:01 pMHC complex and shows also almost identical K_{d} values to the WT-A*02:01 pMHC complex for each of the investigated peptides.

In the upper right panel the K_{d} for each above-listed peptide for the Y84C/A139C HLA-A*02:01 pMHC complex is plotted against the K_{d} of the disulfide-modified F22C/S71C HLA-A*02:01 pMHC complex. In the lower right panel the K_{d} for each above-listed peptide for the Y84C/A139C HLA-A*02:01 pMHC complex is plotted against the K_{d} of the disulfide-modified F22C/S71C W51C/G175C HLA-A*02:01 pMHC complex. The disulfide-modified pMHC complexes of the F22C/S71C and the F22C/S71C W51C/G175C mutant have almost identical K_{d} values compared to the Y84C/A139C HLA-A*02:01 pMHC complex for each of the investigated peptides. It can thus, be concluded that disulfide-modified HLA-A*02:01 molecules loaded with different peptides and forming pMHC complexes are comparably recognized by a respective affinity-maturated TCR to the WT HLA-A*02:01 pMHC complex. Therefore, the function of the disulfide-modified HLA-A*02:01 molecules loaded with peptides (pMHC complexes) is unaffected by the introduction of stabilizing amino acid mutations into the HLA-A*02:01 molecule.

The results shown in FIG. 15 make it credible for the skilled person that the disulfide-modified HLA-A*02:01 molecules according to the present invention loaded with peptide ligands and forming disulfide-modified pMHC complexes elicit a T-cell response upon binding to their respective TCR.

### 20. High-Throughput Kinetic Screenings for Binding Motif Generation

Quick and flexible generation of pMHCs facilitates the collection of large binding affinity datasets against many different pMHCs. One example of such a dataset is screening of a positional scanning library to generate a pMHC-bsTCR binding motif, which can serve as one component in a bsTCR safety screening approach. To perform such measurements, the pMHC should ideally be used as a soluble analyte because this offers multiple advantages. First, immobilizing the same ligand with known activity repeatedly, for example, a bsTCR, allows better interpretation of the fitting results, especially the reported Rₘₐₓ value. Second, using pMHC complexes as soluble analytes instead of immobilizing is preferable for quick and cost effective high throughput screenings, since a broad variety of regeneratable biosensors capable of reversibly immobilizing bispecific TCR constructs exists. These biosensors are typically coated with antibodies and can be used at least 20 times for kinetic measurements without loss of readout quality. Third, immobilizing the bsTCR is the only orientation available for measuring monovalent affinity when a bsTCR or antibody has multiple pMHC binding moieties, because, with immobilized pMHCs, only avidity can be measured.

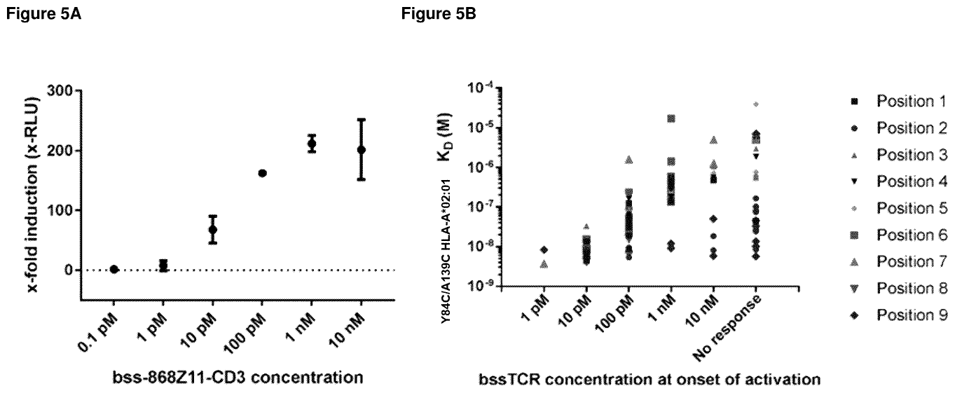

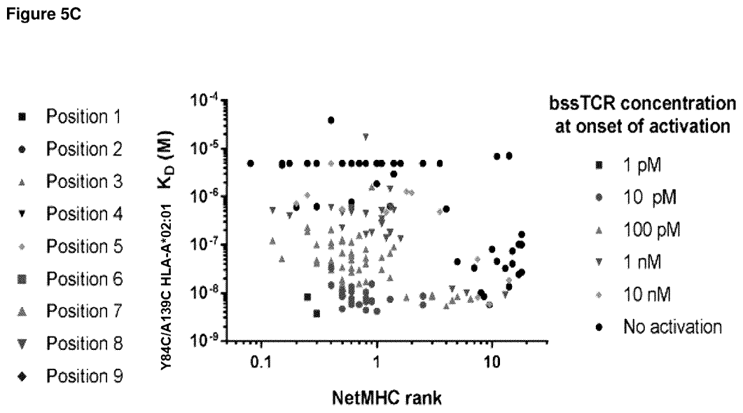

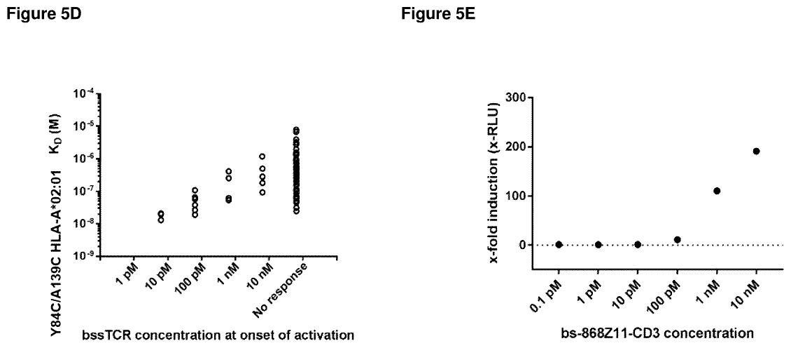

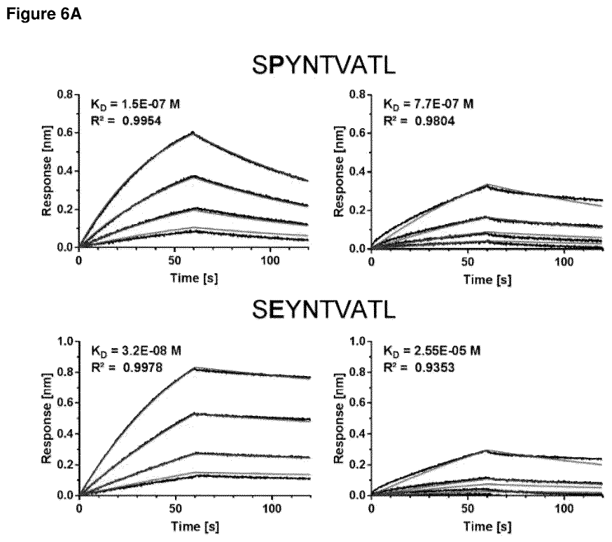

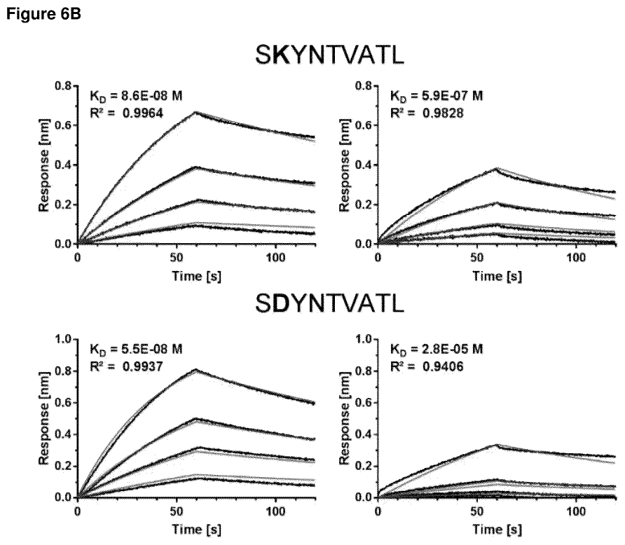

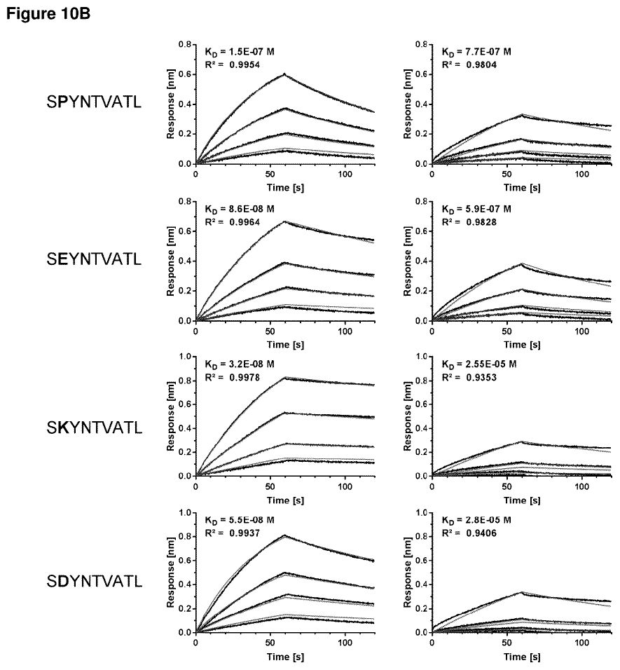

While the UV mediated peptide ligand exchange can generate a high number of different pMHC complexes, the exchange efficiency varies depending on the peptide and its affinity for binding to the respective MHC class I allele, resulting in different pMHC concentrations in the samples (FIG. 10). This uncertainty is a problem for affinity measurements with pMHCs used as soluble analytes, as precise knowledge of the concentration is desired to determine accurate affinities. Since the disulfide-stabilized Y84C/A139C HLA-A*02:01 mutant is stable without any peptide, this restriction does not apply. If the peptides are added at a concentration high enough to saturate the empty MHC complexes, the effective concentration of pMHC is known, significantly increasing the accuracy of the measurements and avoiding false negatives. Examples for this behavior could be detected in the positional scanning library, resulting in bad fitting data and miscalculation of the affinity when UV exchange preparations were used compared to Y84C/A139C HLA-A*02:01 peptide loadings (FIGS. 5, 6, 10) (26). Accurately measuring bsTCR affinities for such peptides can be important in the context of binding motif generations, because these substitutions may result in relevant MHC binders when combined with substitutions at other positions. Tolerance of the amino acids by the bsTCR should thus, be reflected correctly in a comprehensive binding motif.

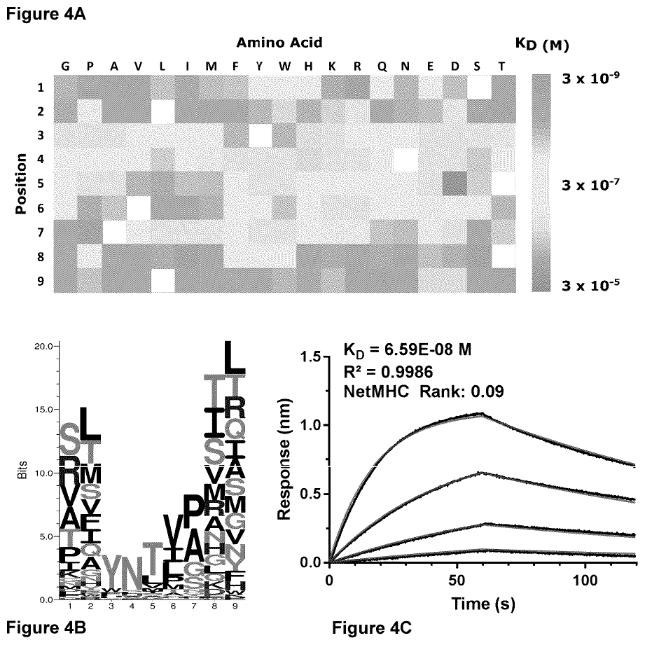

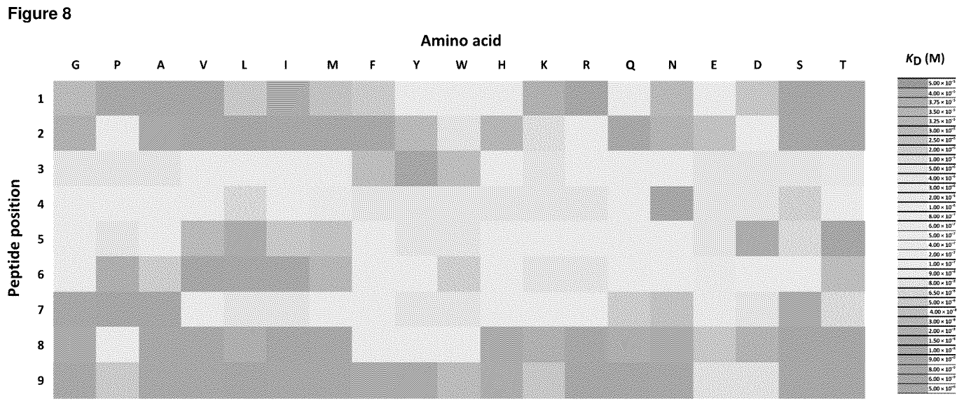

By immobilizing the bs-868Z11-CD3 bsTCR the inventors were able to analyze the positional scanning library at four different soluble pMHC concentrations for each peptide ligand, ranging from 500 to 15.8 nM, within 4 hours of unattended measurement time at a 20-fold reduced price tag. All curves reaching at least a signal level of 0.05 nm were included in the fittings, resulting in a comprehensive TCR binding motif (FIGS. 4a, 8, Table 3).

TABLE 3
bs-868Z11-CD3 binding affinity for SV9 peptide SLYNTVATL (SEQ ID NO: 5)
and peptides from positional scanning library (SEQ ID NOS: 16-177). Table
includes K_{D}, kₒₙ and k_{off} values determined by curve fittings following a 1:1
Langmuir binding model using the Fortébio Data Analysis HT 10.0.3.7 software.
Respective errors are reported as well as accuracy of the fit according to the
model. Peptides reported as ″No fit″ had no evaluable curves reaching at least
a peak signal of 0.05 nm at any concentration.

| | | | kₒₙ | kₒₙ | k_{off} | k_{off} | | |
|---|---|---|---|---|---|---|---|---|
| Peptide | KD (M) | KD Error | (M⁻¹s⁻¹) | Error | (s⁻¹) | Error | Full X² | Full R² |
| SLYNTVATL | 3.81E−09 | 1.49E−10 | 1.03E+05 | 1.45E+02 | 3.91E−04 | 1.53E−05 | 0.262 | 0.9993 |
| GLYNTVATL | 3.05E−08 | 3.55E−10 | 1.04E+05 | 3.42E+02 | 3.19E−03 | 3.56E−05 | 1.0915 | 0.9966 |
| PLYNTVATL | 8.54E−09 | 3.46E−10 | 9.65E+04 | 3.03E+02 | 8.24E−04 | 3.33E−05 | 1.2363 | 0.9969 |
| ALYNTVATL | 5.82E−09 | 3.18E−10 | 1.04E+05 | 3.14E+02 | 6.04E−04 | 3.29E−05 | 1.2791 | 0.9969 |
| VLYNTVATL | 5.74E−09 | 2.24E−10 | 1.05E+05 | 2.27E+02 | 6.04E−04 | 2.35E−05 | 0.6719 | 0.9984 |
| LLYNTVATL | 4.99E−08 | 2.99E−10 | 1.04E+05 | 2.67E+02 | 5.17E−03 | 2.80E−05 | 0.5623 | 0.9981 |
| ILYNTVATL | 1.35E−08 | 2.35E−10 | 1.06E+05 | 2.40E+02 | 1.43E−03 | 2.47E−05 | 0.6748 | 0.9982 |
| MLYNTVATL | 4.19E−08 | 2.95E−10 | 1.09E+05 | 2.93E+02 | 4.56E−03 | 2.96E−05 | 0.6922 | 0.9978 |
| FLYNTVATL | 5.22E−08 | 3.07E−10 | 1.15E+05 | 3.20E+02 | 6.02E−03 | 3.13E−05 | 0.6452 | 0.9976 |
| YLYNTVATL | 1.24E−07 | 5.65E−10 | 1.15E+05 | 4.01E+02 | 1.43E−02 | 4.21E−05 | 0.4931 | 0.9972 |
| WLYNTVATL | 4.62E−07 | 4.57E−09 | 1.66E+05 | 1.48E+03 | 7.66E−02 | 3.27E−04 | 0.1216 | 0.9955 |
| HLYNTVATL | 3.43E−07 | 2.50E−09 | 1.34E+05 | 8.87E+02 | 4.60E−02 | 1.43E−04 | 0.1908 | 0.9961 |
| KLYNTVATL | 1.91E−08 | 2.14E−10 | 9.03E+04 | 1.67E+02 | 1.73E−03 | 1.91E−05 | 0.3239 | 0.999 |
| RLYNTVATL | 4.42E−09 | 5.15E−10 | 8.86E+04 | 3.93E+02 | 3.92E−04 | 4.56E−05 | 2.0699 | 0.9944 |
| QLYNTVATL | 1.38E−07 | 5.55E−10 | 9.85E+04 | 3.05E+02 | 1.36E−02 | 3.50E−05 | 0.3534 | 0.9981 |
| NLYNTVATL | 3.13E−08 | 3.42E−10 | 9.84E+04 | 2.98E+02 | 3.08E−03 | 3.23E−05 | 0.9466 | 0.9973 |
| ELYNTVATL | 4.85E−07 | 4.84E−09 | 9.29E+04 | 8.66E+02 | 4.50E−02 | 1.63E−04 | 0.4525 | 0.9948 |
| DLYNTVATL | 4.49E−08 | 3.46E−10 | 9.55E+04 | 2.77E+02 | 4.28E−03 | 3.06E−05 | 0.7816 | 0.9977 |
| TLYNTVATL | 6.94E−09 | 2.07E−10 | 1.02E+05 | 1.98E+02 | 7.07E−04 | 2.10E−05 | 0.544 | 0.9988 |
| SGYNTVATL | 1.86E−08 | 4.56E−10 | 8.40E+04 | 3.18E+02 | 1.56E−03 | 3.79E−05 | 1.3876 | 0.9964 |
| SPYNTVATL | 1.65E−07 | 2.27E−09 | 6.80E+04 | 7.29E+02 | 1.12E−02 | 9.66E−05 | 2.2025 | 0.9852 |
| SAYNTVATL | 1.00E−08 | 1.28E−10 | 1.02E+05 | 1.23E+02 | 1.02E−03 | 1.30E−05 | 0.2052 | 0.9995 |
| SVYNTVATL | 8.47E−09 | 1.64E−10 | 1.01E+05 | 1.55E+02 | 8.57E−04 | 1.65E−05 | 0.3327 | 0.9992 |
| SIYNTVATL | 8.68E−09 | 9.77E−11 | 1.02E+05 | 9.42E+01 | 8.89E−04 | 9.97E−06 | 0.1192 | 0.9997 |
| SMYNTVATL | 6.55E−09 | 2.07E−10 | 1.01E+05 | 1.95E+02 | 6.61E−04 | 2.08E−05 | 0.4808 | 0.9987 |
| SFYNTVATL | 8.52E−09 | 3.97E−10 | 9.54E+04 | 3.41E+02 | 8.13E−04 | 3.77E−05 | 1.5251 | 0.996 |
| SYYNTVATL | 3.26E−08 | 3.90E−10 | 5.83E+04 | 1.62E+02 | 1.90E−03 | 2.21E−05 | 0.23 | 0.9989 |
| SWYNTVATL | 8.16E−08 | 1.74E−09 | 4.46E+04 | 4.66E+02 | 3.64E−03 | 6.77E−05 | 0.9827 | 0.991 |
| SHYNTVATL | 2.73E−08 | 8.86E−10 | 6.92E+04 | 4.66E+02 | 1.89E−03 | 5.99E−05 | 2.1947 | 0.9915 |
| SKYNTVATL | 7.43E−08 | 1.57E−09 | 5.08E+04 | 5.00E+02 | 3.77E−03 | 7.06E−05 | 2.0162 | 0.9899 |
| SRYNTVATL | 1.02E−07 | 2.33E−09 | 4.84E+04 | 6.42E+02 | 4.95E−03 | 9.17E−05 | 0.6946 | 0.9837 |
| SQYNTVATL | 9.41E−09 | 2.19E−10 | 1.09E+05 | 2.35E+02 | 1.03E−03 | 2.37E−05 | 0.6976 | 0.9984 |
| SNYNTVATL | 2.45E−08 | 6.68E−10 | 6.85E+04 | 3.45E+02 | 1.68E−03 | 4.50E−05 | 1.7367 | 0.9953 |
| SEYNTVATL | 4.09E−08 | 1.77E−09 | 5.16E+04 | 6.23E+02 | 2.11E−03 | 8.78E−05 | 4.5691 | 0.9843 |
| SDYNTVATL | 1.01E−07 | 1.68E−09 | 6.51E+04 | 6.61E+02 | 6.56E−03 | 8.69E−05 | 3.2507 | 0.9854 |
| SSYNTVATL | 8.17E−09 | 1.97E−10 | 9.64E+04 | 1.72E+02 | 7.88E−04 | 1.89E−05 | 0.4063 | 0.999 |
| STYNTVATL | 5.41E−09 | 1.49E−10 | 9.87E+04 | 1.35E+02 | 5.34E−04 | 1.47E−05 | 0.2427 | 0.9994 |
| SLGNTVATL | No fit | | | | | | | |
| SLPNTVATL | No fit | | | | | | | |
| SLANTVATL | No fit | | | | | | | |
| SLVNTVATL | 5.11E−07 | 5.80E−09 | 1.95E+05 | 2.01E+03 | 9.96E−02 | 4.80E−04 | 0.0769 | 0.9966 |
| SLLNTVATL | 1.32E−07 | 8.45E−10 | 1.21E+05 | 6.09E+02 | 1.60E−02 | 6.33E−05 | 1.038 | 0.9944 |
| SLINTVATL | 4.77E−07 | 5.50E−09 | 1.40E+05 | 1.48E+03 | 6.69E−02 | 3.15E−04 | 0.325 | 0.9939 |
| SLMNTVATL | 1.07E−06 | 5.52E−08 | 2.35E+05 | 1.12E+04 | 2.50E−01 | 5.13E−03 | 0.1244 | 0.979 |
| SLFNTVATL | 3.47E−08 | 1.92E−10 | 9.54E+04 | 1.59E+02 | 3.31E−03 | 1.75E−05 | 0.2445 | 0.9992 |
| SLWNTVATL | 3.36E−08 | 1.91E−10 | 9.34E+04 | 1.53E+02 | 3.14E−03 | 1.71E−05 | 0.2479 | 0.9992 |
| SLHNTVATL | 9.09E−08 | 3.31E−10 | 1.16E+05 | 2.84E+02 | 1.06E−02 | 2.85E−05 | 0.3676 | 0.9984 |
| SLKNTVATL | No fit | | | | | | | |
| SLRNTVATL | 5.55E−07 | 5.54E−09 | 9.64E+04 | 9.00E+02 | 5.35E−02 | 1.88E−04 | 0.1675 | 0.9957 |
| SLQNTVATL | 6.29E−07 | 9.45E−09 | 2.62E+05 | 3.51E+03 | 1.65E−01 | 1.11E−03 | 0.0384 | 0.9961 |
| SLNNTVATL | 4.74E−07 | 5.90E−09 | 1.81E+05 | 2.05E+03 | 8.59E−02 | 4.48E−04 | 0.1049 | 0.9953 |
| SLENTVATL | No fit | | | | | | | |
| SLDNTVATL | No fit | | | | | | | |
| SLSNTVATL | No fit | | | | | | | |
| SLTNTVATL | 3.01E−06 | 3.45E−06 | 1.88E+05 | 2.15E+05 | 5.66E−01 | 6.50E−02 | 0.1123 | 0.9199 |
| SLYGTVATL | 5.33E−07 | 1.20E−08 | 2.07E+05 | 4.15E+03 | 1.11E−01 | 1.12E−03 | 0.5678 | 0.9842 |
| SLYPTVATL | 5.54E−07 | 1.44E−08 | 3.62E+05 | 8.18E+03 | 2.00E−01 | 2.58E−03 | 0.0884 | 0.9892 |
| SLYATVATL | 1.15E−07 | 6.46E−10 | 1.40E+05 | 6.09E+02 | 1.60E−02 | 5.72E−05 | 0.9354 | 0.9952 |
| SLYVTVATL | 1.80E−07 | 9.65E−10 | 1.31E+05 | 5.98E+02 | 2.35E−02 | 6.64E−05 | 0.4669 | 0.9962 |
| SLYLTVATL | 6.70E−08 | 3.12E−10 | 1.11E+05 | 2.85E+02 | 7.44E−03 | 2.89E−05 | 0.5152 | 0.9981 |
| SLYITVATL | 5.25E−07 | 5.68E−09 | 1.18E+05 | 1.18E+03 | 6.19E−02 | 2.55E−04 | 0.2208 | 0.9949 |
| SLYMTVATL | 1.88E−06 | 2.10E−06 | 3.58E+05 | 3.96E+05 | 6.72E−01 | 1.11E−01 | 0.09 | 0.876 |
| SLYFTVATL | No fit | | | | | | | |
| SLYYTVATL | No fit | | | | | | | |
| SLYWTVATL | No fit | | | | | | | |
| SLYHTVATL | 8.11E−08 | 6.16E−10 | 1.38E+05 | 7.03E+02 | 1.12E−02 | 6.26E−05 | 1.8422 | 0.9923 |
| SLYKTVATL | No fit | | | | | | | |
| SLYRTVATL | No fit | | | | | | | |
| SLYQTVATL | 2.84E−07 | 3.23E−09 | 1.71E+05 | 1.73E+03 | 4.86E−02 | 2.53E−04 | 0.6721 | 0.9898 |
| SLYETVATL | No fit | | | | | | | |
| SLYDTVATL | No fit | | | | | | | |
| SLYSTVATL | 5.95E−08 | 2.69E−10 | 1.27E+05 | 3.13E+02 | 7.57E−03 | 2.87E−05 | 0.5404 | 0.9981 |
| SLYTTVATL | 1.61E−07 | 7.59E−10 | 1.35E+05 | 5.34E+02 | 2.18E−02 | 5.63E−05 | 0.3965 | 0.9968 |
| SLYNGVATL | 6.03E−07 | 1.50E−07 | 5.42E+05 | 1.29E+05 | 3.27E−01 | 2.34E−02 | 0.0452 | 0.9399 |
| SLYNPVATL | No fit | | | | | | | |
| SLYNAVATL | 9.66E−08 | 5.82E−10 | 1.27E+05 | 5.41E+02 | 1.22E−02 | 5.20E−05 | 0.9944 | 0.995 |
| SLYNVVATL | 3.07E−08 | 4.63E−10 | 9.09E+04 | 3.58E+02 | 2.79E−03 | 4.06E−05 | 1.3333 | 0.9958 |
| SLYNLVATL | 1.46E−08 | 3.05E−10 | 9.80E+04 | 2.73E+02 | 1.43E−03 | 2.97E−05 | 0.917 | 0.9976 |
| SLYNIVATL | 4.85E−08 | 3.37E−10 | 8.41E+04 | 2.21E+02 | 4.08E−03 | 2.62E−05 | 0.4314 | 0.9984 |
| SLYNMVATL | 4.26E−08 | 3.52E−10 | 1.20E+05 | 4.09E+02 | 5.12E−03 | 3.86E−05 | 1.2415 | 0.9962 |
| SLYNFVATL | 7.26E−07 | 3.75E−08 | 2.97E+05 | 1.36E+04 | 2.16E−01 | 5.09E−03 | 0.3346 | 0.9697 |
| SLYNYVATL | 4.60E−06 | 9.88E−06 | 1.37E+05 | 2.94E+05 | 6.31E−01 | 9.34E−02 | 0.1113 | 0.8904 |
| SLYNWVATL | No fit | | | | | | | |
| SLYNHVATL | 6.23E−07 | 4.07E−08 | 4.00E+05 | 2.26E+04 | 2.49E−01 | 8.17E−03 | 0.2106 | 0.9526 |
| SLYNKVATL | 2.24E−07 | 1.05E−09 | 1.35E+05 | 5.55E+02 | 3.02E−02 | 6.82E−05 | 0.2572 | 0.9973 |
| SLYNRVATL | 7.78E−07 | 7.24E−08 | 3.54E+05 | 3.19E+04 | 2.76E−01 | 6.54E−03 | 0.02 | 0.9899 |
| SLYNQVATL | 4.72E−07 | 7.25E−09 | 2.10E+05 | 2.90E+03 | 9.91E−02 | 6.64E−04 | 0.131 | 0.9936 |
| SLYNNVATL | 1.19E−07 | 5.68E−10 | 1.32E+05 | 4.88E+02 | 1.58E−02 | 4.76E−05 | 0.5956 | 0.9966 |
| SLYNEVATL | No fit | | | | | | | |
| SLYNDVATL | 3.91E−05 | 5.33E−04 | 1.23E+04 | 1.67E+05 | 4.79E−01 | 5.67E−02 | 0.1685 | 0.904 |
| SLYNSVATL | 6.91E−08 | 3.75E−10 | 1.21E+05 | 3.86E+02 | 8.39E−03 | 3.68E−05 | 0.7181 | 0.997 |
| SLYNTGATL | 1.34E−07 | 6.48E−10 | 1.37E+05 | 5.33E+02 | 1.84E−02 | 5.26E−05 | 0.5267 | 0.9965 |
| SLYNTPATL | 1.54E−08 | 1.56E−10 | 1.19E+05 | 1.93E+02 | 1.83E−03 | 1.82E−05 | 0.3561 | 0.999 |
| SLYNTAATL | 5.48E−08 | 3.74E−10 | 1.10E+05 | 3.59E+02 | 6.05E−03 | 3.62E−05 | 0.8843 | 0.9967 |
| SLYNTLATL | 9.08E−09 | 1.01E−10 | 1.12E+05 | 1.15E+02 | 1.02E−03 | 1.13E−05 | 0.15 | 0.9996 |
| SLYNTIATL | 8.74E−09 | 1.86E−10 | 9.97E+04 | 1.72E+02 | 8.71E−04 | 1.85E−05 | 0.3788 | 0.999 |
| SLYNTMATL | 2.72E−08 | 3.66E−10 | 9.75E+04 | 3.17E+02 | 2.65E−03 | 3.46E−05 | 0.9551 | 0.9966 |
| SLYNTFATL | 5.79E−07 | 6.47E−09 | 7.96E+04 | 8.39E+02 | 4.61E−02 | 1.69E−04 | 0.1416 | 0.9946 |
| SLYNTYATL | 4.43E−07 | 8.76E−09 | 4.61E+04 | 8.61E+02 | 2.04E−02 | 1.33E−04 | 0.1286 | 0.9831 |
| SLYNTWATL | 1.74E−05 | 1.28E−05 | 1.91E+03 | 1.41E+03 | 3.33E−02 | 2.40E−04 | 0.0063 | 0.9878 |
| SLYNTHATL | 1.75E−07 | 1.46E−09 | 7.39E+04 | 4.94E+02 | 1.30E−02 | 6.42E−05 | 0.3653 | 0.9929 |
| SLYNTKATL | No fit | | | | | | | |
| SLYNTRATL | No fit | | | | | | | |
| SLYNTQATL | 2.71E−07 | 1.50E−09 | 1.19E+05 | 5.86E+02 | 3.22E−02 | 7.94E−05 | 0.1392 | 0.9969 |
| SLYNTNATL | 1.79E−07 | 7.80E−10 | 1.20E+05 | 4.42E+02 | 2.15E−02 | 5.03E−05 | 0.2983 | 0.9974 |
| SLYNTEATL | 1.43E−06 | 5.11E−08 | 5.54E+04 | 1.93E+03 | 7.94E−02 | 5.83E−04 | 0.0772 | 0.99 |
| SLYNTDATL | 6.04E−07 | 7.08E−09 | 1.12E+05 | 1.22E+03 | 6.77E−02 | 2.89E−04 | 0.1139 | 0.995 |
| SLYNTSATL | 1.66E−07 | 6.43E−10 | 1.43E+05 | 4.69E+02 | 2.38E−02 | 4.93E−05 | 0.2673 | 0.9979 |
| SLYNTTATL | 3.37E−08 | 4.58E−10 | 1.07E+05 | 4.51E+02 | 3.59E−03 | 4.64E−05 | 1.7673 | 0.9938 |
| SLYNTVGTL | 8.29E−09 | 4.59E−10 | 8.24E+04 | 3.12E+02 | 6.83E−04 | 3.77E−05 | 1.2283 | 0.996 |
| SLYNTVPTL | 3.71E−09 | 4.42E−10 | 1.22E+05 | 5.76E+02 | 4.51E−04 | 5.36E−05 | 4.052 | 0.9904 |
| SLYNTVVTL | 5.99E−07 | 1.07E−08 | 1.44E+05 | 2.37E+03 | 8.64E−02 | 6.15E−04 | 0.1883 | 0.9892 |
| SLYNTVLTL | No fit | | | | | | | |
| SLYNTVITL | No fit | | | | | | | |
| SLYNTVMTL | 1.02E−07 | 4.28E−10 | 6.90E+04 | 1.80E+02 | 7.04E−03 | 2.31E−05 | 0.1333 | 0.9988 |
| SLYNTVFTL | 5.14E−07 | 1.01E−08 | 1.72E+05 | 3.07E+03 | 8.85E−02 | 7.06E−04 | 0.1274 | 0.9897 |
| SLYNTVYTL | No fit | | | | | | | |
| SLYNTVWTL | No fit | | | | | | | |
| SLYNTVHTL | 1.14E−07 | 2.51E−10 | 8.42E+04 | 1.27E+02 | 9.63E−03 | 1.53E−05 | 0.0763 | 0.9995 |
| SLYNTVKTL | 1.20E−06 | 5.58E−08 | 5.35E+04 | 2.43E+03 | 6.42E−02 | 6.41E−04 | 0.0792 | 0.9775 |
| SLYNTVRTL | 1.28E−06 | 2.41E−08 | 2.49E+04 | 4.61E+02 | 3.20E−02 | 9.10E−05 | 0.0547 | 0.9967 |
| SLYNTVQTL | 5.38E−08 | 7.00E−10 | 6.84E+04 | 3.40E+02 | 3.68E−03 | 4.43E−05 | 0.9296 | 0.9952 |
| SLYNTVNTL | 4.11E−08 | 8.02E−10 | 7.22E+04 | 4.32E+02 | 2.97E−03 | 5.51E−05 | 1.5878 | 0.9921 |
| SLYNTVETL | 1.61E−06 | 2.46E−07 | 5.74E+03 | 8.80E+02 | 9.22E−03 | 1.00E−04 | 0.007 | 0.989 |
| SLYNTVDTL | No fit | | | | | | | |
| SLYNTVSTL | 1.04E−08 | 4.47E−10 | 9.80E+04 | 4.00E+02 | 1.02E−03 | 4.36E−05 | 1.9634 | 0.9944 |
| SLYNTVTTL | 6.90E−08 | 2.99E−10 | 9.23E+04 | 2.09E+02 | 6.37E−03 | 2.36E−05 | 0.2893 | 0.9987 |
| SLYNTVAGL | 1.14E−08 | 1.42E−10 | 1.14E+05 | 1.65E+02 | 1.30E−03 | 1.61E−05 | 0.302 | 0.9992 |
| SLYNTVAPL | 2.34E−07 | 1.20E−09 | 1.35E+05 | 6.11E+02 | 3.16E−02 | 7.71E−05 | 0.3155 | 0.9969 |
| SLYNTVAAL | 8.50E−09 | 1.51E−10 | 1.14E+05 | 1.75E+02 | 9.69E−04 | 1.72E−05 | 0.3504 | 0.9991 |
| SLYNTVAVL | 6.98E−09 | 1.19E−10 | 1.05E+05 | 1.20E+02 | 7.31E−04 | 1.25E−05 | 0.1881 | 0.9995 |
| SLYNTVALL | 1.58E−08 | 1.20E−10 | 9.58E+04 | 1.03E+02 | 1.51E−03 | 1.14E−05 | 0.1259 | 0.9996 |
| SLYNTVAIL | 4.16E−09 | 7.48E−10 | 9.74E+04 | 6.62E+02 | 4.05E−04 | 7.28E−05 | 5.8607 | 0.9834 |
| SLYNTVAML | 7.69E−09 | 5.22E−10 | 9.75E+04 | 4.63E+02 | 7.50E−04 | 5.08E−05 | 2.7181 | 0.9922 |
| SLYNTVAFL | 1.93E−07 | 1.68E−09 | 9.29E+04 | 6.77E+02 | 1.80E−02 | 8.45E−05 | 0.9456 | 0.9906 |
| SLYNTVAYL | 4.00E−07 | 3.75E−09 | 9.61E+04 | 8.32E+02 | 3.85E−02 | 1.39E−04 | 0.2451 | 0.994 |
| SLYNTVAWL | 2.09E−07 | 1.94E−09 | 9.65E+04 | 7.69E+02 | 2.01E−02 | 9.70E−05 | 1.0358 | 0.9893 |
| SLYNTVAHL | 1.09E−08 | 5.55E−10 | 9.19E+04 | 4.47E+02 | 1.00E−03 | 5.07E−05 | 2.6388 | 0.9925 |
| SLYNTVAKL | 1.73E−08 | 2.87E−10 | 1.02E+05 | 2.76E+02 | 1.77E−03 | 2.90E−05 | 0.9054 | 0.9975 |
| SLYNTVARL | 7.93E−09 | 3.98E−10 | 1.06E+05 | 4.06E+02 | 8.37E−04 | 4.19E−05 | 2.1201 | 0.9946 |
| SLYNTVAQL | 1.59E−08 | 6.14E−10 | 1.01E+05 | 5.74E+02 | 1.61E−03 | 6.13E−05 | 4.0059 | 0.9888 |
| SLYNTVANL | 1.08E−08 | 6.43E−10 | 1.01E+05 | 6.03E+02 | 1.09E−03 | 6.46E−05 | 4.5943 | 0.9874 |
| SLYNTVAEL | 4.73E−08 | 2.37E−10 | 9.22E+04 | 1.79E+02 | 4.36E−03 | 2.02E−05 | 0.291 | 0.999 |
| SLYNTVADL | 2.12E−08 | 3.17E−10 | 8.90E+04 | 2.40E+02 | 1.88E−03 | 2.77E−05 | 0.6889 | 0.9979 |
| SLYNTVASL | 4.68E−09 | 2.55E−10 | 1.09E+05 | 2.71E+02 | 5.08E−04 | 2.76E−05 | 0.918 | 0.9977 |
| SLYNTVATG | 7.71E−09 | 4.30E−10 | 1.01E+05 | 4.05E+02 | 7.79E−04 | 4.34E−05 | 2.1199 | 0.9943 |
| SLYNTVATP | 5.03E−08 | 1.57E−09 | 3.41E+04 | 3.34E+02 | 1.72E−03 | 5.08E−05 | 0.6961 | 0.9945 |
| SLYNTVATA | 6.74E−09 | 4.88E−10 | 1.13E+05 | 5.56E+02 | 7.61E−04 | 5.49E−05 | 3.6905 | 0.9904 |
| SLYNTVATV | 8.41E−09 | 6.00E−10 | 1.04E+05 | 5.95E+02 | 8.76E−04 | 6.23E−05 | 4.752 | 0.988 |
| SLYNTVATI | 6.70E−09 | 2.87E−10 | 1.13E+05 | 3.25E+02 | 7.53E−04 | 3.22E−05 | 1.2712 | 0.9968 |
| SLYNTVATM | 7.45E−09 | 2.48E−10 | 9.88E+04 | 2.26E+02 | 7.36E−04 | 2.44E−05 | 0.6922 | 0.9982 |
| SLYNTVATF | 1.19E−08 | 2.46E−10 | 7.18E+04 | 1.37E+02 | 8.51E−04 | 1.76E−05 | 0.2228 | 0.9992 |
| SLYNTVATY | 1.02E−08 | 3.37E−10 | 7.11E+04 | 1.85E+02 | 7.24E−04 | 2.39E−05 | 0.4625 | 0.9985 |
| SLYNTVATW | 3.32E−08 | 5.59E−10 | 3.70E+04 | 1.34E+02 | 1.23E−03 | 2.02E−05 | 0.0824 | 0.9991 |
| SLYNTVATH | 1.37E−08 | 3.64E−10 | 4.75E+04 | 1.19E+02 | 6.51E−04 | 1.72E−05 | 0.089 | 0.9993 |
| SLYNTVATK | 4.57E−08 | 1.20E−09 | 2.70E+04 | 2.00E+02 | 1.23E−03 | 3.11E−05 | 0.0929 | 0.9982 |
| SLYNTVATR | 5.71E−09 | 2.30E−10 | 9.59E+04 | 1.99E+02 | 5.48E−04 | 2.20E−05 | 0.5532 | 0.9986 |
| SLYNTVATQ | 5.88E−09 | 3.12E−10 | 8.96E+04 | 2.41E+02 | 5.27E−04 | 2.79E−05 | 0.7397 | 0.9978 |
| SLYNTVATN | 9.10E−09 | 3.77E−10 | 9.76E+04 | 3.36E+02 | 8.88E−04 | 3.67E−05 | 1.5971 | 0.9961 |
| SLYNTVATE | 6.96E−06 | 9.43E−06 | 3.79E+02 | 5.13E+02 | 2.64E−03 | 8.02E−05 | 0.1997 | 0.9908 |
| SLYNTVATD | 7.18E−06 | 8.48E−06 | 3.95E+02 | 4.67E+02 | 2.83E−03 | 7.30E−05 | 0.1137 | 0.9924 |
| SLYNTVATS | 7.19E−09 | 2.13E−10 | 1.16E+05 | 2.54E+02 | 8.33E−04 | 2.46E−05 | 0.7637 | 0.9981 |
| SLYNTVATT | 5.66E−09 | 1.27E−10 | 1.12E+05 | 1.42E+02 | 6.32E−04 | 1.41E−05 | 0.2627 | 0.9994 |

TABLE 4
Cross-reactive peptide ligand search motif for
bs-868Z11-CD3 based on the affinities measured
using the positional scanning library. All amino
acids of the 19 proteinogenic amino acids
investigated at each position that increased the
respective affinity of the bsTCR above 50 nM
were removed to reach the search motif.

| Peptide | Permitted |
|---|---|
| Position | Amino Acids |
| 1 | GPAVLIMKRNDST |
| 2 | GAVLIMFYHQNEST |
| 3 | FYW |
| 4 | N |
| 5 | VLIMT |
| 6 | PVLIMT |
| 7 | GPANS |
| 8 | GAVLIMHKRQNEDST |
| 9 | GAVLIMFYWHKRQNST |

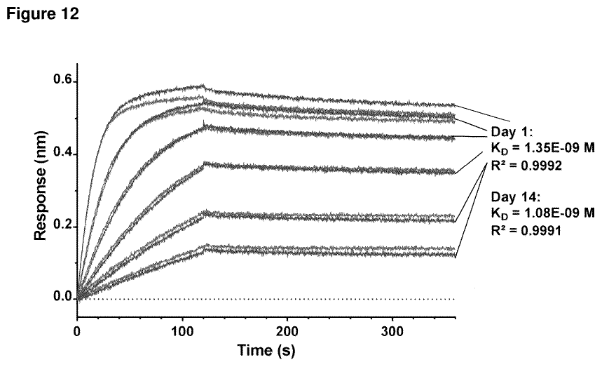

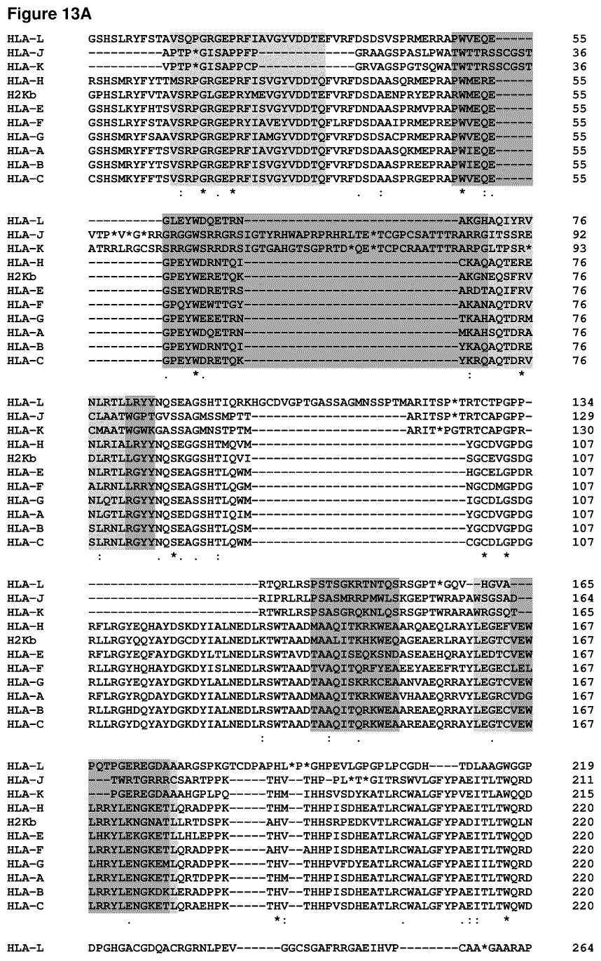

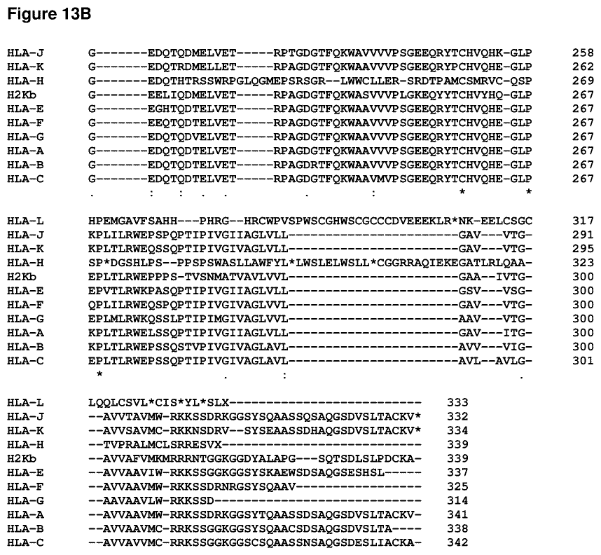

Soluble Y84C/A139C HLA-A*02:01 pMHC preparations can be stored for at least 2 weeks at 4° C. without loss of quality and used for multiple analyses (FIG. 12; Day 1: K_{D}=1.35E⁻⁰⁹ M, R²=0.9992; Day 14: K_{D}=1.08E⁻⁰⁹ M, R²=0.9991).

The 868Z11 TCR displayed an expected pattern of recognition: changes of amino acids between positions 3 to 7 had the biggest influence on the bsTCR binding affinity. Interestingly, only one amino acid change resulted in an increased binding affinity by bs-868Z11-CD3 compared to the interaction with the wild type peptide, showcasing the remarkable affinity the TCR has for the target in its affinity maturated state. This behavior can also be graphically illustrated when visualizing the binding motif as Seq2Logo graph (FIG. 4B) (27).

21. Identification of Peptide Ligands Cross-Reactive with bs-868Z11-CD3

The inventors further wanted to explore whether they could use the generated binding motif to identify cross-reactive peptide ligands from the human genome. The inventors created a peptide ligand search motif from the affinity dataset by introducing an exemplary K_{d} threshold of 50 nM: all single amino acid substitutions increasing the bs-868Z11-CD3 K_{d} above that threshold were excluded from the motif (Table 4). Based on this motif the inventors performed a search in the NCBI human non-redundant protein sequence database for nonamer sequences matching combinations allowed by the motif. The search identified over 400 hits within the human genome, with sequence identity to the wild type sequence SLYNTVATL ranging from 1 to 6 identical positions. 140 peptides were selected, sampled to be representative of the sequence identity distribution in the larger group, synthesized and used for affinity measurements (Table 5; SEQ ID NOS: 178-317). The inventors were able to detect binding affinities of single digit μM K_{d}s or higher for 91 of those peptides.

TABLE 5
bs-868Z11-CD3 binding affinity for selected peptide ligands identified based
on the bz-868Z11-CD3 binding motif.
Peptide sequences and associated genes according
to the NCBI data base are reported and peptides are sorted by decreasing K_{d}s. Table
includes K_{D}, kₒₙ, and k_{off} values determined by curve fittings following a 1:1 Langmuir
binding model using the Fortébio Data Analysis HT 10.0.3.7 software. Respective errors
are reported as well as accuracy of the fit according to the model. Peptides reported as
“No fit” had no evaluable curves reaching at least a peak signal of 0.05 nm at any
concentration.

| | Associated | | | kₒₙ | kₒₙ | k_{off} | k_{off} | Full | Full |
|---|---|---|---|---|---|---|---|---|---|
| Peptide | Gene | KD (M) | KD Error | (M⁻¹s⁻¹) | Error | (s⁻¹) | Error | X² | R² |
| RVYNTVPLV | HIPK3 | 1.32E−08 | 1.76E−10 | 9.50E+04 | 1.69E+02 | 1.26E−03 | 1.66E−05 | 0.2908 | 0.9993 |
| RMYNLVSRI | CUL1 | 1.91E−08 | 2.52E−10 | 9.25E+04 | 2.28E+02 | 1.76E−03 | 2.29E−05 | 0.5429 | 0.9986 |
| SLYNMVPSI | OVOS | 1.97E−08 | 1.71E−10 | 1.31E+05 | 2.89E+02 | 2.57E−03 | 2.16E−05 | 0.586 | 0.9987 |
| TVYNMVPSI | OVOS | 2.07E−08 | 1.54E−10 | 1.28E+05 | 2.50E+02 | 2.66E−03 | 1.91E−05 | 0.4228 | 0.999 |
| ALYNVIAMA | SECISBP2L | 2.11E−08 | 1.51E−10 | 9.28E+04 | 2.25E+02 | 1.96E−03 | 1.32E−05 | 0.114 | 0.9997 |
| AIYNLLPDI | NCAPD2 | 2.33E−08 | 1.82E−10 | 1.01E+05 | 1.90E+02 | 2.36E−03 | 1.79E−05 | 0.3299 | 0.9992 |
| STYNLVSTS | KIAA2018 | 2.47E−08 | 2.16E−10 | 7.07E+04 | 1.28E+02 | 1.75E−03 | 1.49E−05 | 0.1772 | 0.9995 |
| SVYNMVPSI | OVOS2 | 2.68E−08 | 1.96E−10 | 1.32E+05 | 3.26E+02 | 3.53E−03 | 2.43E−05 | 0.6471 | 0.9984 |
| RTYNVLAIL | ATP8B1 | 3.11E−08 | 1.55E−10 | 7.54E+04 | 9.93E+01 | 2.34E−03 | 1.12E−05 | 0.0937 | 0.9997 |
| SVYNLVSIA | KPTN | 3.65E−08 | 2.01E−10 | 7.97E+04 | 2.13E+02 | 2.91E−03 | 1.40E−05 | 0.0926 | 0.9997 |
| RAYNLIGTV | LOC100128501 | 3.72E−08 | 1.71E−10 | 8.94E+04 | 1.40E+02 | 3.33E−03 | 1.43E−05 | 0.1721 | 0.9995 |
| ALFNLIPVG | FGF12 | 3.83E−08 | 3.22E−10 | 6.64E+04 | 1.71E+02 | 2.54E−03 | 2.04E−05 | 0.266 | 0.999 |
| RIYNVIGTL | FOLH1, FOLH1B | 4.53E−08 | 3.00E−10 | 5.75E+04 | 1.28E+02 | 2.61E−03 | 1.62E−05 | 0.1095 | 0.9994 |
| RIYNVVGTI | NAALAD2 | 5.15E−08 | 4.13E−10 | 5.94E+04 | 1.81E+02 | 3.06E−03 | 2.27E−05 | 0.2723 | 0.9989 |
| TLFNLVPNS | CLASP2 | 5.40E−08 | 3.31E−10 | 9.76E+04 | 2.90E+02 | 5.28E−03 | 2.82E−05 | 0.4822 | 0.9981 |
| SLFNVISIL | KCNK12, KCNK13 | 5.83E−08 | 3.11E−10 | 6.94E+04 | 1.64E+02 | 4.05E−03 | 1.94E−05 | 0.2068 | 0.9992 |
| STFNLVAIS | CCKAR | 6.06E−08 | 2.91E−10 | 4.96E+04 | 9.98E+01 | 3.01E−03 | 1.31E−05 | 0.0436 | 0.9996 |
| TLFNLIPVG | FGF12, FGF13, | 6.32E−08 | 3.97E−10 | 6.72E+04 | 1.98E+02 | 4.25E−03 | 2.36E−05 | 0.3044 | 0.9988 |
| | FGF14 | | | | | | | | |
| TIFNLIPNS | CLASP1 | 6.41E−08 | 2.67E−10 | 8.97E+04 | 1.99E+02 | 5.75E−03 | 2.03E−05 | 0.2655 | 0.999 |
| ALYNVLAKV | IFFO1, IFFO2 | 6.59E−08 | 2.96E−10 | 1.02E+05 | 2.63E+02 | 6.75E−03 | 2.49E−05 | 0.3602 | 0.9986 |
| AVFNLLPHT | SMYD4 | 7.11E−08 | 2.72E−10 | 8.53E+04 | 1.82E+02 | 6.07E−03 | 1.93E−05 | 0.2344 | 0.9991 |
| RMYNLLGHM | ZNF710 | 8.71E−08 | 7.55E−10 | 5.30E+04 | 2.55E+02 | 4.62E−03 | 3.33E−05 | 0.2278 | 0.9977 |
| STWNTPPNM | KIAA0922 | 8.98E−08 | 3.45E−10 | 9.05E+04 | 2.28E+02 | 8.13E−03 | 2.36E−05 | 0.2591 | 0.9989 |
| NIYNLIAII | BICD2 | 9.32E−08 | 3.91E−10 | 1.06E+05 | 3.10E+02 | 9.84E−03 | 2.95E−05 | 0.3894 | 0.9983 |
| RIYNLPPEL | WRAP53 | 9.95E−08 | 3.78E−10 | 9.47E+04 | 2.51E+02 | 9.42E−03 | 2.56E−05 | 0.3026 | 0.9988 |
| TTFNLPSAA | WDR17 | 1.02E−07 | 6.71E−10 | 7.87E+04 | 3.47E+02 | 8.04E−03 | 3.91E−05 | 0.579 | 0.997 |
| MFFNVIAIV | UGGT2 | 1.06E−07 | 1.29E−09 | 5.21E+04 | 3.95E+02 | 5.52E−03 | 5.23E−05 | 0.0772 | 0.9934 |
| SLWNTVSGI | HHLA1 | 1.08E−07 | 5.04E−10 | 8.88E+04 | 2.94E+02 | 9.60E−03 | 3.14E−05 | 0.3873 | 0.9982 |
| MLWNLLALR | COX7A2 | 1.17E−07 | 1.03E−08 | 1.26E+06 | 1.05E+05 | 1.47E−01 | 4.25E−03 | 0.0448 | 0.9675 |
| VFWNLLPTV | C12orf74 | 1.20E−07 | 7.20E−10 | 1.24E+05 | 5.97E+02 | 1.49E−02 | 5.29E−05 | 0.9116 | 0.9955 |
| STFNTTSNG | QSER1 | 1.52E−07 | 6.60E−09 | 1.76E+05 | 7.05E+03 | 2.67E−02 | 4.39E−04 | 0.0311 | 0.9225 |
| GFFNLLSHV | PCP2 | 1.59E−07 | 9.88E−09 | 6.24E+05 | 3.64E+04 | 9.94E−02 | 2.08E−03 | 0.0321 | 0.9717 |
| LLYNVPAVA | APP | 1.67E−07 | 1.27E−08 | 7.41E+05 | 5.29E+04 | 1.24E−01 | 3.21E−03 | 0.0164 | 0.9712 |
| ALFNTISQG | VTA1 | 1.83E−07 | 8.26E−10 | 7.15E+04 | 2.68E+02 | 1.31E−02 | 3.28E−05 | 0.2214 | 0.9984 |
| TTFNTLAGS | MUC16 | 1.97E−07 | 8.70E−10 | 9.03E+04 | 3.44E+02 | 1.78E−02 | 3.98E−05 | 0.1556 | 0.9981 |
| SLWNLLGNA | LMAN2L | 2.14E−07 | 3.02E−08 | 1.02E+06 | 1.35E+05 | 2.19E−01 | 1.07E−02 | 0.0324 | 0.952 |
| SLYNLLNLT | SLC4A5 | 2.19E−07 | 8.40E−10 | 6.75E+04 | 2.24E+02 | 1.48E−02 | 2.85E−05 | 0.094 | 0.9988 |
| GVWNLLSIV | ZSWIM8 | 2.52E−07 | 4.59E−08 | 1.09E+06 | 1.86E+05 | 2.74E−01 | 1.73E−02 | 0.0545 | 0.9403 |
| ALFNVVNSI | SLC38A11 | 2.55E−07 | 2.10E−09 | 6.83E+04 | 4.99E+02 | 1.74E−02 | 6.59E−05 | 0.2897 | 0.9956 |
| VIYNLLGLA | SH3TC2 | 2.64E−07 | 1.88E−09 | 1.07E+05 | 7.25E+02 | 2.83E−02 | 5.92E−05 | 0.2115 | 0.9983 |
| SIFNIVAIA | GPR50 | 2.84E−07 | 1.60E−09 | 4.44E+04 | 2.37E+02 | 1.26E−02 | 2.25E−05 | 0.0369 | 0.9995 |
| TVYNTVSEG | SLC39A6 | 3.04E−07 | 2.50E−09 | 4.71E+04 | 3.50E+02 | 1.43E−02 | 4.99E−05 | 0.2538 | 0.997 |
| DLWNTLSSL | EFCAB13, ITGB3 | 3.39E−07 | 2.60E−08 | 4.15E+05 | 3.02E+04 | 1.41E−01 | 3.55E−03 | 0.0238 | 0.9752 |
| IFFNLLAVL | POMT1 | 3.50E−07 | 4.75E−08 | 8.47E+05 | 1.08E+05 | 2.97E−01 | 1.35E−02 | 0.023 | 0.9688 |
| DLFNLLPDV | PSMD7 | 3.60E−07 | 1.08E−08 | 7.69E+04 | 2.15E+03 | 2.77E−02 | 3.14E−04 | 0.0881 | 0.9367 |
| LSWNVVPNA | SPCS3 | 3.67E−07 | 2.91E−08 | 4.13E+05 | 3.09E+04 | 1.52E−01 | 3.89E−03 | 0.0234 | 0.9734 |
| MLWNLLALH | COX7A2P2 | 3.67E−07 | 2.04E−08 | 1.07E+06 | 4.75E+04 | 3.94E−01 | 1.33E−02 | 0.1159 | 0.9595 |
| TIFNTVNTS | TIMMDC1 | 3.87E−07 | 2.77E−09 | 4.20E+04 | 2.80E+02 | 1.63E−02 | 4.19E−05 | 0.0304 | 0.9978 |
| KTFNLIPAV | MRPL4 | 4.13E−07 | 2.59E−09 | 1.12E+05 | 6.52E+02 | 4.62E−02 | 1.07E−04 | 0.1185 | 0.9979 |
| NLFNVTPLI | ZNF66P | 4.28E−07 | 1.38E−07 | 1.05E+06 | 3.19E+05 | 4.49E−01 | 4.68E−02 | 0.0447 | 0.9139 |
| SYWNIISTV | OR2D3 | 4.39E−07 | 4.84E−09 | 4.56E+04 | 4.74E+02 | 2.00E−02 | 7.23E−05 | 0.1373 | 0.9952 |
| GVENLIAVL | AC002365.5, | 4.59E−07 | 4.94E−09 | 7.59E+04 | 7.70E+02 | 3.48E−02 | 1.26E−04 | 0.3268 | 0.9946 |
| | LOC100288814 | | | | | | | | |
| RLFNITSSA | IFITM10 | 4.74E−07 | 4.12E−08 | 2.51E+05 | 2.08E+04 | 1.19E−01 | 3.08E−03 | 0.0167 | 0.9706 |
| NLWNLVAVI | WDR17 | 4.97E−07 | 1.16E−08 | 2.07E+05 | 4.34E+03 | 1.03E−01 | 1.04E−03 | 0.256 | 0.9836 |
| RIFNLIGMM | HCN1, HCN3 | 4.98E−07 | 1.26E−08 | 2.29E+04 | 5.51E+02 | 1.14E−02 | 8.55E−05 | 0.0712 | 0.9889 |
| RLFNVVSRG | TRPV2 | 5.02E−07 | 6.55E−09 | 6.50E+04 | 8.05E+02 | 3.26E−02 | 1.34E−04 | 0.1875 | 0.9931 |
| LVFNVIPTL | ABCB6 | 5.35E−07 | 3.99E−09 | 1.33E+05 | 9.21E+02 | 7.13E−02 | 2.00E−04 | 0.0445 | 0.9982 |
| TTWNILSSA | COX1 | 5.36E−07 | 4.08E−08 | 2.26E+05 | 1.65E+04 | 1.21E−01 | 2.63E−03 | 0.0214 | 0.9794 |
| KLFNVLSTL | NUP210P2 | 5.76E−07 | 3.35E−08 | 2.97E+05 | 1.65E+04 | 1.71E−01 | 2.83E−03 | 0.0131 | 0.9912 |
| RVYNLTAKS | VWA3B | 5.95E−07 | 4.57E−09 | 4.53E+04 | 3.35E+02 | 2.69E−02 | 5.65E−05 | 0.0219 | 0.9981 |
| LTFNTISLS | ENTHD1 | 7.09E−07 | 2.12E−07 | 4.78E+05 | 1.37E+05 | 3.39E−01 | 2.72E−02 | 0.0387 | 0.929 |
| AQFNLLSST | TP73 | 7.13E−07 | 9.97E−09 | 8.59E+04 | 1.15E+03 | 6.12E−02 | 2.58E−04 | 0.1658 | 0.9947 |
| VVYNVLSEL | SP100, SP140L | 7.35E−07 | 6.29E−08 | 1.84E+05 | 1.52E+04 | 1.35E−01 | 2.89E−03 | 0.0255 | 0.9785 |
| KVYNTPSTS | AEBP2 | 7.51E−07 | 1.39E−08 | 1.71E+05 | 2.91E+03 | 1.28E−01 | 9.13E−04 | 0.0718 | 0.9945 |
| GIFNIIPST | CAPN7 | 7.90E−07 | 8.67E−09 | 1.32E+05 | 1.36E+03 | 1.04E−01 | 4.00E−04 | 0.0364 | 0.9979 |
| NIYNTLSGL | UBR4 | 8.73E−07 | 1.71E−08 | 1.59E+05 | 2.89E+03 | 1.38E−01 | 9.89E−04 | 0.0564 | 0.9947 |
| RLFNLTSTF | FLJ44715, FUT11 | 9.32E−07 | 2.82E−08 | 1.72E+05 | 4.83E+03 | 1.60E−01 | 1.79E−03 | 0.0525 | 0.9894 |
| TVWNTLSSL | DNAH9 | 9.39E−07 | 1.22E−08 | 4.52E+04 | 5.73E+02 | 4.25E−02 | 1.20E−04 | 0.0344 | 0.9971 |
| RLFNMLSAV | CFAP221, PCDP1 | 9.71E−07 | 3.06E−08 | 1.73E+05 | 5.09E+03 | 1.68E−01 | 1.94E−03 | 0.0714 | 0.9892 |
| SIWNVTAIA | HTR5A | 1.10E−06 | 5.12E−07 | 3.21E+05 | 1.45E+05 | 3.54E−01 | 3.47E−02 | 0.0576 | 0.9051 |
| ALFNLMSGI | EGR4 | 1.19E−06 | 3.21E−08 | 9.57E+04 | 2.48E+03 | 1.14E−01 | 8.53E−04 | 0.0645 | 0.9931 |
| IVYNLLSAM | SLC39A10 | 1.30E−06 | 1.62E−07 | 1.61E+05 | 1.98E+04 | 2.10E−01 | 4.82E−03 | 0.0245 | 0.987 |
| ISFNMLPSI | GPR98 | 1.37E−06 | 4.65E−08 | 1.24E+05 | 4.04E+03 | 1.70E−01 | 1.70E−03 | 0.0581 | 0.991 |
| NTYNILPGS | C9orf173 | 1.38E−06 | 1.17E−07 | 1.14E+05 | 9.57E+03 | 1.57E−01 | 2.30E−03 | 0.025 | 0.9925 |
| RLWNMVNVT | IL12RB2 | 1.39E−06 | 2.57E−07 | 1.52E+05 | 2.77E+04 | 2.11E−01 | 6.87E−03 | 0.049 | 0.9763 |
| SAFNITSLI | WAC | 1.41E−06 | 3.21E−07 | 1.65E+05 | 3.70E+04 | 2.32E−01 | 9.29E−03 | 0.0314 | 0.9682 |
| NIFNLPNIV | OMD | 1.48E−06 | 6.62E−07 | 4.19E+05 | 1.85E+05 | 6.20E−01 | 4.90E−02 | 0.0905 | 0.9596 |
| GVYNLPGAS | GPX2 | 1.58E−06 | 3.07E−07 | 1.17E+05 | 2.23E+04 | 1.84E−01 | 5.65E−03 | 0.0488 | 0.9756 |
| GTYNVISLV | TRPC4, TRPC5 | 1.64E−06 | 4.18E−07 | 1.23E+05 | 3.10E+04 | 2.02E−01 | 8.00E−03 | 0.0666 | 0.965 |
| SIFNTLSDI | SGSM3 | 1.97E−06 | 5.86E−08 | 4.07E+04 | 1.20E+03 | 8.01E−02 | 3.78E−04 | 0.0856 | 0.9957 |
| TIFNILSGI | ABCA3 | 2.66E−06 | 2.37E−07 | 4.68E+04 | 4.13E+03 | 1.24E−01 | 1.66E−03 | 0.1728 | 0.9807 |
| LLFNLISSS | MON1A | 2.79E−06 | 2.57E−06 | 1.51E+05 | 1.38E+05 | 4.20E−01 | 4.10E−02 | 0.0599 | 0.9183 |
| RTFNLTAGS | PDXDC1 | 2.85E−06 | 5.89E−07 | 4.63E+04 | 9.54E+03 | 1.32E−01 | 2.56E−03 | 0.0356 | 0.9845 |
| TVFNILPGG | PAFAH2 | 3.23E−06 | 1.06E−06 | 3.69E+04 | 1.21E+04 | 1.19E−01 | 3.22E−03 | 0.025 | 0.968 |
| GLFNIPPAS | CYP2S1 | 3.91E−06 | 4.13E−06 | 8.52E+04 | 8.97E+04 | 3.33E−01 | 2.74E−02 | 0.0395 | 0.9216 |
| RMFNIISDS | RASA1 | 3.99E−06 | 4.08E−07 | 1.51E+04 | 1.54E+03 | 6.02E−02 | 4.39E−04 | 0.0509 | 0.9862 |
| TTFNIVGTT | GABRA3 | 6.79E−06 | 3.10E−06 | 8.68E+03 | 3.96E+03 | 5.89E−02 | 8.72E−04 | 0.0351 | 0.9743 |
| ALFNLMSGV | EGR4 | 7.87E−06 | 1.14E−05 | 3.17E+04 | 4.60E+04 | 2.50E−01 | 1.45E−02 | 0.1115 | 0.9454 |
| SVFNITAIA | MTNR1B | 1.96E−05 | 2.39E−04 | 2.58E+04 | 3.15E+05 | 5.06E−01 | 1.06E−01 | 0.2805 | 0.7869 |
| KIYNTPSAS | NCAM1 | 2.56E−05 | 2.88E−05 | 8.86E+03 | 9.95E+03 | 2.27E−01 | 5.62E−03 | 0.2474 | 0.9662 |
| LLYNLLGSS | ABCC9 | 1.41E−04 | 6.82E−03 | 3.21E+03 | 1.55E+05 | 4.54E−01 | 5.27E−02 | 0.1153 | 0.9007 |
| SLYNMMGEA | TMTC2 | No fit | | | | | | | |
| SLWNLMGNA | LMAN2L | No fit | | | | | | | |
| GLYNIVGNA | SUMF1 | No fit | | | | | | | |
| LTWNLTPKA | DLEC1 | No fit | | | | | | | |
| LIFNVTGLA | ZDHHC23 | No fit | | | | | | | |
| SIFNITGIA | MTNR1A | No fit | | | | | | | |
| LTFNLVSDA | CASP8AP2 | No fit | | | | | | | |
| MQWNILAQA | CCRN4L | No fit | | | | | | | |
| LSWNLVPEA | COL7A1 | No fit | | | | | | | |
| DLWNTLSEA | TRHDE | No fit | | | | | | | |
| GLFNIPPAF | CYP2S1 | No fit | | | | | | | |
| LIWNILASF | TTC29 | No fit | | | | | | | |
| LLFNMLPGG | EXT2 | No fit | | | | | | | |
| LVYNIMSSG | FAM120B | No fit | | | | | | | |
| IIYNVPGTG | RNF133 | No fit | | | | | | | |
| VIYNVTSDG | TTN | No fit | | | | | | | |
| GTFNLPSDG | BAG6 | No fit | | | | | | | |
| KLWNTLNLI | ENPP5 | No fit | | | | | | | |
| LMWNIISII | VTCN1 | No fit | | | | | | | |
| GLFNTTSNI | SEMA3E | No fit | | | | | | | |
| LIFNTLSLI | PDCD6IP | No fit | | | | | | | |
| SVFNLMNAI | SLC38A6 | No fit | | | | | | | |
| LTFNILGQI | DOCK11 | No fit | | | | | | | |
| GLFNMVSSL | RRN3 | No fit | | | | | | | |
| KIFNIINSL | FER1L5 | No fit | | | | | | | |
| AVWNVLGNL | BAGS | No fit | | | | | | | |
| KVFNIVSDL | FSIP2 | No fit | | | | | | | |
| DLWNVVSHL | DDX60L | No fit | | | | | | | |
| LQFNTVSKL | JAM2 | No fit | | | | | | | |
| MSFNTVSEL | ZNF33A, ZNF33B | No fit | | | | | | | |
| ASWNIVNLL | TRPA1 | No fit | | | | | | | |
| ISFNIISAL | MS4A18 | No fit | | | | | | | |
| AFFNILNEL | FNBP1L | No fit | | | | | | | |
| LVFNLLPIM | ABCB7 | No fit | | | | | | | |
| KIFNTVPDM | ARHGAP26 | No fit | | | | | | | |
| MLFNLIGLS | OR10J1 | No fit | | | | | | | |
| LLFNLPPGS | VGLL1 | No fit | | | | | | | |
| MTFNLIGES | CR1, CR1L | No fit | | | | | | | |
| KVYNIPGIS | KLI1L10 | No fit | | | | | | | |
| GIYNIPGDS | TNS1 | No fit | | | | | | | |
| GLYNLMNIT | INSR | No fit | | | | | | | |
| LTWNMINTT | LRIT3 | No fit | | | | | | | |
| IVFNVLSDT | HCN3 | No fit | | | | | | | |
| IVFNVVSDT | HCN2, HCN4 | No fit | | | | | | | |
| LIFNITASV | SVEP1 | No fit | | | | | | | |
| IVFNLTNNV | MNAT1 | No fit | | | | | | | |
| KSFNVLSSV | ZNF557 | No fit | | | | | | | |
| LAFNILGMV | SLC46A1 | No fit | | | | | | | |
| VSWNITGTV | SEH1L | No fit | | | | | | | |

One of them, ALYNVLAKV (SEQ ID NO: 1), was worth of special notice. It was selected as a theoretical peptide but found in addition on tissue samples and cell lines according to the XPRESIDENT® immunopeptidomics database. This database combines quantitative HLA peptidomics based on LC-MS analysis and quantitative transcriptomics provided by RNAseq from healthy tissues and tumor tissues to identify peptides presented exclusively or predominately on tumor tissue (28, 29). ALYNVLAKV, an antigen from intermediate filament family orphan 1 or 2 (IFFO1/2), was detected on multiple healthy tissue and tumor tissue samples, ranging from head and neck, spleen, or kidney to non-small cell lung carcinoma or renal cell carcinoma. The pMHC-bsTCR binding affinity was measured with a K_{D} of 65.9 nM (FIG. 4c). The inventors were able to identify a second LC-MS detected peptide, KTFNLIPAV (SEQ ID NO: 226), with a lower K_{d} of 413 nM detected on three tumor tissue samples.

22. Correlation of bsTCR Affinity with T cell Activation

The pMHC-bsTCR binding affinity can be measured using this high-throughput screening platform, but should be consistent with the in vitro activity as functional T cell engaging bsTCR to be even more useful. Commonly, in vitro co-incubations of target and effector cells coupled with an appropriate readout are used to characterize these constructs. GloResponse™ NFAT-luc2 Jurkat effector cells, a cell line that expresses a luciferase reporter gene driven by a NFAT-response element, and peptide-loaded T2 target cells, a TAP-deficient A*02:01 cell line with restorable pMHC presentation through exogenous peptide loading, were incubated in the presence of bs-868Z11-CD3 to corroborate the significance of the kinetic screening in this context. T2 cells were loaded separately with respective peptides from the positional scanning library at a concentration of 100 nM and subsequently co-incubated with Jurkats and different bsTCR concentrations for 18 hours before readout. As expected the inventors encountered a broad spectrum of results, ranging from no detectable T cell activation at any bsTCR concentration to strong responses starting at low concentrations, e.g. for the wild type peptide (FIG. 5A). Since EC50 values could not be determined for many of the interactions in the selected bsTCR concentration range the inventors categorized the individual peptides by onset of T cell activation, defined as the lowest bsTCR concentration that was able to induce a 3-fold increased signal above. Onset values were plotted against the respectively measured K_{D}s (FIG. 5B).

Overall, the inventors detected a good correlation between the determined K_{d} values and T cell activation with one notable group of outliers with strong pMHC-bsTCR binding affinities but late T cell activation onset or no activation at all. The inventors were able to identify a direct connection between these peptides and their NetMHC predicted binding strength to the MHC (FIG. 5C) (26). This offered a potential explanation because different peptide binding affinities could result in different presentation levels of the respective pMHCs on the target cells after exogenous loading. These levels might, in turn, influence pMHC-bsTCR complex numbers and ultimately Jurkat effector (T cell) activation. To corroborate the hypothesis, the inventors performed a flow cytometric T2 peptide binding assay using an anti-HLA-A2 antibody and could detect less elevated HLA-A2 surface levels after peptide loading for peptides with lower binding affinities, especially NetMHC ranks of 2 and above, supporting the initial hypothesis. pMHC-bsTCR binding affinity correlated well with T cell activation onset for peptide ligands between NetMHC rank 0.05 and 2, whereas above that threshold T cell activation decreased with further increasing NetMHC ranks largely irrespective of pMHC-bsTCR binding affinity.

The inventors also performed T cell activation assays for the 140 peptide ligands selected by binding motif search, 24 were capable of inducing a 3-fold T cell activation over background with at least one of the supplied bsTCR concentrations (FIG. 5D). Measured K_{d}s correlated with the onset of T cell activation similarly to the results obtained by the positional scanning library. The previously highlighted IFFO1 antigen ALYNVLAKV (SEQ ID NO: 1) was also reactive in the reporter assay (FIG. 5E).

The inventors showed that pMHC-bsTCR binding affinity is a good indicator for the in vitro function of the scTv 868Z11 coupled with an anti-CD3 T cell engager. This highlights the value of the pMHC-bsTCR binding kinetics screening platform because it allows quick but adequate characterization of bsTCRs early in the development of such molecules.

### 23. Crystal Structure of the 1G4 Y84C/A139C HLA-A*02:01:01 ESO 9V TCR-pMHC

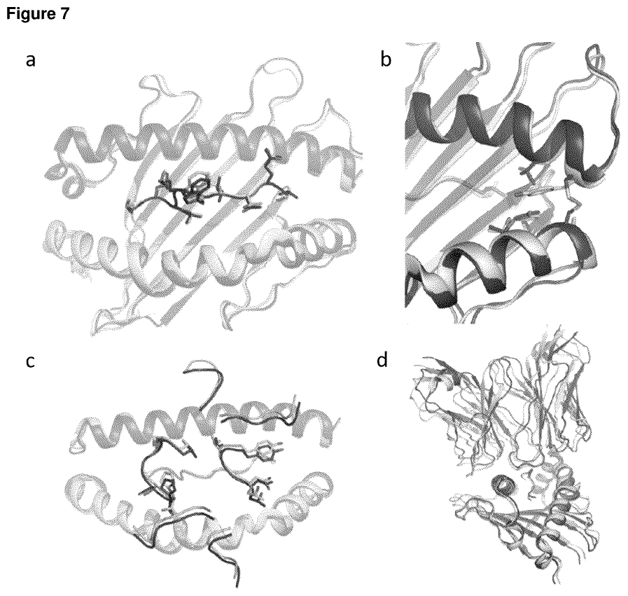

To further confirm that the 1G4 TCR recognizes ESO 9VY84C/A139C HLA-A*02:01 indistinguishably from ESO 9V WT-A*02:01. TCR and disulfide-stabilized MHC refolded with ESO 9V were cocrystallized, as reported previously for the wild-type ESO 9V HLA-A*02:01 molecule and analyzed by x-ray crystallography (Table 2) (21). Comparison of the crystal structures revealed a high degree of structural overlap between both complexes. The backbone of both HLA-A*02:01 molecules aligned almost perfectly with a root mean square deviation (RMSD) value of 1.14 Å calculated over Cα (constant portion of the a chain of a T cell receptor; FIG. 7A). The same was true for both bound peptides including their side chains with an RMSD value of 1.27 Å calculated over all atoms, even when in close vicinity to the disulfide bond (FIG. 7B). Similar conclusions could be made for the interaction with the 1G4 TCR. The complementarity-determining region (CDR) loop regions interacting with the peptide and the MHC backbone did show slight deviations of the interface and a small change in the docking angle of 4.13°, when comparing WT-A*02:01 1G4 with the Y84C/A139C HLA-A*02:01 1G4 crystal structure. This shift was still within the range of expected deviations for the same complex when crystallized repeatedly (FIG. 7, C and D). Together, determined binding affinities and crystal structure showcase peptide receptiveness and similar properties of the Y84C/A139C HLA-A*02:01 pMHC complexes compared with wild-type complexes with respect to TCR binding. The crystal structure of the 1G4 Y84C/A139C HLA-A*02:01 ESO 9V complex has been deposited in the PDB under the accession number 6Q3S.

### 24. Discussion

Here, the inventors have presented disulfide-stabilized and functionally empty HLA-A*02:01 molecules, which can be refolded and purified without the use of typically required high-affinity peptides e.g. the dipeptide GM. The resulting monomers can form pMHCs after addition of peptides in a one-step loading procedure. Although the disulfide bridge enhances the stability of the MHC molecule, introduction does not inhibit or significantly alter binding of TCRs to disulfide-modified HLA*02:01 pMHC complexes compared with the wild type. This technique represents a great tool to quickly produce large pMHC libraries that are suitable for affinity measurements. Combining disulfide modified HLA*02:01-produced pMHC complex libraries with biolayer interferometry-based analysis results in a platform capable of high-throughput pMHC-bsTCR binding kinetics screenings. This setup could also be useful for the analysis of other biologics targeting pMHC complexes, like monoclonal antibodies or bispecifics, such as bispecific T cell engagers. In one application of this platform, the inventors were able to quickly collect a pMHC-bsTCR binding affinity dataset for the HIV-specific bsTCR bs-868Z11-CD3. bsTCR binding affinities for respective pMHCs were indicative of in vitro activity when coupled with the presented T cell engager and tested in a cellular reporter assay, making these datasets valuable for bsTCR characterization. Analysis of the relationship between binding affinity and bsTCR-mediated cellular activation over a wide range of pMHC-bsTCR affinities has been difficult, thus far as a result of the limited tools available to feasibly collect such datasets.

The collected binding motif revealed similarities to the binding motif of the wild-type TCR 868. Analysis of an 868-SV9 crystal structure, as well as an accompanying alanine scan by Cole et al. (34), revealed prominent interactions between the CDR3α region and the amino acids 4N and 5T of SLYNTVATL. This behavior seems to be conserved although a significant part of the CDR3α is mutated in the 868Z11 construct. Using the binding motif and a model search strategy, the inventors were able to identify multiple peptides from the human proteome, which demonstrated high-affinity interactions with the bsTCR and the potential to induce bsTCR-mediated Jurkat effector activation when presented on target cells.

Note that TCR binding motifs derived from single amino acid substitution libraries may still not reflect all possible peptides a specific TCR (sTCR) can recognize, because the exchange of multiple amino acids, at the same time, might have different effects than the isolated exchanges. Alternative approaches include screening of more complex libraries, for example, through target cell loading with high diversity peptide pools, each randomized at all but one position of the peptide, or screenings against randomized peptide libraries presented as pMHC complexes on yeast surfaces (10, 32, 33). Further research directly comparing these approaches will be necessary to gain a deeper understanding of the respective strengths and weaknesses. Ultimately, safety screenings of clinical candidates should always be composed of multiple approaches, for example, by combining binding motif guided analysis together with cellular screenings of large panels of healthy tissue-derived cell lines, to minimizing risks. The results presented herein highlight the capability of this approach to identifying potentially relevant off-target interactions in combination with the pMHC-bsTCR binding kinetics screening platform. Because it offers quick analysis of complex pMHC libraries, it can be used early in the development process to select promising candidates and thus, complements established methods. This platform can also facilitate larger and more comprehensive screenings of late-stage candidates, potentially against mass spectrometry data-driven tissue-specific pMHC libraries covering the known immunopeptidome. Because of its stability and low-effort peptide loading procedure, the disulfide-modified HLA*02:01 molecules could potentially enable even higher-throughput platforms. Thanks to these properties, it could be perfectly suited for the creation of high complexity pMHC microarrays with thousands of different pMHC complexes, for example, by combining large-scale coating of disulfide-modified HLA*02:01 molecules and modern high-throughput peptide microarray inkjet printers.

### References as cited

1. Rammensee, H. G., Falk, K. & Rotzschke, O. Peptides naturally presented by MHC class I molecules. Annu. Rev. Immunol. 11, 213-44 (1993).
2. Garboczi, D. N. et al. HLA-A2-peptide complexes: refolding and crystallization of molecules expressed in Escherichia coli and complexed with single antigenic peptides. Proc. Natl. Acad. Sci. U.S.A. 89, 3429-3433 (1992).
3. Altman, J. D. et al. Phenotypic analysis of antigen-specific T lymphocytes. Science (80-.). 274, 94-96 (1996).
4. Hadrup, S. R. et al. High-throughput T-cell epitope discovery through MHC peptide exchange. Methods Mol. Biol. 524, 383-405 (2009).
5. Garcia, K. C. et al. Alphabeta T cell receptor interactions with syngeneic and allogeneic ligands: affinity measurements and crystallization. Proc. Natl. Acad. Sci. U.S.A. 94, 13838-43 (1997).
6. Stone, J. D. & Kranz, D. M. Role of T Cell Receptor Affinity in the Efficacy and Specificity of Adoptive T Cell Therapies. Front. Immunol. 4, 1-16 (2013).
7. Robbins, P. F. et al. Single and dual amino acid substitutions in TCR CDRs can enhance antigen-specific T cell functions. J. Immunol. 180, 6116-6131 (2008).
8. Varela-Rohena, A. et al. Control of HIV-1 immune escape by CD8 T cells expressing enhanced T-cell receptor. Nat. Med. 14, 1390-1395 (2008).
9. Oates, J., Hassan, N. J. & Jakobsen, B. K. ImmTACs for targeted cancer therapy: Why, what, how, and which. Mol. Immunol. 67, 67-74 (2015).
10. Wooldridge, L. et al. A single autoimmune T cell receptor recognizes more than a million different peptides. J. Biol. Chem. 287, 1168-1177 (2012).
11. Cameron, B. J. et al. Identification of a Titin-derived HLA-A1-presented peptide as a cross-reactive target for engineered MAGE A3-directed T cells. Sci. Transl. Med. 5, 197ra103 (2013).
12. Linette, G. P. et al. Cardiovascular toxicity and titin cross-reactivity of affinity-enhanced T cells in myeloma and melanoma. Blood 122, 863-871 (2013).
13. Raman, M. C. C. et al. Direct molecular mimicry enables off-target cardiovascular toxicity by an enhanced affinity TCR designed for cancer immunotherapy. Sci. Rep. 6, 18851 (2016).
14. Bijen, H. M. et al. Preclinical Strategies to Identify Off-Target Toxicity of High-Affinity TCRs. Mol. Ther. (2018). doi: 10.1016/J.YMTHE.2018.02.017
15. Shao, W. et al. The SysteMHC Atlas project. Nucleic Acids Res. 46, D1237-D1247 (2018).
16. Zacharias, M. & Springer, S. Conformational Flexibility of the MHC Class I α1-α2 Domain in Peptide Bound and Free States: A Molecular Dynamics Simulation Study. Biophys. J. 87, 2203-2214 (2004).
17. Hein, Z. et al. Peptide-independent stabilization of MHC class I molecules breaches cellular quality control. J. Cell Sci. 127, 2885-97 (2014).
18. Saini, S. K. et al. Not all empty MHC class I molecules are molten globules: Tryptophan fluorescence reveals a two-step mechanism of thermal denaturation. Mol. Immunol. 54, 386-396 (2013).
19. Saini, S. K. et al. Dipeptides promote folding and peptide binding of MHC class I molecules. Proc. Natl. Acad. Sci. 110, 15383-15388 (2013).
20. Boulter, J. M. et al. Stable, soluble T-cell receptor molecules for crystallization and therapeutics. Protein Eng. 16, 707-711 (2003).
21. Chen, J.-L. et al. Structural and kinetic basis for heightened immunogenicity of T cell vaccines. J. Exp. Med. 201, 1243-55 (2005).
22. Aggen, D. H. et al. Identification and engineering of human variable regions that allow expression of stable single-chain T cell receptors. Protein Eng. Des. Sel. 24, 361-372 (2011).
23. Zhu, Z. & Carter, P. Identification of heavy chain residues in a humanized anti-CD3 antibody important for efficient antigen binding and T cell activation. J. Immunol. 155, 1903-10 (1995).
24. Burrows, S. R., Rodda, S. J., Suhrbier, A., Geysen, H. M. & Moss, D. J. The specificity of recognition of a cytotoxic T lymphocyte epitope. Eur. J. Immunol. 22, 191-195 (1992).
25. Rodenko, B. et al. Generation of peptide-MHC class I complexes through UV-mediated ligand exchange. Nat. Protoc. 1, 1120-1132 (2006).
26. Andreatta, M. & Nielsen, M. Gapped sequence alignment using artificial neural networks: application to the MHC class I system. Bioinformatics 32, 511-517 (2016).
27. Thomsen, M. C. F. & Nielsen, M. Seq2Logo: a method for construction and visualization of amino acid binding motifs and sequence profiles including sequence weighting, pseudo counts and two-sided representation of amino acid enrichment and depletion. Nucleic Acids Res. 40, W281-7 (2012).
28. Weinschenk, T. et al. Integrated functional genomics approach for the design of patient-individual antitumor vaccines. Clin. Cancer Res. 62, 5818-5827 (2002).
29. Fritsche, J. et al. Translating Immunopeptidomics to Immunotherapy-Decision-Making for Patient and Personalized Target Selection. Proteomics 18, 1700284 (2018).
30. Elliott, T., Willis, A., Cerundolo, V. & Townsend, A. Processing of major histocompatibility class I-restricted antigens in the endoplasmic reticulum. J. Exp. Med. 181, 1481-1491 (1995).
31. Bossi, G. et al. Examining the presentation of tumor-associated antigens on peptide-pulsed T2 cells. Oncoimmunology 2, e26840 (2013).
32. Birnbaum, M. E. et al. Deconstructing the peptide-MHC specificity of t cell recognition. Cell 157, 1073-1087 (2014).
33. Gee, M. H. et al. Antigen Identification for Orphan T Cell Receptors Expressed on Tumor-Infiltrating Lymphocytes. Cell 172, 549-563.e16 (2018).
34 Cole, D. K. et al. Dual molecular mechanisms govern escape at immunodominant HLA A2-restricted HIV epitope. Front. Immunol. 8, 1503 (2017).
35. Kabsch, W. XDS. Acta Crystallogr. D Biol. Crystalogr. 66, 125-132 (2010).
36. Evans, P. Scaling and assessment of data quality. Acta Crystallogr. Sect. D Biol. Crystallogr. 62, 72-82 (2006).
37. Vagin, A. et al. Molecular replacement with MOLREP. Acta Crystollogr. Sect. D Biol. Crystallogr. 66, 22-25 (2010).
38. Kovalevskiy, O. et al. Overview of refinement procedures within REFMAC 5: Utilizing data from different sources. Acta Crystallogr. Sect. D Struct. Biol. 74, 215-227 (2018).
39. Emsley, P. et al. Features and development of Coot. Acta Crystallogr. Sect. D Biol. Crystallogr. 66, 486-501 (2010).
40. Chen, V. B. et al. MolProbity: All-atom structure validation for macromolecular crystallography. Acta Crystallogr. Sect. D Biol. Crystallogr. 66, 12-21 (2010).
41. S. K. Saini, T. Tamhane, R. Anjanappa, A. Saikia, S. Ramskov, M. Donia, I. M. Svane, S. N. Jakobsen, M. Garcia-Alai, M. Zacharias, R. Meijers, S. Springer, S. R. Hadrup, Empty peptide-receptive MHC class I molecules for efficient detection of antigen-specific T cells. Sci. Immunol. 4, 37 eaau9039, 07-19 (2019).

1. A method for screening for a TCR-binding peptide ligand/MHC molecule complex (pMHC), comprising:
a) providing a stabilized human HLA-A*0201 molecule capable of binding to a peptide ligand,
wherein said HLA-A molecule comprises an artificially introduced covalent bridge between a first amino acid within the β1 unit at the amino acid position 22 in the alpha 1 domain and a second amino acid within the α1 unit at the amino acid position 71 in the alpha 1 domain of the HLA-A molecule based on IGMT numbering excluding the first 24 amino acids,
wherein the first and the second amino acids are each modified to a cysteine forming a first disulfide bridge,
wherein the first amino acid at position 22 is modified from phenylalanine (F) to cysteine (C) and the second amino acid at position 71 is modified from serine(S) to cysteine (C), and wherein the first disulfide bridge stabilizes the HLA-A molecule in the absence of the peptide ligand,
b) contacting said suitably stabilized MHC molecule with a multitude of peptide ligands thereof, to form peptide ligand/MHC (pMHC) molecule complexes, and
c) screening said pMHC molecule complexes for TCR-binding.

2. The method of claim 1, wherein the HLA-A molecule further comprises a third amino acid modified in the α1 unit of the alpha 1 domain at the amino acid position 51 and a fourth amino acid modified within the alpha 2 domain at the amino acid position 175 based on IGMT numbering excluding the first 24 amino acids, wherein the third and the fourth amino acids are each modified to a cysteine forming a second disulfide bridge.

3. The method of claim 1, wherein the polypeptide comprises SEQ ID NO: 324.

4. The method of claim 2, wherein the polypeptide comprises SEQ ID NO: 325.

5. The method of claim 1, wherein said loaded HLA/peptide molecules are stable for more than about 1 day at about 4° C.

6. The method of claim 1, wherein said loaded HLA/peptide molecules are stable for more than 1 week at about 4° C.

7. The method of claim 1, wherein the sensitivity level for affinity screening of a TCR for binding to pMHC complexes is higher than about K_{d} 1.0×10⁻⁹.

8. The method of claim 1, wherein the sensitivity level for affinity screening of a TCR for binding to pMHC complexes is higher than about K_{d} 1.0×10⁻⁶.

9. The method of claim 1, wherein the sensitivity level for affinity screening of a TCR for binding to pMHC complexes is higher than about K_{d} 1.0×10⁻³.

10. The method of claim 1, wherein either the TCR or the MHC molecule is suitably immobilized on a solid surface selected from the group consisting of a chip, biosensor, glass slide and bead.

11. The method of claim 1, wherein said method is performed as a high-throughput screening format.

12. A method for detecting or generating a specific amino acid binding motif for a TCR, comprising
(a) performing the method according to claim 1 comprising a preselected TCR, and
(b) determining and comparing the amino acid sequences of those peptide ligands in said peptide ligand/MHC molecule complexes for which a TCR binding was detected, thereby identifying the specific amino acid binding motif for said preselected TCR.

13. A specific amino acid binding motif as determined in claim 12.

14. The method according to claim 12, wherein said peptide ligand/MHC molecule complexes are used in parallel assay reactions having different concentrations, optionally, wherein said method steps are repeated comprising a pool of peptides consisting of modified amino acid binding motifs for said preselected TCR as identified.

15. A method for detecting or determining cross-reactivity of a TCR, comprising
a) performing the method according to claim 12, and
b) determining and comparing the amino acid sequences of those peptide ligands in said peptide ligand/MHC molecule complexes for which a TCR binding was detected, thereby identifying cross-reactivity of said TCR.

16. A method for detecting or determining cross-reactivity of a TCR, comprising
a) performing the method according to claim 1 comprising a preselected TCR, and
b) determining and comparing the amino acid sequences of those peptide ligands in said peptide ligand/MHC molecule complexes for which a TCR binding was detected, thereby identifying cross-reactivity of said TCR.

17. The method according to claim 1, further comprising measuring T cell activation comprising a TCR and a TCR-binding peptide ligand/MHC molecule complex that binds said TCR.

18. A nucleic acid molecule encoding a polypeptide comprising a stabilized human HLA-A*0201 molecule capable of binding to a peptide ligand,
wherein said HLA-A molecule comprises an artificially introduced covalent bridge between a first amino acid within the β1 unit at the amino acid position 22 in the alpha 1 domain and a second amino acid within the α1 unit at the amino acid position 71 in the alpha 1 domain of the HLA-A molecule based on IGMT numbering excluding the first 24 amino acids,
wherein the first and the second amino acids are each modified to a cysteine forming a first disulfide bridge,
wherein the first amino acid at position 22 is modified from phenylalanine (F) to cysteine (C) and the second amino acid at position 71 is modified from serine(S) to cysteine (C), and
wherein the first disulfide bridge stabilizes the HLA-A molecule in the absence of the peptide ligand.

19. The nucleic acid molecule of claim 18, wherein the HLA-A molecule further comprises a third amino acid modified in the α1 unit of the alpha 1 domain at the amino acid position 51 and a fourth amino acid modified within the alpha 2 domain at the amino acid position 175 based on IGMT numbering excluding the first 24 amino acids, wherein the third and the fourth amino acids are each modified to a cysteine forming a second disulfide bridge.

20. The nucleic acid molecule of claim 18, wherein the polypeptide comprises SEQ ID NO: 324.

21. The nucleic acid molecule of claim 19, wherein the polypeptide comprises SEQ ID NO: 325.


SEQUENCE LISTING (331 sequences)

SEQ ID NO: 1 - protein, 9 aa, Homo sapiens
ALYNVLAKV

SEQ ID NO: 2 - protein, 6 aa, Homo sapiens
APPVAG

SEQ ID NO: 3 - protein, 9 aa, Homo sapiens
SLLMWITQC

SEQ ID NO: 4 - protein, 9 aa, Homo sapiens
SLLMWITQV

SEQ ID NO: 5 - protein, 9 aa, Homo sapiens
SLYNTVATL

SEQ ID NO: 6 - protein, 365 aa, Homo sapiens
MAVMAPRTLLLLLSGALALTQTWAGSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 7 - protein, 362 aa, Homo sapiens
MLVMAPRTVLLLLSAALALTETWAGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 8 - protein, 366 aa, Homo sapiens
MRVMAPRTLILLLSGALALTETWACSHSMKYFFTSVSRPGRGEPRFISVGYVDDTQFVRFDSDAASPRGEPRAPWVEQEGPEYWDRETQKYKRQAQTDRVSLRNLRGYYNQSEAGSHTLQWMCGCDLGPDGRLLRGYDQYAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVMVPSGEEQRYTCHVQHEGLPEPLTLRWEPSSQPTIPIVGIVAGLAVLAVLAVLGAVVAVVMCRRKSSGGKGGSCSQAASSNSAQGSDESLIACKA

SEQ ID NO: 9 - protein, 358 aa, Homo sapiens [sequence omitted]

SEQ ID NO: 10 - protein, 346 aa, Homo sapiens
MAPRSLLLLLSGALALTDTWAGSHSLRYFSTAVSRPGRGEPRYIAVEYVDDTQFLRFDSDAAIPRMEPREPWVEQEGPQYWEWTTGYAKANAQTDRVALRNLLRRYNQSEAGSHTLQGMNGCDMGPDGRLLRGYHQHAYDGKDYISLNEDLRSWTAADTVAQITQRFYEAEEYAEEFRTYLEGECLELLRRYLENGKETLQRADPPKAHVAHHPISDHEATLRCWALGFYPAEITLTWQRDGEEQTQDTELVETRPAGDGTFQKWAAVVVPSGEEQRYTCHVQHEGLPQPLILRWEQSPQPTIPIVGIVAGLVVLGAVVTGAVVAAVMWRKKSSDRNRGSYSQAAV

SEQ ID NO: 11 - protein, 338 aa, Homo sapiens
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 12 - protein, 363 aa, Homo sapiens
  VARIANT 363: note = any amino acid
MVLMAPRTLLLLLSGALALTQTWARSHSMRYFYTTMSRPGRGEPRFISVGYVDDTQFVRFDSDAASQRMEPRAPWMEREGPEYWDRNTQICKAQAQTERENLRIALRYYNQSEGGSHTMQVMYGCDVGPDGRFLRGYEQHAYDSKDYIALNEDLRSWTAADMAAQITKRKWEAARQAEQLRAYLEGEFVEWLRRYLENGKETLQRADPPKTHMTHHPISDHEATLRCWALGFYPAEITLTWQRDGEDQTHTRSSWRPGLQGMEPSRSGRLWWCLLERSRDTPAMCSMRVCQSPSPDGSHLPSPPSPSWASLLAWFYLLWSLELWSLLCGGRRAQIEKEGATLRLQAATVPRALMCLSRRESVX

SEQ ID NO: 13 - protein, 356 aa, Homo sapiens
MGSWRPEPSSCCSRGPWPWPRPGRAPTPGISAPPFPGRAAGSPASLPWATWTTRSSCGSTVTPVGRRGRGGWSRRGRSIGTYRHWAPRPRHRLTETCGPCSATTTRARRGITSSRECLAATWGPTGVSSAGMSSMPTTARITSPTRTCAPGPPRIPRLRLPSASMRRPMWLSKGEPTWRAPAWSGSADTWRTGRRRCSARTPPKTHVTHPPLTGITRSWVLGFYPAEITLTWQRDGEDQTQDMELVETRPTGDGTFQKWAVVVVPSGEEQRYTCHVQHKGLPKPLILRWEPSPQPTIPIVGIIAGLVLLGAVVTGAVVTAVMWRKKSSDRKGGSYSQAASSQSAQGSDVSLTACKV

SEQ ID NO: 14 - protein, 357 aa, Homo sapiens
MGSWRPEPSSCCSWGPWPPRPGRVPTPGISAPPCPGRVAGSPGTSQWATWTTRSSCGSTATRRLRGCSRSRRGWSRRDRSIGTGAHGTSGPRTDQETCPCRAATTTRARPGLTPSRCMAATWGWKGASSAGMNSTPTMARITPGTRTCAPGPRRTWRLRSPSASGRQKNLQSRSGPTWRARAWRGSQTPGEREGDAAAHGPLPQTHMIHHSVSDYKATLRCWALGFYPVEITLAWQQDGEDQTRDMELLETRPAGDGTFQKWAAVVVPSGEEQRYPCHVQHEGLPKPLTLRWEQSSQPTIPIVGIVAGLVLLGAVVTGAVVSAVMCRKKNSDRVSYSEAASSDHAQGSDVSLTACKV

SEQ ID NO: 15 - protein, 357 aa, Homo sapiens
  VARIANT 357: note = any amino acid
MGVMAPRTLLLLLLGALALTETWAGSHSLRYFSTAVSQPGRGEPRFIAVGYVDDTEFVRFDSDSVSPRMERRAPWVEQEGLEYWDQETRNAKGHAQIYRVNLRTLLRYYNQSEAGSHTIQRKHGCDVGPTGASSAGMNSSPTMARITSPTRTCTPGPPRTQRLRSPSTSGKRTNTQSRSGPTGQVHGVAPQTPGEREGDAAARGSPKGTCDPAPHLPGHPEVLGPGPLPCGDHTDLAAGWGGPDPGHGACGDQACRGRNLPEVGGCSGAFRRGAEIHVPCAAGAARAPHPEMGAVFSAHHPHRGHRCWPVSPWSCGHWSCGCCCDVEEEKLRNKEELCSGCLQQLCSVLCISYLSLX

SEQ ID NO: 16 - protein, 9 aa, synthetic construct
  REGION 1..9: note = Positional scanning library sequence
GLYNTVATL

SEQ ID NO: 17 - protein, 9 aa, synthetic construct
  REGION 1..9: note = Positional scanning library sequence
PLYNTVATL

SEQ ID NO: 18 - protein, 9 aa, synthetic construct
  REGION 1..9: note = Positional scanning library sequence
ALYNTVATL

SEQ ID NO: 19 - protein, 9 aa, synthetic construct
  REGION 1..9: note = Positional scanning library sequence
VLYNTVATL

SEQ ID NO: 20 - protein, 9 aa, synthetic construct
  REGION 1..9: note = Positional scanning library sequence
LLYNTVATL

SEQ ID NO: 21 - protein, 9 aa, synthetic construct
  REGION 1..9: note = Positional scanning library sequence
ILYNTVATL

SEQ ID NO: 22 - protein, 9 aa, synthetic construct
  REGION 1..9: note = Positional scanning library sequence
MLYNTVATL

SEQ ID NO: 23 - protein, 9 aa, synthetic construct
  REGION 1..9: note = Positional scanning library sequence
FLYNTVATL

SEQ ID NO: 24 - protein, 9 aa, synthetic construct
  REGION 1..9: note = Positional scanning library sequence
YLYNTVATL

SEQ ID NO: 25 - protein, 9 aa, synthetic construct
  REGION 1..9: note = Positional scanning library sequence
WLYNTVATL

SEQ ID NO: 26 - protein, 9 aa, synthetic construct
  REGION 1..9: note = Positional scanning library sequence
HLYNTVATL

SEQ ID NO: 27 - protein, 9 aa, synthetic construct
  REGION 1..9: note = Positional scanning library sequence
KLYNTVATL

SEQ ID NO: 28 - protein, 9 aa, synthetic construct
  REGION 1..9: note = Positional scanning library sequence
RLYNTVATL

SEQ ID NO: 29 - protein, 9 aa, synthetic construct
  REGION 1..9: note = Positional scanning library sequence
QLYNTVATL

SEQ ID NO: 30 - protein, 9 aa, synthetic construct
  REGION 1..9: note = Positional scanning library sequence
NLYNTVATL

SEQ ID NO: 31 - protein, 9 aa, synthetic construct
  REGION 1..9: note = Positional scanning library sequence
ELYNTVATL

SEQ ID NO: 32 - protein, 9 aa, synthetic construct
  REGION 1..9: note = Positional scanning library sequence
DLYNTVATL

SEQ ID NO: 33 - protein, 9 aa, synthetic construct
  REGION 1..9: note = Positional scanning library sequence
TLYNTVATL

SEQ ID NO: 34 - protein, 9 aa, synthetic construct
  REGION 1..9: note = Positional scanning library sequence
SGYNTVATL

SEQ ID NO: 35 - protein, 9 aa, synthetic construct
  REGION 1..9: note = Positional scanning library sequence
SPYNTVATL

SEQ ID NO: 36 - protein, 9 aa, synthetic construct
  REGION 1..9: note = Positional scanning library sequence
SAYNTVATL

SEQ ID NO: 37 - protein, 9 aa, synthetic construct
  REGION 1..9: note = Positional scanning library sequence
SVYNTVATL

SEQ ID NO: 38 - protein, 9 aa, synthetic construct
  REGION 1..9: note = Positional scanning library sequence
SIYNTVATL

SEQ ID NO: 39 - protein, 9 aa, synthetic construct
  REGION 1..9: note = Positional scanning library sequence
SMYNTVATL

SEQ ID NO: 40 - protein, 9 aa, synthetic construct
  REGION 1..9: note = Positional scanning library sequence
SFYNTVATL

SEQ ID NO: 41 - protein, 9 aa, synthetic construct
  REGION 1..9: note = Positional scanning library sequence
SYYNTVATL

SEQ ID NO: 42 - protein, 9 aa, synthetic construct
  REGION 1..9: note = Positional scanning library sequence
SWYNTVATL

SEQ ID NO: 43 - protein, 9 aa, synthetic construct
  REGION 1..9: note = Positional scanning library sequence
SHYNTVATL

SEQ ID NO: 44 - protein, 9 aa, synthetic construct
  REGION 1..9: note = Positional scanning library sequence
SKYNTVATL

SEQ ID NO: 45 - protein, 9 aa, synthetic construct
  REGION 1..9: note = Positional scanning library sequence
SRYNTVATL

SEQ ID NO: 46 - protein, 9 aa, synthetic construct
  REGION 1..9: note = Positional scanning library sequence
SQYNTVATL

SEQ ID NO: 47 - protein, 9 aa, synthetic construct
  REGION 1..9: note = Positional scanning library sequence
SNYNTVATL

SEQ ID NO: 48 - protein, 9 aa, synthetic construct
  REGION 1..9: note = Positional scanning library sequence
SEYNTVATL

SEQ ID NO: 49 - protein, 9 aa, synthetic construct
  REGION 1..9: note = Positional scanning library sequence
SDYNTVATL

SEQ ID NO: 50 - protein, 9 aa, synthetic construct
  REGION 1..9: note = Positional scanning library sequence
SSYNTVATL

SEQ ID NO: 51 - protein, 9 aa, synthetic construct
  REGION 1..9: note = Positional scanning library sequence
STYNTVATL

SEQ ID NO: 52 - protein, 9 aa, synthetic construct
  REGION 1..9: note = Positional scanning library sequence
SLGNTVATL

SEQ ID NO: 53 - protein, 9 aa, synthetic construct
  REGION 1..9: note = Positional scanning library sequence
SLPNTVATL

SEQ ID NO: 54 - protein, 9 aa, synthetic construct
  REGION 1..9: note = Positional scanning library sequence
SLANTVATL

SEQ ID NO: 55 - protein, 9 aa, synthetic construct
  REGION 1..9: note = Positional scanning library sequence
SLVNTVATL

SEQ ID NO: 56 - protein, 9 aa, synthetic construct
  REGION 1..9: note = Positional scanning library sequence
SLLNTVATL

SEQ ID NO: 57 - protein, 9 aa, synthetic construct
  REGION 1..9: note = Positional scanning library sequence
SLINTVATL

SEQ ID NO: 58 - protein, 9 aa, synthetic construct
  REGION 1..9: note = Positional scanning library sequence
SLMNTVATL

SEQ ID NO: 59 - protein, 9 aa, synthetic construct
  REGION 1..9: note = Positional scanning library sequence
SLFNTVATL

SEQ ID NO: 60 - protein, 9 aa, synthetic construct
  REGION 1..9: note = Positional scanning library sequence
SLWNTVATL

SEQ ID NO: 61 - protein, 9 aa, synthetic construct
  REGION 1..9: note = Positional scanning library sequence
SLHNTVATL

SEQ ID NO: 62 - protein, 9 aa, synthetic construct
  REGION 1..9: note = Positional scanning library sequence
SLKNTVATL

SEQ ID NO: 63 - protein, 9 aa, synthetic construct
  REGION 1..9: note = Positional scanning library sequence
SLRNTVATL

SEQ ID NO: 64 - protein, 9 aa, synthetic construct
  REGION 1..9: note = Positional scanning library sequence
SLQNTVATL

SEQ ID NO: 65 - protein, 9 aa, synthetic construct
  REGION 1..9: note = Positional scanning library sequence
SLNNTVATL

SEQ ID NO: 66 - protein, 9 aa, synthetic construct
  REGION 1..9: note = Positional scanning library sequence
SLENTVATL

SEQ ID NO: 67 - protein, 9 aa, synthetic construct
  REGION 1..9: note = Positional scanning library sequence
SLDNTVATL

SEQ ID NO: 68 - protein, 9 aa, synthetic construct
  REGION 1..9: note = Positional scanning library sequence
SLSNTVATL

SEQ ID NO: 69 - protein, 9 aa, synthetic construct
  REGION 1..9: note = Positional scanning library sequence
SLTNTVATL

SEQ ID NO: 70 - protein, 9 aa, synthetic construct
  REGION 1..9: note = Positional scanning library sequence
SLYGTVATL

SEQ ID NO: 71 - protein, 9 aa, synthetic construct
  REGION 1..9: note = Positional scanning library sequence
SLYPTVATL

SEQ ID NO: 72 - protein, 9 aa, synthetic construct
  REGION 1..9: note = Positional scanning library sequence
SLYATVATL

SEQ ID NO: 73 - protein, 9 aa, synthetic construct
  REGION 1..9: note = Positional scanning library sequence
SLYVTVATL

SEQ ID NO: 74 - protein, 9 aa, synthetic construct
  REGION 1..9: note = Positional scanning library sequence
SLYLTVATL

SEQ ID NO: 75 - protein, 9 aa, synthetic construct
  REGION 1..9: note = Positional scanning library sequence
SLYITVATL

SEQ ID NO: 76 - protein, 9 aa, synthetic construct
  REGION 1..9: note = Positional scanning library sequence
SLYMTVATL

SEQ ID NO: 77 - protein, 9 aa, synthetic construct
  REGION 1..9: note = Positional scanning library sequence
SLYFTVATL

SEQ ID NO: 78 - protein, 9 aa, synthetic construct
  REGION 1..9: note = Positional scanning library sequence
SLYYTVATL

SEQ ID NO: 79 - protein, 9 aa, synthetic construct
  REGION 1..9: note = Positional scanning library sequence
SLYWTVATL

SEQ ID NO: 80 - protein, 9 aa, synthetic construct
  REGION 1..9: note = Positional scanning library sequence
SLYHTVATL

SEQ ID NO: 81 - protein, 9 aa, synthetic construct
  REGION 1..9: note = Positional scanning library sequence
SLYKTVATL

SEQ ID NO: 82 - protein, 9 aa, synthetic construct
  REGION 1..9: note = Positional scanning library sequence
SLYRTVATL

SEQ ID NO: 83 - protein, 9 aa, synthetic construct
  REGION 1..9: note = Positional scanning library sequence
SLYQTVATL

SEQ ID NO: 84 - protein, 9 aa, synthetic construct
  REGION 1..9: note = Positional scanning library sequence
SLYETVATL

SEQ ID NO: 85 - protein, 9 aa, synthetic construct
  REGION 1..9: note = Positional scanning library sequence
SLYDTVATL

SEQ ID NO: 86 - protein, 9 aa, synthetic construct
  REGION 1..9: note = Positional scanning library sequence
SLYSTVATL

SEQ ID NO: 87 - protein, 9 aa, synthetic construct
  REGION 1..9: note = Positional scanning library sequence
SLYTTVATL

SEQ ID NO: 88 - protein, 9 aa, synthetic construct
  REGION 1..9: note = Positional scanning library sequence
SLYNGVATL

SEQ ID NO: 89 - protein, 9 aa, synthetic construct
  REGION 1..9: note = Positional scanning library sequence
SLYNPVATL

SEQ ID NO: 90 - protein, 9 aa, synthetic construct
  REGION 1..9: note = Positional scanning library sequence
SLYNAVATL

SEQ ID NO: 91 - protein, 9 aa, synthetic construct
  REGION 1..9: note = Positional scanning library sequence
SLYNVVATL

SEQ ID NO: 92 - protein, 9 aa, synthetic construct
  REGION 1..9: note = Positional scanning library sequence
SLYNLVATL

SEQ ID NO: 93 - protein, 9 aa, synthetic construct
  REGION 1..9: note = Positional scanning library sequence
SLYNIVATL

SEQ ID NO: 94 - protein, 9 aa, synthetic construct
  REGION 1..9: note = Positional scanning library sequence
SLYNMVATL

SEQ ID NO: 95 - protein, 9 aa, synthetic construct
  REGION 1..9: note = Positional scanning library sequence
SLYNFVATL

SEQ ID NO: 96 - protein, 9 aa, synthetic construct
  REGION 1..9: note = Positional scanning library sequence
SLYNYVATL

SEQ ID NO: 97 - protein, 9 aa, synthetic construct
  REGION 1..9: note = Positional scanning library sequence
SLYNWVATL

SEQ ID NO: 98 - protein, 9 aa, synthetic construct
  REGION 1..9: note = Positional scanning library sequence
SLYNHVATL

SEQ ID NO: 99 - protein, 9 aa, synthetic construct
  REGION 1..9: note = Positional scanning library sequence
SLYNKVATL

SEQ ID NO: 100 - protein, 9 aa, synthetic construct
  REGION 1..9: note = Positional scanning library sequence
SLYNRVATL

SEQ ID NO: 101 - protein, 9 aa, synthetic construct
  REGION 1..9: note = Positional scanning library sequence
SLYNQVATL

SEQ ID NO: 102 - protein, 9 aa, synthetic construct
  REGION 1..9: note = Positional scanning library sequence
SLYNNVATL

SEQ ID NO: 103 - protein, 9 aa, synthetic construct
  REGION 1..9: note = Positional scanning library sequence
SLYNEVATL

SEQ ID NO: 104 - protein, 9 aa, synthetic construct
  REGION 1..9: note = Positional scanning library sequence
SLYNDVATL

SEQ ID NO: 105 - protein, 9 aa, synthetic construct
  REGION 1..9: note = Positional scanning library sequence
SLYNSVATL

SEQ ID NO: 106 - protein, 9 aa, synthetic construct
  REGION 1..9: note = Positional scanning library sequence
SLYNTGATL

SEQ ID NO: 107 - protein, 9 aa, synthetic construct
  REGION 1..9: note = Positional scanning library sequence
SLYNTPATL

SEQ ID NO: 108 - protein, 9 aa, synthetic construct
  REGION 1..9: note = Positional scanning library sequence
SLYNTAATL

SEQ ID NO: 109 - protein, 9 aa, synthetic construct
  REGION 1..9: note = Positional scanning library sequence
SLYNTLATL

SEQ ID NO: 110 - protein, 9 aa, synthetic construct
  REGION 1..9: note = Positional scanning library sequence
SLYNTIATL

SEQ ID NO: 111 - protein, 9 aa, synthetic construct
  REGION 1..9: note = Positional scanning library sequence
SLYNTMATL

SEQ ID NO: 112 - protein, 9 aa, synthetic construct
  REGION 1..9: note = Positional scanning library sequence
SLYNTFATL

SEQ ID NO: 113 - protein, 9 aa, synthetic construct
  REGION 1..9: note = Positional scanning library sequence
SLYNTYATL

SEQ ID NO: 114 - protein, 9 aa, synthetic construct
  REGION 1..9: note = Positional scanning library sequence
SLYNTWATL

SEQ ID NO: 115 - protein, 9 aa, synthetic construct
  REGION 1..9: note = Positional scanning library sequence
SLYNTHATL

SEQ ID NO: 116 - protein, 9 aa, synthetic construct
  REGION 1..9: note = Positional scanning library sequence
SLYNTKATL

SEQ ID NO: 117 - protein, 9 aa, synthetic construct
  REGION 1..9: note = Positional scanning library sequence
SLYNTRATL

SEQ ID NO: 118 - protein, 9 aa, synthetic construct
  REGION 1..9: note = Positional scanning library sequence
SLYNTQATL

SEQ ID NO: 119 - protein, 9 aa, synthetic construct
  REGION 1..9: note = Positional scanning library sequence
SLYNTNATL

SEQ ID NO: 120 - protein, 9 aa, synthetic construct
  REGION 1..9: note = Positional scanning library sequence
SLYNTEATL

SEQ ID NO: 121 - protein, 9 aa, synthetic construct
  REGION 1..9: note = Positional scanning library sequence
SLYNTDATL

SEQ ID NO: 122 - protein, 9 aa, synthetic construct
  REGION 1..9: note = Positional scanning library sequence
SLYNTSATL

SEQ ID NO: 123 - protein, 9 aa, synthetic construct
  REGION 1..9: note = Positional scanning library sequence
SLYNTTATL

SEQ ID NO: 124 - protein, 9 aa, synthetic construct
  REGION 1..9: note = Positional scanning library sequence
SLYNTVGTL

SEQ ID NO: 125 - protein, 9 aa, synthetic construct
  REGION 1..9: note = Positional scanning library sequence
SLYNTVPTL

SEQ ID NO: 126 - protein, 9 aa, synthetic construct
  REGION 1..9: note = Positional scanning library sequence
SLYNTVVTL

SEQ ID NO: 127 - protein, 9 aa, synthetic construct
  REGION 1..9: note = Positional scanning library sequence
SLYNTVLTL

SEQ ID NO: 128 - protein, 9 aa, synthetic construct
  REGION 1..9: note = Positional scanning library sequence
SLYNTVITL

SEQ ID NO: 129 - protein, 9 aa, synthetic construct
  REGION 1..9: note = Positional scanning library sequence
SLYNTVMTL

SEQ ID NO: 130 - protein, 9 aa, synthetic construct
  REGION 1..9: note = Positional scanning library sequence
SLYNTVFTL

SEQ ID NO: 131 - protein, 9 aa, synthetic construct
  REGION 1..9: note = Positional scanning library sequence
SLYNTVYTL

SEQ ID NO: 132 - protein, 9 aa, synthetic construct
  REGION 1..9: note = Positional scanning library sequence
SLYNTVWTL

SEQ ID NO: 133 - protein, 9 aa, synthetic construct
  REGION 1..9: note = Positional scanning library sequence
SLYNTVHTL

SEQ ID NO: 134 - protein, 9 aa, synthetic construct
  REGION 1..9: note = Positional scanning library sequence
SLYNTVKTL

SEQ ID NO: 135 - protein, 9 aa, synthetic construct
  REGION 1..9: note = Positional scanning library sequence
SLYNTVRTL

SEQ ID NO: 136 - protein, 9 aa, synthetic construct
  REGION 1..9: note = Positional scanning library sequence
SLYNTVQTL

SEQ ID NO: 137 - protein, 9 aa, synthetic construct
  REGION 1..9: note = Positional scanning library sequence
SLYNTVNTL

SEQ ID NO: 138 - protein, 9 aa, synthetic construct
  REGION 1..9: note = Positional scanning library sequence
SLYNTVETL

SEQ ID NO: 139 - protein, 9 aa, synthetic construct
  REGION 1..9: note = Positional scanning library sequence
SLYNTVDTL

SEQ ID NO: 140 - protein, 9 aa, synthetic construct
  REGION 1..9: note = Positional scanning library sequence
SLYNTVSTL

SEQ ID NO: 141 - protein, 9 aa, synthetic construct
  REGION 1..9: note = Positional scanning library sequence
SLYNTVTTL

SEQ ID NO: 142 - protein, 9 aa, synthetic construct
  REGION 1..9: note = Positional scanning library sequence
SLYNTVAGL

SEQ ID NO: 143 - protein, 9 aa, synthetic construct
  REGION 1..9: note = Positional scanning library sequence
SLYNTVAPL

SEQ ID NO: 144 - protein, 9 aa, synthetic construct
  REGION 1..9: note = Positional scanning library sequence
SLYNTVAAL

SEQ ID NO: 145 - protein, 9 aa, synthetic construct
  REGION 1..9: note = Positional scanning library sequence
SLYNTVAVL

SEQ ID NO: 146 - protein, 9 aa, synthetic construct
  REGION 1..9: note = Positional scanning library sequence
SLYNTVALL

SEQ ID NO: 147 - protein, 9 aa, synthetic construct
  REGION 1..9: note = Positional scanning library sequence
SLYNTVAIL

SEQ ID NO: 148 - protein, 9 aa, synthetic construct
  REGION 1..9: note = Positional scanning library sequence
SLYNTVAML

SEQ ID NO: 149 - protein, 9 aa, synthetic construct
  REGION 1..9: note = Positional scanning library sequence
SLYNTVAFL

SEQ ID NO: 150 - protein, 9 aa, synthetic construct
  REGION 1..9: note = Positional scanning library sequence
SLYNTVAYL

SEQ ID NO: 151 - protein, 9 aa, synthetic construct
  REGION 1..9: note = Positional scanning library sequence
SLYNTVAWL

SEQ ID NO: 152 - protein, 9 aa, synthetic construct
  REGION 1..9: note = Positional scanning library sequence
SLYNTVAHL

SEQ ID NO: 153 - protein, 9 aa, synthetic construct
  REGION 1..9: note = Positional scanning library sequence
SLYNTVAKL

SEQ ID NO: 154 - protein, 9 aa, synthetic construct
  REGION 1..9: note = Positional scanning library sequence
SLYNTVARL

SEQ ID NO: 155 - protein, 9 aa, synthetic construct
  REGION 1..9: note = Positional scanning library sequence
SLYNTVAQL

SEQ ID NO: 156 - protein, 9 aa, synthetic construct
  REGION 1..9: note = Positional scanning library sequence
SLYNTVANL

SEQ ID NO: 157 - protein, 9 aa, synthetic construct
  REGION 1..9: note = Positional scanning library sequence
SLYNTVAEL

SEQ ID NO: 158 - protein, 9 aa, synthetic construct
  REGION 1..9: note = Positional scanning library sequence
SLYNTVADL

SEQ ID NO: 159 - protein, 9 aa, synthetic construct
  REGION 1..9: note = Positional scanning library sequence
SLYNTVASL

SEQ ID NO: 160 - protein, 9 aa, synthetic construct
  REGION 1..9: note = Positional scanning library sequence
SLYNTVATG

SEQ ID NO: 161 - protein, 9 aa, synthetic construct
  REGION 1..9: note = Positional scanning library sequence
SLYNTVATP

SEQ ID NO: 162 - protein, 9 aa, synthetic construct
  REGION 1..9: note = Positional scanning library sequence
SLYNTVATA

SEQ ID NO: 163 - protein, 9 aa, synthetic construct
  REGION 1..9: note = Positional scanning library sequence
SLYNTVATV

SEQ ID NO: 164 - protein, 9 aa, synthetic construct
  REGION 1..9: note = Positional scanning library sequence
SLYNTVATI

SEQ ID NO: 165 - protein, 9 aa, synthetic construct
  REGION 1..9: note = Positional scanning library sequence
SLYNTVATM

SEQ ID NO: 166 - protein, 9 aa, synthetic construct
  REGION 1..9: note = Positional scanning library sequence
SLYNTVATF

SEQ ID NO: 167 - protein, 9 aa, synthetic construct
  REGION 1..9: note = Positional scanning library sequence
SLYNTVATY

SEQ ID NO: 168 - protein, 9 aa, synthetic construct
  REGION 1..9: note = Positional scanning library sequence
SLYNTVATW

SEQ ID NO: 169 - protein, 9 aa, synthetic construct
  REGION 1..9: note = Positional scanning library sequence
SLYNTVATH

SEQ ID NO: 170 - protein, 9 aa, synthetic construct
  REGION 1..9: note = Positional scanning library sequence
SLYNTVATK

SEQ ID NO: 171 - protein, 9 aa, synthetic construct
  REGION 1..9: note = Positional scanning library sequence
SLYNTVATR

SEQ ID NO: 172 - protein, 9 aa, synthetic construct
  REGION 1..9: note = Positional scanning library sequence
SLYNTVATQ

SEQ ID NO: 173 - protein, 9 aa, synthetic construct
  REGION 1..9: note = Positional scanning library sequence
SLYNTVATN

SEQ ID NO: 174 - protein, 9 aa, synthetic construct
  REGION 1..9: note = Positional scanning library sequence
SLYNTVATE

SEQ ID NO: 175 - protein, 9 aa, synthetic construct
  REGION 1..9: note = Positional scanning library sequence
SLYNTVATD

SEQ ID NO: 176 - protein, 9 aa, synthetic construct
  REGION 1..9: note = Positional scanning library sequence
SLYNTVATS

SEQ ID NO: 177 - protein, 9 aa, synthetic construct
  REGION 1..9: note = Positional scanning library sequence
SLYNTVATT

SEQ ID NO: 178 - protein, 9 aa, Homo sapiens
RVYNTVPLV

SEQ ID NO: 179 - protein, 9 aa, Homo sapiens
RMYNLVSRI

SEQ ID NO: 180 - protein, 9 aa, Homo sapiens
SLYNMVPSI

SEQ ID NO: 181 - protein, 9 aa, Homo sapiens
TVYNMVPSI

SEQ ID NO: 182 - protein, 9 aa, Homo sapiens
ALYNVIAMA

SEQ ID NO: 183 - protein, 9 aa, Homo sapiens
AIYNLLPDI

SEQ ID NO: 184 - protein, 9 aa, Homo sapiens
STYNLVSTS

SEQ ID NO: 185 - protein, 9 aa, Homo sapiens
SVYNMVPSI

SEQ ID NO: 186 - protein, 9 aa, Homo sapiens
RTYNVLAIL

SEQ ID NO: 187 - protein, 9 aa, Homo sapiens
SVYNLVSIA

SEQ ID NO: 188 - protein, 9 aa, Homo sapiens
RAYNLIGTV

SEQ ID NO: 189 - protein, 9 aa, Homo sapiens
ALFNLIPVG

SEQ ID NO: 190 - protein, 9 aa, Homo sapiens
RIYNVIGTL

SEQ ID NO: 191 - protein, 9 aa, Homo sapiens
RIYNVVGTI

SEQ ID NO: 192 - protein, 9 aa, Homo sapiens
TLFNLVPNS

SEQ ID NO: 193 - protein, 9 aa, Homo sapiens
SLFNVISIL

SEQ ID NO: 194 - protein, 9 aa, Homo sapiens
STFNLVAIS

SEQ ID NO: 195 - protein, 9 aa, Homo sapiens
TLFNLIPVG

SEQ ID NO: 196 - protein, 9 aa, Homo sapiens
TIFNLIPNS

SEQ ID NO: 197 - protein, 9 aa, Homo sapiens
ALYNVLAKV

SEQ ID NO: 198 - protein, 9 aa, Homo sapiens
AVFNLLPHT

SEQ ID NO: 199 - protein, 9 aa, Homo sapiens
RMYNLLGHM

SEQ ID NO: 200 - protein, 9 aa, Homo sapiens
STWNTPPNM

SEQ ID NO: 201 - protein, 9 aa, Homo sapiens
NIYNLIAII

SEQ ID NO: 202 - protein, 9 aa, Homo sapiens
RIYNLPPEL

SEQ ID NO: 203 - protein, 9 aa, Homo sapiens
TTFNLPSAA

SEQ ID NO: 204 - protein, 9 aa, Homo sapiens
MFFNVIAIV

SEQ ID NO: 205 - protein, 9 aa, Homo sapiens
SLWNTVSGI

SEQ ID NO: 206 - protein, 9 aa, Homo sapiens
MLWNLLALR

SEQ ID NO: 207 - protein, 9 aa, Homo sapiens
VFWNLLPTV

SEQ ID NO: 208 - protein, 9 aa, Homo sapiens
STFNTTSNG

SEQ ID NO: 209 - protein, 9 aa, Homo sapiens
GFFNLLSHV

SEQ ID NO: 210 - protein, 9 aa, Homo sapiens
LLYNVPAVA

SEQ ID NO: 211 - protein, 9 aa, Homo sapiens
ALFNTISQG

SEQ ID NO: 212 - protein, 9 aa, Homo sapiens
TTFNTLAGS

SEQ ID NO: 213 - protein, 9 aa, Homo sapiens
SLWNLLGNA

SEQ ID NO: 214 - protein, 9 aa, Homo sapiens
SLYNLLNLT

SEQ ID NO: 215 - protein, 9 aa, Homo sapiens
GVWNLLSIV

SEQ ID NO: 216 - protein, 9 aa, Homo sapiens
ALFNVVNSI

SEQ ID NO: 217 - protein, 9 aa, Homo sapiens
VIYNLLGLA

SEQ ID NO: 218 - protein, 9 aa, Homo sapiens
SIFNIVAIA

SEQ ID NO: 219 - protein, 9 aa, Homo sapiens
TVYNTVSEG

SEQ ID NO: 220 - protein, 9 aa, Homo sapiens
DLWNTLSSL

SEQ ID NO: 221 - protein, 9 aa, Homo sapiens
IFFNLLAVL

SEQ ID NO: 222 - protein, 9 aa, Homo sapiens
DLFNLLPDV

SEQ ID NO: 223 - protein, 9 aa, Homo sapiens
LSWNVVPNA

SEQ ID NO: 224 - protein, 9 aa, Homo sapiens
MLWNLLALH

SEQ ID NO: 225 - protein, 9 aa, Homo sapiens
TIFNTVNTS

SEQ ID NO: 226 - protein, 9 aa, Homo sapiens
KTFNLIPAV

SEQ ID NO: 227 - protein, 9 aa, Homo sapiens
NLFNVTPLI

SEQ ID NO: 228 - protein, 9 aa, Homo sapiens
SYWNIISTV

SEQ ID NO: 229 - protein, 9 aa, Homo sapiens
GVFNLIAVL

SEQ ID NO: 230 - protein, 9 aa, Homo sapiens
RLFNITSSA

SEQ ID NO: 231 - protein, 9 aa, Homo sapiens
NLWNLVAVI

SEQ ID NO: 232 - protein, 9 aa, Homo sapiens
RIFNLIGMM

SEQ ID NO: 233 - protein, 9 aa, Homo sapiens
RLFNVVSRG

SEQ ID NO: 234 - protein, 9 aa, Homo sapiens
LVFNVIPTL

SEQ ID NO: 235 - protein, 9 aa, Homo sapiens
TTWNILSSA

SEQ ID NO: 236 - protein, 9 aa, Homo sapiens
KLFNVLSTL

SEQ ID NO: 237 - protein, 9 aa, Homo sapiens
RVYNLTAKS

SEQ ID NO: 238 - protein, 9 aa, Homo sapiens
LTFNTISLS

SEQ ID NO: 239 - protein, 9 aa, Homo sapiens
AQFNLLSST

SEQ ID NO: 240 - protein, 9 aa, Homo sapiens
VVYNVLSEL

SEQ ID NO: 241 - protein, 9 aa, Homo sapiens
KVYNTPSTS

SEQ ID NO: 242 - protein, 9 aa, Homo sapiens
GIFNIIPST

SEQ ID NO: 243 - protein, 9 aa, Homo sapiens
NIYNTLSGL

SEQ ID NO: 244 - protein, 9 aa, Homo sapiens
RLFNLTSTF

SEQ ID NO: 245 - protein, 9 aa, Homo sapiens
TVWNTLSSL

SEQ ID NO: 246 - protein, 9 aa, Homo sapiens
RLFNMLSAV

SEQ ID NO: 247 - protein, 9 aa, Homo sapiens
SIWNVTAIA

SEQ ID NO: 248 - protein, 9 aa, Homo sapiens
ALFNLMSGI

SEQ ID NO: 249 - protein, 9 aa, Homo sapiens
IVYNLLSAM

SEQ ID NO: 250 - protein, 9 aa, Homo sapiens
ISFNMLPSI

SEQ ID NO: 251 - protein, 9 aa, Homo sapiens
NTYNILPGS

SEQ ID NO: 252 - protein, 9 aa, Homo sapiens
RLWNMVNVT

SEQ ID NO: 253 - protein, 9 aa, Homo sapiens
SAFNITSLI

SEQ ID NO: 254 - protein, 9 aa, Homo sapiens
NIFNLPNIV

SEQ ID NO: 255 - protein, 9 aa, Homo sapiens
GVYNLPGAS

SEQ ID NO: 256 - protein, 9 aa, Homo sapiens
GTYNVISLV

SEQ ID NO: 257 - protein, 9 aa, Homo sapiens
SIFNTLSDI

SEQ ID NO: 258 - protein, 9 aa, Homo sapiens
TIFNILSGI

SEQ ID NO: 259 - protein, 9 aa, Homo sapiens
LLFNLISSS

SEQ ID NO: 260 - protein, 9 aa, Homo sapiens
RTFNLTAGS

SEQ ID NO: 261 - protein, 9 aa, Homo sapiens
TVFNILPGG

SEQ ID NO: 262 - protein, 9 aa, Homo sapiens
GLFNIPPAS

SEQ ID NO: 263 - protein, 9 aa, Homo sapiens
RMFNIISDS

SEQ ID NO: 264 - protein, 9 aa, Homo sapiens
TTFNIVGTT

SEQ ID NO: 265 - protein, 9 aa, Homo sapiens
ALFNLMSGV

SEQ ID NO: 266 - protein, 9 aa, Homo sapiens
SVFNITAIA

SEQ ID NO: 267 - protein, 9 aa, Homo sapiens
KIYNTPSAS

SEQ ID NO: 268 - protein, 9 aa, Homo sapiens
LLYNLLGSS

SEQ ID NO: 269 - protein, 9 aa, Homo sapiens
SLYNMMGEA

SEQ ID NO: 270 - protein, 9 aa, Homo sapiens
SLWNLMGNA

SEQ ID NO: 271 - protein, 9 aa, Homo sapiens
GLYNIVGNA

SEQ ID NO: 272 - protein, 9 aa, Homo sapiens
LTWNLTPKA

SEQ ID NO: 273 - protein, 9 aa, Homo sapiens
LIFNVTGLA

SEQ ID NO: 274 - protein, 9 aa, Homo sapiens
SIFNITGIA

SEQ ID NO: 275 - protein, 9 aa, Homo sapiens
LTFNLVSDA

SEQ ID NO: 276 - protein, 9 aa, Homo sapiens
MQWNILAQA

SEQ ID NO: 277 - protein, 9 aa, Homo sapiens
LSWNLVPEA

SEQ ID NO: 278 - protein, 9 aa, Homo sapiens
DLWNTLSEA

SEQ ID NO: 279 - protein, 9 aa, Homo sapiens
GLFNIPPAF

SEQ ID NO: 280 - protein, 9 aa, Homo sapiens
LIWNILASF

SEQ ID NO: 281 - protein, 9 aa, Homo sapiens
LLFNMLPGG

SEQ ID NO: 282 - protein, 9 aa, Homo sapiens
LVYNIMSSG

SEQ ID NO: 283 - protein, 9 aa, Homo sapiens
IIYNVPGTG

SEQ ID NO: 284 - protein, 9 aa, Homo sapiens
VIYNVTSDG

SEQ ID NO: 285 - protein, 9 aa, Homo sapiens
GTFNLPSDG

SEQ ID NO: 286 - protein, 9 aa, Homo sapiens
KLWNTLNLI

SEQ ID NO: 287 - protein, 9 aa, Homo sapiens
LMWNIISII

SEQ ID NO: 288 - protein, 9 aa, Homo sapiens
GLFNTTSNI

SEQ ID NO: 289 - protein, 9 aa, Homo sapiens
LIFNTLSLI

SEQ ID NO: 290 - protein, 9 aa, Homo sapiens
SVFNLMNAI

SEQ ID NO: 291 - protein, 9 aa, Homo sapiens
LTFNILGQI

SEQ ID NO: 292 - protein, 9 aa, Homo sapiens
GLFNMVSSL

SEQ ID NO: 293 - protein, 9 aa, Homo sapiens
KIFNIINSL

SEQ ID NO: 294 - protein, 9 aa, Homo sapiens
AVWNVLGNL

SEQ ID NO: 295 - protein, 9 aa, Homo sapiens
KVFNIVSDL

SEQ ID NO: 296 - protein, 9 aa, Homo sapiens
DLWNVVSHL

SEQ ID NO: 297 - protein, 9 aa, Homo sapiens
LQFNTVSKL

SEQ ID NO: 298 - protein, 9 aa, Homo sapiens
MSFNTVSEL

SEQ ID NO: 299 - protein, 9 aa, Homo sapiens
ASWNIVNLL

SEQ ID NO: 300 - protein, 9 aa, Homo sapiens
ISFNIISAL

SEQ ID NO: 301 - protein, 9 aa, Homo sapiens
AFFNILNEL

SEQ ID NO: 302 - protein, 9 aa, Homo sapiens
LVFNLLPIM

SEQ ID NO: 303 - protein, 9 aa, Homo sapiens
KIFNTVPDM

SEQ ID NO: 304 - protein, 9 aa, Homo sapiens
MLFNLIGLS

SEQ ID NO: 305 - protein, 9 aa, Homo sapiens
LLFNLPPGS

SEQ ID NO: 306 - protein, 9 aa, Homo sapiens
MTFNLIGES

SEQ ID NO: 307 - protein, 9 aa, Homo sapiens
KVYNIPGIS

SEQ ID NO: 308 - protein, 9 aa, Homo sapiens
GIYNIPGDS

SEQ ID NO: 309 - protein, 9 aa, Homo sapiens
GLYNLMNIT

SEQ ID NO: 310 - protein, 9 aa, Homo sapiens
LTWNMINTT

SEQ ID NO: 311 - protein, 9 aa, Homo sapiens
IVFNVLSDT

SEQ ID NO: 312 - protein, 9 aa, Homo sapiens
IVFNVVSDT

SEQ ID NO: 313 - protein, 9 aa, Homo sapiens
LIFNITASV

SEQ ID NO: 314 - protein, 9 aa, Homo sapiens
IVFNLTNNV

SEQ ID NO: 315 - protein, 9 aa, Homo sapiens
KSFNVLSSV

SEQ ID NO: 316 - protein, 9 aa, Homo sapiens
LAFNILGMV

SEQ ID NO: 317 - protein, 9 aa, Homo sapiens
VSWNITGTV

SEQ ID NO: 318 - protein, 9 aa, synthetic construct
  REGION 1..9: note = Peptide ligand sequence
SLFNTIAVL

SEQ ID NO: 319 - protein, 8 aa, synthetic construct
  REGION 1..8: note = Peptide ligand sequence
SLFNTVAV

SEQ ID NO: 320 - protein, 9 aa, synthetic construct
  REGION 1..9: note = Peptide ligand sequence
SLFNTIATL

SEQ ID NO: 321 - protein, 9 aa, synthetic construct
  REGION 1..9: note = Peptide ligand sequence
SLYNTIAVL

SEQ ID NO: 322 - protein, 275 aa, Homo sapiens [sequence omitted]

SEQ ID NO: 323 - protein, 275 aa, synthetic construct
  REGION 1..275: note = Y84C/A139C HLA-A*02:01 variant sequence
GSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWE

SEQ ID NO: 324 - protein, 275 aa, synthetic construct
  REGION 1..275: note = F22C/S71C HLA-A*02:01 variant
GSHSMRYFFTSVSRPGRGEPRCIAVGYVDDTQFVRFDSDAASQRMEPRAPWIEQEGPEYWDGETRKVKAHCQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWE

SEQ ID NO: 325 - protein, 275 aa, synthetic construct
  REGION 1..275: note = F22C/S71C W51C/G175C HLA-A*02:01 variant
GSHSMRYFFTSVSRPGRGEPRCIAVGYVDDTQFVRFDSDAASQRMEPRAPCIEQEGPEYWDGETRKVKAHCQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENCKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWE

SEQ ID NO: 326 - protein, 6 aa, synthetic construct
  REGION 1..6: note = CDR2beta
YYEEEE

SEQ ID NO: 327 - protein, 6 aa, synthetic construct
  REGION 1..6: note = CDR2beta
YVRGEE

SEQ ID NO: 328 - protein, 12 aa, synthetic construct
  REGION 1..12: note = CDR3alpha
CAVRTNSGYALN

SEQ ID NO: 329 - protein, 12 aa, synthetic construct
  REGION 1..12: note = CDR3alpha
CAVRGAHDYALN

SEQ ID NO: 330 - protein, 17 aa, synthetic construct
  REGION 1..17: note = Linker
GSADDAKKDAAKKDGKS

SEQ ID NO: 331 - protein, 339 aa, Mus musculus
GPHSLRYFVTAVSRPGLGEPRYMEVGYVDDTEFVRFDSDAENPRYEPRARWMEQEGPEYWERETQKAKGNEQSFRVDLRTLLGYYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDSPKAHVTHHSRPEDKVTLRCWALGFYPADITLTWQLNGEELIQDMELVETRPAGDGTFQKWASVVVPLGKEQYYTCHVYHQGLPEPLTLRWEPPPSTVSNMATVAVLVVLGAAIVTGAVVAFVMKMRRRNTGGKGGDYALAPGSQTSDLSLPDCKA